SEQUENCE LISTING (138 sequences)

SEQ ID NO: 1 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 2 - protein, 1053 aa, Staphylococcus aureus
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1053 aa, Staphylococcus aureus
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - RNA, 105 nt, Artificial Sequence
  chemically synthesized
  misc_feature (1)..(21): n is a, c, g, or u
nnnnnnnnnnnnnnnnnnnnnguuuuaguacucuguaaugaaaauuacagaaucuacuaaaacaaggcaaaaugccguguuuaucucgucaacuuguuggcgaga

SEQ ID NO: 6 - RNA, 107 nt, Artificial Sequence
  chemically synthesized
  misc_feature (1)..(21): n is a, c, g, or u
nnnnnnnnnnnnnnnnnnnnnguuuaaguacucugugcuggaaacagcacagaaucuacuuaaacaaggcaaaaugccguguuuaucucgucaacuuguuggcgaga

SEQ ID NO: 7 - RNA, 100 nt, Artificial Sequence
  chemically synthesized
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 8 - RNA, 96 nt, Artificial Sequence
  chemically synthesized
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 9 - RNA, 114 nt, Artificial Sequence
  chemically synthesized
  modified_base (1)..(30): n is a, c, g, u, or other and this region may encompass 17-30 nucleotides;
  misc_feature (1)..(30): n is a, c, g, or u
  modified_base (107)..(114): This region may encompass 1-8 urasil;
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 10 - RNA, 104 nt, Artificial Sequence
  chemically synthesized
  misc_feature (1)..(20): n is a, c, g, or u
nnnnnnnnnnnnnnnnnnnnguuuuaguacucuguaaugaaaauuacagaaucuacuaaaacaaggcaaaaugccguguuuaucucgucaacuuguuggcgaga

SEQ ID NO: 11 - RNA, 106 nt, Artificial Sequence
  chemically synthesized
  misc_feature (1)..(20): n is a, c, g, or u
nnnnnnnnnnnnnnnnnnnnguuuaaguacucugugcuggaaacagcacagaaucuacuuaaacaaggcaaaaugccguguuuaucucgucaacuuguuggcgaga

SEQ ID NO: 12 - RNA, 111 nt, Artificial Sequence
  chemically synthesized
  misc_feature (1)..(20): n is a, c, g, or u
nnnnnnnnnnnnnnnnnnnnguuuuaguacucuguaaugaaaauuacagaaucuacuaaaacaaggcaaaaugccguguuuaucucgucaacuuguuggcgagauuuuuuu

SEQ ID NO: 13 - RNA, 113 nt, Artificial Sequence
  chemically synthesized
  misc_feature (1)..(20): n is a, c, g, or u
nnnnnnnnnnnnnnnnnnnnguuuaaguacucugugcuggaaacagcacagaaucuacuuaaacaaggcaaaaugccguguuuaucucgucaacuuguuggcgagauuuuuuu

SEQ ID NO: 14 - RNA, 122 nt, Artificial Sequence
  chemically synthesized
  modified_base (1)..(30): n is a, c, g, u, or other and this region may encompass 17-30 nucleotides;
  misc_feature (1)..(30): n is a, c, g, or u
  modified_base (107)..(114): This region may encompass 1-8 urasil;
nnnnnnnnnnnnnnnnnnnnnnnnnnnnnnguuuuaguacucuguaaugaaaauuacagaaucuacuaaaacaaggcaaaaugccguguuuaucucgucaacuuguuggcgagauuuuuuuu

SEQ ID NO: 15 - RNA, 124 nt, Artificial Sequence
  chemically synthesized
  modified_base (1)..(30): n is a, c, g, u, or other and this region may encompass 17-30 nucleotides;
  misc_feature (1)..(30): n is a, c, g, or u
  modified_base (107)..(114): This region may encompass 1-8 urasil;
nnnnnnnnnnnnnnnnnnnnnnnnnnnnnnguuuaaguacucugugcuggaaacagcacagaaucuacuuaaacaaggcaaaaugccguguuuaucucgucaacuuguuggcgagauuuuuuuu

SEQ ID NO: 16 - DNA, 27 bp, Artificial Sequence
  chemically synthesized
gtgtattgcttgtactactcactgaat

SEQ ID NO: 17 - DNA, 27 bp, Artificial Sequence
  chemically synthesized
gtgttattacttgctactgcagagagt

SEQ ID NO: 18 - DNA, 27 bp, Artificial Sequence
  chemically synthesized
cttagaggtcttctacatacaatgagt

SEQ ID NO: 19 - DNA, 27 bp, Artificial Sequence
  chemically synthesized
ctattctgagtacagagcatacagagt

SEQ ID NO: 20 - DNA, 27 bp, Artificial Sequence
  chemically synthesized
actatgattaaatgcttgatattgagt

SEQ ID NO: 21 - DNA, 27 bp, Artificial Sequence
  chemically synthesized
cttaaaggcttcatataagggtggaat

SEQ ID NO: 22 - RNA, 105 nt, Artificial Sequence
  chemically synthesized
guguauugcuuguacuacucaguuuuaguacucuguaaugaaaauuacagaaucuacuaaaacaaggcaaaaugccguguuuaucucgucaacuuguuggcgaga

SEQ ID NO: 23 - RNA, 105 nt, Artificial Sequence
  chemically synthesized
guguuauuacuugcuacugcaguuuuaguacucuguaaugaaaauuacagaaucuacuaaaacaaggcaaaaugccguguuuaucucgucaacuuguuggcgaga

SEQ ID NO: 24 - RNA, 107 nt, Artificial Sequence
  chemically synthesized
cuuagaggucuucuacauacaguuuaaguacucugugcuggaaacagcacagaaucuacuuaaacaaggcaaaaugccguguuuaucucgucaacuuguuggcgaga

SEQ ID NO: 25 - RNA, 107 nt, Artificial Sequence
  chemically synthesized
cuauucugaguacagagcauaguuuaaguacucugugcuggaaacagcacagaaucuacuuaaacaaggcaaaaugccguguuuaucucgucaacuuguuggcgaga

SEQ ID NO: 26 - RNA, 107 nt, Artificial Sequence
  chemically synthesized
acuaugauuaaaugcuugauaguuuaaguacucugugcuggaaacagcacagaaucuacuuaaacaaggcaaaaugccguguuuaucucgucaacuuguuggcgaga

SEQ ID NO: 27 - RNA, 107 nt, Artificial Sequence
  chemically synthesized
cuuaaaggcuucauauaagggguuuaaguacucugugcuggaaacagcacagaaucuacuuaaacaaggcaaaaugccguguuuaucucgucaacuuguuggcgaga

SEQ ID NO: 28 - DNA, 23 bp, Artificial Sequence
  chemically synthesized
  misc_feature (1)..(21): n is a, c, g, or t
nnnnnnnnnnnnnnnnnnnnnrg

SEQ ID NO: 29 - DNA, 24 bp, Artificial Sequence
  chemically synthesized
  misc_feature (1)..(22): n is a, c, g, or t
nnnnnnnnnnnnnnnnnnnnnnrg

SEQ ID NO: 30 - DNA, 26 bp, Artificial Sequence
  chemically synthesized
  misc_feature (1)..(22): n is a, c, g, or t
nnnnnnnnnnnnnnnnnnnnnngrrt

SEQ ID NO: 31 - DNA, 27 bp, Artificial Sequence
  chemically synthesized
  misc_feature (1)..(23): n is a, c, g, or t
nnnnnnnnnnnnnnnnnnnnnnngrrt

SEQ ID NO: 32 - DNA, 26 bp, Artificial Sequence
  chemically synthesized
  misc_feature (1)..(22): n is a, c, g, or t
  misc_feature (26)..(26): n is a, c, g, or t
nnnnnnnnnnnnnnnnnnnnnngrrn

SEQ ID NO: 33 - DNA, 27 bp, Artificial Sequence
  chemically synthesized
  misc_feature (1)..(23): n is a, c, g, or t
  misc_feature (27)..(27): n is a, c, g, or t
nnnnnnnnnnnnnnnnnnnnnnngrrn

SEQ ID NO: 34 - DNA, 21 bp, Artificial Sequence
  chemically synthesized
acaataagtcaaatttaattg

SEQ ID NO: 35 - DNA, 21 bp, Artificial Sequence
  chemically synthesized
aagatatataatgtcatgaat

SEQ ID NO: 36 - DNA, 21 bp, Artificial Sequence
  chemically synthesized
gtgtattgcttgtactactca

SEQ ID NO: 37 - DNA, 20 bp, Artificial Sequence
  chemically synthesized
tctcctcattagagaagaag

SEQ ID NO: 38 - DNA, 21 bp, Artificial Sequence
  chemically synthesized
ctcaagcttctcagggacacc

SEQ ID NO: 39 - DNA, 21 bp, Artificial Sequence
  chemically synthesized
tcttgcatcttgcacatgtcc

SEQ ID NO: 40 - DNA, 21 bp, Artificial Sequence
  chemically synthesized
cttagaggtcttctacataca

SEQ ID NO: 41 - DNA, 21 bp, Artificial Sequence
  chemically synthesized
ttctgactgtaagtacactat

SEQ ID NO: 42 - DNA, 21 bp, Artificial Sequence
  chemically synthesized
aaattggcacagacaacttag

SEQ ID NO: 43 - DNA, 21 bp, Artificial Sequence
  chemically synthesized
aaaaacaagaagtgaggcaga

SEQ ID NO: 44 - DNA, 21 bp, Artificial Sequence
  chemically synthesized
gtgttattacttgctactgca

SEQ ID NO: 45 - DNA, 21 bp, Artificial Sequence
  chemically synthesized
acacttccttgtgacgggttt

SEQ ID NO: 46 - DNA, 21 bp, Artificial Sequence
  chemically synthesized
ctattctgagtacagagcata

SEQ ID NO: 47 - DNA, 20 bp, Artificial Sequence
  chemically synthesized
caataagtcaaatttaattg

SEQ ID NO: 48 - DNA, 20 bp, Artificial Sequence
  chemically synthesized
agatatataatgtcatgaat

SEQ ID NO: 49 - DNA, 20 bp, Artificial Sequence
  chemically synthesized
tgtattgcttgtactactca

SEQ ID NO: 50 - DNA, 20 bp, Artificial Sequence
  chemically synthesized
tcaagcttctcagggacacc

SEQ ID NO: 51 - DNA, 20 bp, Artificial Sequence
  chemically synthesized
cttgcatcttgcacatgtcc

SEQ ID NO: 52 - DNA, 20 bp, Artificial Sequence
  chemically synthesized
ttagaggtcttctacataca

SEQ ID NO: 53 - DNA, 20 bp, Artificial Sequence
  chemically synthesized
tctgactgtaagtacactat

SEQ ID NO: 54 - DNA, 20 bp, Artificial Sequence
  chemically synthesized
aattggcacagacaacttag

SEQ ID NO: 55 - DNA, 20 bp, Artificial Sequence
  chemically synthesized
aaaacaagaagtgaggcaga

SEQ ID NO: 56 - DNA, 20 bp, Artificial Sequence
  chemically synthesized
tgttattacttgctactgca

SEQ ID NO: 57 - DNA, 20 bp, Artificial Sequence
  chemically synthesized
cacttccttgtgacgggttt

SEQ ID NO: 58 - DNA, 20 bp, Artificial Sequence
  chemically synthesized
tattctgagtacagagcata

SEQ ID NO: 59 - RNA, 112 nt, Artificial Sequence
  chemically synthesized
  misc_feature (1)..(21): n is a, c, g, or u
nnnnnnnnnnnnnnnnnnnnnguuuuaguacucuguaaugaaaauuacagaaucuacuaaaacaaggcaaaaugccguguuuaucucgucaacuuguuggcgagauuuuuuu

SEQ ID NO: 60 - RNA, 114 nt, Artificial Sequence
  chemically synthesized
  misc_feature (1)..(21): n is a, c, g, or u
nnnnnnnnnnnnnnnnnnnnnguuuaaguacucugugcuggaaacagcacagaaucuacuuaaacaaggcaaaaugccguguuuaucucgucaacuuguuggcgagauuuuuuu

SEQ ID NO: 61 - RNA, 107 nt, Artificial Sequence
  chemically synthesized
guguauugcuuguacuacucaguuuaaguacucugugcuggaaacagcacagaaucuacuuaaacaaggcaaaaugccguguuuaucucgucaacuuguuggcgaga

SEQ ID NO: 62 - RNA, 107 nt, Artificial Sequence
  chemically synthesized
guguuauuacuugcuacugcaguuuaaguacucugugcuggaaacagcacagaaucuacuuaaacaaggcaaaaugccguguuuaucucgucaacuuguuggcgaga

SEQ ID NO: 63 - DNA, 26 bp, Artificial Sequence
  chemically synthesized
cgtaccgcttcgacgtgtacctggat

SEQ ID NO: 64 - DNA, 26 bp, Artificial Sequence
  chemically synthesized
ggacatcggaattacctccgtgggat

SEQ ID NO: 65 - DNA, 26 bp, Artificial Sequence
  chemically synthesized
cgaaaccggcagacgaacgaacggat

SEQ ID NO: 66 - DNA, 26 bp, Artificial Sequence
  chemically synthesized
tggagcagtacggcgacgaaaagaat

SEQ ID NO: 67 - DNA, 26 bp, Artificial Sequence
  chemically synthesized
gcctttcacgcgggcttcgtaggggt

SEQ ID NO: 68 - DNA, 26 bp, Artificial Sequence
  chemically synthesized
gacaggatgaaatcgtccacaagggt

SEQ ID NO: 69 - DNA, 26 bp, Artificial Sequence
  chemically synthesized
ggggttgataccggacaattccgagt

SEQ ID NO: 70 - DNA, 26 bp, Artificial Sequence
  chemically synthesized
ttgacctcgtacaggttgcccaggat

SEQ ID NO: 71 - DNA, 26 bp, Artificial Sequence
  chemically synthesized
tcccttgtcgtccttgcgcgtggagt

SEQ ID NO: 72 - DNA, 26 bp, Artificial Sequence
  chemically synthesized
gcgttgatgaccttgatcgactggat

SEQ ID NO: 73 - RNA, 107 nt, Artificial Sequence; identical to SEQ ID NO: 61
  chemically synthesized

SEQ ID NO: 74 - RNA, 106 nt, Artificial Sequence
  chemically synthesized
ggacaucggaauuaccuccgguuuaaguacucugugcuggaaacagcacagaaucuacuuaaacaaggcaaaaugccguguuuaucucgucaacuuguuggcgaga

SEQ ID NO: 75 - RNA, 106 nt, Artificial Sequence
  chemically synthesized
uggagcaguacggcgacgaaguuuaaguacucugugcuggaaacagcacagaaucuacuuaaacaaggcaaaaugccguguuuaucucgucaacuuguuggcgaga

SEQ ID NO: 76 - RNA, 106 nt, Artificial Sequence
  chemically synthesized
gccuuucacgcgggcuucguguuuaaguacucugugcuggaaacagcacagaaucuacuuaaacaaggcaaaaugccguguuuaucucgucaacuuguuggcgaga

SEQ ID NO: 77 - RNA, 106 nt, Artificial Sequence
  chemically synthesized
gggguugauaccggacaauuguuuaaguacucugugcuggaaacagcacagaaucuacuuaaacaaggcaaaaugccguguuuaucucgucaacuuguuggcgaga

SEQ ID NO: 78 - RNA, 106 nt, Artificial Sequence
  chemically synthesized
gcguugaugaccuugaucgaguuuaaguacucugugcuggaaacagcacagaaucuacuuaaacaaggcaaaaugccguguuuaucucgucaacuuguuggcgaga

SEQ ID NO: 79 - DNA, 3159 bp, Staphylococcus aureus
ggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaaggga

SEQ ID NO: 80 - RNA, 21 nt, Artificial Sequence
  chemically synthesized
guguauugcuuguacuacuca

SEQ ID NO: 81 - RNA, 21 nt, Artificial Sequence
  chemically synthesized
guguuauuacuugcuacugca

SEQ ID NO: 82 - RNA, 21 nt, Artificial Sequence
  chemically synthesized
cuuagaggucuucuacauaca

SEQ ID NO: 83 - RNA, 21 nt, Artificial Sequence
  chemically synthesized
cuauucugaguacagagcaua

SEQ ID NO: 84 - RNA, 21 nt, Artificial Sequence
  chemically synthesized
acuaugauuaaaugcuugaua

SEQ ID NO: 85 - RNA, 21 nt, Artificial Sequence
  chemically synthesized
cuuaaaggcuucauauaaggg

SEQ ID NO: 86 - RNA, 21 nt, Artificial Sequence
  chemically synthesized
guguauugcuuguacuacuca

SEQ ID NO: 87 - RNA, 20 nt, Artificial Sequence
  chemically synthesized
ggacaucggaauuaccuccg

SEQ ID NO: 88 - RNA, 20 nt, Artificial Sequence
  chemically synthesized
uggagcaguacggcgacgaa

SEQ ID NO: 89 - RNA, 20 nt, Artificial Sequence
  chemically synthesized
gccuuucacgcgggcuucgu

SEQ ID NO: 90 - RNA, 20 nt, Artificial Sequence
  chemically synthesized
gggguugauaccggacaauu

SEQ ID NO: 91 - RNA, 20 nt, Artificial Sequence
  chemically synthesized
gcguugaugaccuugaucga

SEQ ID NO: 92 - DNA, 4674 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 93 - DNA, 4726 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctgtgttattacttgctactgcagagagtaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 94 - DNA, 4860 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtgtattgcttgtactactcactgaatcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctgtgttattacttgctactgcagagagtaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 95 - DNA, 4923 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgccaccatggccccaaagaagaagcggaaggtcggatccggaaagcggaactatatcctgggactgggtaagtgtattgcttgtactactcactgaatcaccatcgggcgcgaagggggagacctgtagtcagagcccccgggcagcacacactgacatccactcccttcctattgtttcagacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtgttattacttgctactgcagagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 96 - DNA, 4833 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtgttattacttgctactgcagagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 97 - DNA, 4822 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtatgtgtattgcttgtactactcactgaatctacatttctcttaggtaaacatctaaggcatttcgagaacacagaaaaggttttgagtttgagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccgctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctgtgttattacttgctactgcagagagtaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 98 - DNA, 4792 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagttaatatttcatagtgtattgcttgtactactcactgaatagttactttttttttcatttttagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccgctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctgtgttattacttgctactgcagagagtaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 99 - DNA, 4972 bp, Artificial Sequence
  chemicallly synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactgggtaagtgtattgcttgtactactcactgaatcaccatcgggcgcgaagggggagacctgtagtcagagcccccgggcagcacacactgacatccactcccttcctattgtttcagacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtgttattacttgctactgcagagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccgctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctgtgttattacttgctactgcagagagtaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 100 - DNA, 4833 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccgactatgattaaatgcttgatattgagtgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacagcttaaaggcttcatataagggtggaatcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 101 - DNA, 4729 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtactcattgtatgtagaagacctctaaggccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctctattctgagtacagagcatacagagtaataaaatatctttattttcattacatctgtgtgttggttttttgtgtggtaaccacgtgcggaccgaggctgcagcgtcgtcctccctaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 102 - DNA, 4729 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtactcattgtatgtagaagacctctaaggccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctactcattgtatgtagaagacctctaagaataaaatatctttattttcattacatctgtgtgttggttttttgtgtggtaaccacgtgcggaccgaggctgcagcgtcgtcctccctaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 103 - DNA, 4952 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttagaggtcttctacatacagtttaagtactctgtgctggaaacagcacagaatctacttaaacaaggcaaaatgccgtgtttatctcgtcaacttgttggcgagatttttttcaccggtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtctaaggtactagtaataaaatatctttattttcattacatctgtgtgttggttttttgtgtgagcgctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattctgagtacagagcatagtttaagtactctgtgctggaaacagcacagaatctacttaaacaaggcaaaatgccgtgtttatctcgtcaacttgttggcgagatttttttggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 104 - DNA, 130 bp, Artificial Sequence
  chemically synthesized
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 105 - DNA, 72 bp, Artificial Sequence
  chemically synthesized
ggtgtggaaagtccccaggctccccagcaggcagaagtatgcaaagcatgcatctcaattagtcagcaacca

SEQ ID NO: 106 - DNA, 304 bp, Artificial Sequence
  chemically synthesized
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 107 - DNA, 219 bp, Artificial Sequence
  chemically synthesized
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgt

SEQ ID NO: 108 - DNA, 27 bp, Artificial Sequence
  chemically synthesized
atggccccaaagaagaagcggaaggtc

SEQ ID NO: 109 - DNA, 48 bp, Artificial Sequence
  chemically synthesized
gagggcaggggaagtctgctaacatgcggggacgtggaggaaaatccc

SEQ ID NO: 110 - DNA, 402 bp, Artificial Sequence
  chemically synthesized
atggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaa

SEQ ID NO: 111 - DNA, 49 bp, Artificial Sequence
  chemically synthesized
aataaaatatctttattttcattacatctgtgtgttggttttttgtgtg

SEQ ID NO: 112 - DNA, 141 bp, Artificial Sequence
  chemically synthesized
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 113 - DNA, 133 bp, Artificial Sequence
  chemically synthesized
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 114 - DNA, 134 bp, Artificial Sequence
  chemically synthesized
gtaagtatcaaggttacaagacaggtgtattgcttgtactactcactgaatcttgtcgagacagagaagactcttgcgtttctgataggcacctattggtcttactgacatccactttgcctttctctccacag

SEQ ID NO: 115 - DNA, 134 bp, Artificial Sequence
  chemically synthesized
gtaagtatcaaggttacaagacaggtgttattacttgctactgcagagagtcttgtcgagacagagaagactcttgcgtttctgataggcacctattggtcttactgacatccactttgcctttctctccacag

SEQ ID NO: 116 - DNA, 144 bp, Artificial Sequence
  chemically synthesized
  modified_bases (26)..(61): The number of nucleotides (N) can be 20-35
  misc_feature (27)..(61): n is a, c, g, or t
gtaagtatcaaggttacaagacaggtnnnnnnnnnnnnnnnnnnnnnnnnnnnnnnnnnnncttgtcgagacagagaagactcttgcgtttctgataggcacctattggtcttactgacatccactttgcctttctctccacag

SEQ ID NO: 117 - DNA, 70 bp, Artificial Sequence
  chemically synthesized
gtatgtctacatttctcttaggtaaacatctaaggcatttcgagaacacagaaaaggttttgagtttgag

SEQ ID NO: 118 - DNA, 95 bp, Artificial Sequence
  chemically synthesized
gtatgtgtattgcttgtactactcactgaatctacatttctcttaggtaaacatctaaggcatttcgagaacacagaaaaggttttgagtttgag

SEQ ID NO: 119 - DNA, 45 bp, Artificial Sequence
  chemically synthesized
gttaatatttcataaatagttactttttttttcatttttaggaag

SEQ ID NO: 120 - DNA, 65 bp, Artificial Sequence
  chemically synthesized
gttaatatttcatagtgtattgcttgtactactcactgaatagttactttttttttcatttttag

SEQ ID NO: 121 - DNA, 105 bp, Artificial Sequence
  chemically synthesized
gtgtattgcttgtactactcagttttagtactctgtaatgaaaattacagaatctactaaaacaaggcaaaatgccgtgtttatctcgtcaacttgttggcgaga

SEQ ID NO: 122 - DNA, 105 bp, Artificial Sequence
  chemically synthesized
gtgttattacttgctactgcagttttagtactctgtaatgaaaattacagaatctactaaaacaaggcaaaatgccgtgtttatctcgtcaacttgttggcgaga

SEQ ID NO: 123 - DNA, 107 bp, Artificial Sequence
  chemically synthesized
gtgtattgcttgtactactcagtttaagtactctgtgctggaaacagcacagaatctacttaaacaaggcaaaatgccgtgtttatctcgtcaacttgttggcgaga

SEQ ID NO: 124 - DNA, 107 bp, Artificial Sequence
  chemically synthesized
gtgttattacttgctactgcagtttaagtactctgtgctggaaacagcacagaatctacttaaacaaggcaaaatgccgtgtttatctcgtcaacttgttggcgaga

SEQ ID NO: 125 - DNA, 108 bp, Artificial Sequence
  chemically synthesized
gcttagaggtcttctacatacagtttaagtactctgtgctggaaacagcacagaatctacttaaacaaggcaaaatgccgtgtttatctcgtcaacttgttggcgaga

SEQ ID NO: 126 - DNA, 108 bp, Artificial Sequence
  chemically synthesized
gctattctgagtacagagcatagtttaagtactctgtgctggaaacagcacagaatctacttaaacaaggcaaaatgccgtgtttatctcgtcaacttgttggcgaga

SEQ ID NO: 127 - DNA, 108 bp, Artificial Sequence
  chemically synthesized
gactatgattaaatgcttgatagtttaagtactctgtgctggaaacagcacagaatctacttaaacaaggcaaaatgccgtgtttatctcgtcaacttgttggcgaga

SEQ ID NO: 128 - DNA, 108 bp, Artificial Sequence
  chemically synthesized
gcttaaaggcttcatataaggggtttaagtactctgtgctggaaacagcacagaatctacttaaacaaggcaaaatgccgtgtttatctcgtcaacttgttggcgaga

SEQ ID NO: 129 - DNA, 106 bp, Artificial Sequence
  chemically synthesized
ggacatcggaattacctccggtttaagtactctgtgctggaaacagcacagaatctacttaaacaaggcaaaatgccgtgtttatctcgtcaacttgttggcgaga

SEQ ID NO: 130 - DNA, 107 bp, Artificial Sequence
  chemically synthesized
gtggagcagtacggcgacgaagtttaagtactctgtgctggaaacagcacagaatctacttaaacaaggcaaaatgccgtgtttatctcgtcaacttgttggcgaga

SEQ ID NO: 131 - DNA, 106 bp, Artificial Sequence
  chemically synthesized
gcctttcacgcgggcttcgtgtttaagtactctgtgctggaaacagcacagaatctacttaaacaaggcaaaatgccgtgtttatctcgtcaacttgttggcgaga

SEQ ID NO: 132 - DNA, 106 bp, Artificial Sequence
  chemically synthesized
ggggttgataccggacaattgtttaagtactctgtgctggaaacagcacagaatctacttaaacaaggcaaaatgccgtgtttatctcgtcaacttgttggcgaga

SEQ ID NO: 133 - DNA, 106 bp, Artificial Sequence
  chemically synthesized
gcgttgatgaccttgatcgagtttaagtactctgtgctggaaacagcacagaatctacttaaacaaggcaaaatgccgtgtttatctcgtcaacttgttggcgaga

SEQ ID NO: 134 - DNA, 106 bp, Artificial Sequence
  chemically synthesized
cgtaccgcttcgacgtgtacgtttaagtactctgtgctggaaacagcacagaatctacttaaacaaggcaaaatgccgtgtttatctcgtcaacttgttggcgaga

SEQ ID NO: 135 - DNA, 106 bp, Artificial Sequence
  chemically synthesized
cgaaaccggcagacgaacgagtttaagtactctgtgctggaaacagcacagaatctacttaaacaaggcaaaatgccgtgtttatctcgtcaacttgttggcgaga

SEQ ID NO: 136 - DNA, 106 bp, Artificial Sequence
  chemically synthesized
gacaggatgaaatcgtccacgtttaagtactctgtgctggaaacagcacagaatctacttaaacaaggcaaaatgccgtgtttatctcgtcaacttgttggcgaga

SEQ ID NO: 137 - DNA, 106 bp, Artificial Sequence
  chemically synthesized
ttgacctcgtacaggttgccgtttaagtactctgtgctggaaacagcacagaatctacttaaacaaggcaaaatgccgtgtttatctcgtcaacttgttggcgaga

SEQ ID NO: 138 - DNA, 106 bp, Artificial Sequence
  chemically synthesized
tcccttgtcgtccttgcgcggtttaagtactctgtgctggaaacagcacagaatctacttaaacaaggcaaaatgccgtgtttatctcgtcaacttgttggcgaga

(19) United States
(12) Patent Application Publication
(10) Pub. No.: US 2021/0032622 A1
(43) Pub. Date: Feb. 4, 2021

(54) SELF-INACTIVATING (SIN) CRISPR/CAS OR CRISPR/CPF1 SYSTEMS AND USES THEREOF

(71) Applicant: CRISPR THERAPEUTICS AG, Zug (CH)

(72) Inventors: Seshidhar Reddy POLICE, Cambridge, MA (US); Song CHOU, Cambridge, MA (US); Thomas James CRADICK, Cambridge, MA (US); Robert NG, Cambridge, MA (US)

(21) Appl. No.: 16/762,444

(22) PCT Filed: Nov. 9, 2018
(86) PCT No.: PCT/IB2018/001422

§ 371 Date: May 7, 2020

Related U.S. Application Data
(60) Provisional application No. 62/592,769, filed on Nov. 30, 2017.
(60) Provisional application No. 62/583,647, filed on Nov. 9, 2017.

(51) Int. Cl. C12N 15/113; C12N 15/10; C12N 15/86; C12N 9/22

(52) U.S. Cl. CPC C12N 15/113; C12N 15/102; C12N 2310/20; C12N 9/22; C12N 2750/14143; C12N 15/86

(57) ABSTRACT

The invention relates to self-inactivating/self-targeting CRISPR/Cas or CRISPR/Cpf1 systems. The invention also relates to genetically modified cells related to the same. The invention also relates to methods of controlling Cas9 expression in a cell related to the same. The invention also relates to methods of genetically modifying a cell related to the same. The invention also relates to nucleic acids for use in a self-inactivating CRISPR/Cas or CRISPR/Cpf1 system. The invention also relates to pharmaceutical compositions related to the same.

### RELATED APPLICATIONS

This application claims the benefit of U.S. Provisional Application No. 62/583,647 filed Nov. 9, 2017; and U.S. Provisional Application No. 62/592,769 filed Nov. 30, 2017, each of which is incorporated herein in their entirety by reference.

### FIELD

The present application provides self-inactivating (SIN) CRISPR/Cas or CRISPR/Cpf1 systems and uses thereof.

### INCORPORATION BY REFERENCE OF SEQUENCE LISTING

This application contains a Sequence Listing in computer readable form (filename: CRTN-005BPC sequence listing: 149976 bytes—ASCII text file; created Nov. 9, 2018), which is incorporated herein by reference in its entirety and forms part of the disclosure.

### BACKGROUND

Editing genomes using the RNA-guided DNA targeting principle of CRISPR-Cas (Clustered Regularly Interspaced Short Palindromic Repeats-CRISPR associated proteins) immunity has been exploited widely over the past few months (1-13). The significant advantage provided by the bacterial type II CRISPR-Cas system lies in the minimal requirement for programmable DNA interference: an endonuclease, Cas9, guided by a customizable dual-RNA structure (14). As initially demonstrated in the original type II system of Streptococcus pyogenes, trans-activating CRISPR RNA (tracrRNA) (15,16) binds to the invariable repeats of precursor CRISPR RNA (pre-crRNA) forming a dual-RNA (14-17) that is essential for both RNA co-maturation by RNase III in the presence of Cas9 (15-17), and invading DNA cleavage by Cas9 (14,15,17-19). As demonstrated in Streptococcus, Cas9 guided by the duplex formed between mature activating tracrRNA and targeting crRNA (14-16) introduces site-specific double-stranded DNA (dsDNA) breaks in the invading cognate DNA (14,17-19). Cas9 is a multi-domain enzyme (14,20,21) that uses an HNH nuclease domain to cleave the target strand (defined as complementary to the spacer sequence of crRNA) and a RuvC-like domain to cleave the non-target strand (14,22,23), enabling the conversion of the dsDNA cleaving Cas9 into a nickase by selective motif inactivation (2,8,14,24,25). DNA cleavage specificity is determined by two parameters: the variable, spacer-derived sequence of crRNA targeting the protospacer sequence and a short sequence, the Protospacer Adjacent Motif (PAM), located immediately downstream of the protospacer on the non-target DNA strand (14,18,23,26-28).

Recent studies have demonstrated that RNA-guided Cas9 can be employed as an efficient genome editing tool in a wide range of species, including human cells (1,2,8,11), mice (9,10), zebrafish (6), drosophila (5), worms (4), plants (12,13), yeast (3) and bacteria (7). The system is versatile, enabling multiplex genome engineering by programming Cas9 to edit several sites in a genome simultaneously by simply using multiple guide RNAs (2,7,8,10). The easy conversion of Cas9 into a nickase was shown to facilitate homology-directed repair in mammalian genomes with reduced mutagenic activity (2,8,24,25). In addition, the DNA-binding activity of a Cas9 catalytic inactive mutant has been exploited to engineer RNA-programmable transcriptional silencing and activating devices (29,30).

To date, RNA-guided Cas9 from S. pyogenes, Streptococcus thermophilus, Neisseria meningitidis and Treponema denticola have been described as tools for genome manipulation (1-13,24,25,31-34 and Esvelt et al. PMID: 24076762).

A range of nucleases have been used for gene editing applications, including, both natural and engineered, homing endonucleases, and other types of meganuclease.

In recent years, engineered nuclease enzymes designed to target specific DNA sequences have attracted considerable attention as powerful tools for the genetic manipulation of cells and whole organisms, allowing targeted gene deletion, replacement and repair, as well as the insertion of exogenous sequences (transgenes) into the genome. Two major technologies for engineering site-specific DNA nucleases have emerged, Zinc Finger Nucleases and TAL effector nucleases (TALENs), both of which are based on the construction of chimeric endonuclease enzymes in which a sequence non-specific DNA endonuclease domain is fused to an engineered DNA binding domain (PMID: 23664777). However, targeting each new genomic locus requires the design, construction and evaluation of DNA binding domains fused to endonuclease domain, making these approaches both time-consuming and costly. In addition, both technologies suffer from limited precision, which can lead to unpredictable off-target effects.

The systematic interrogation of genomes and genetic reprogramming of cells involves targeting sets of genes for expression or repression. In recent years, the most common approach for targeting arbitrary genes for regulation is to use RNA interference (RNAi). This approach has limitations. For example, RNAi can exhibit significant off-target effects and toxicity.

There is a need in the field for a technology that allows for controlling gene expression with minimal off-target effects.

### SUMMARY

Provided herein is a self-inactivating CRISPR-Cas system comprising: a first segment comprising a nucleotide sequence that encodes a site-directed polypeptide, a second segment comprising a nucleotide sequence that encodes a DNA-targeting nucleic acid; and one or more third segments comprising a nucleotide sequence that is substantially complementary to the nucleotide sequence of the DNA-targeting nucleic acid.

The site-directed polypeptide can be Cas9 or any variants thereof. The site-directed polypeptide can be Staphylococcus aureus Cas9 (SaCas9) or any variants thereof.

The DNA-targeting nucleic acid can be a guide RNA (gRNA) or single-molecule guide RNA (sgRNA).

The one or more third segments can comprise a SIN site. The one or more third segments can comprise a protospacer adjacent motif (PAM). The PAM can be NNGRRT or any variants thereof.

The first segment can comprise a nucleotide sequence that encodes a site-directed polypeptide and further comprises a start codon, a stop codon, and a poly(A) termination site. The nucleic acid that encodes the site-directed polypeptide can further comprise one or more naturally occurring or chimeric introns inserted into, upstream, and/or downstream of a Cas9 open reading frame (ORF).

The one or more third segments can be located at any one or more of: a) a 5′ end of the first segment, upstream of the start codon and/or downstream of the transcriptional start site; b) within one or more naturally occurring or chimeric inserted introns; or c) a 3′ end of the first segment between the stop codon and poly(A) termination site.

The site-directed polypeptide can be SaCas9 and the DNA-targeting nucleic acid can be a gRNA or sgRNA that targets the one or more third segments, wherein the one or more third segments is located at the 5′ end of the first segment, upstream of the start codon, and/or downstream of the transcriptional start site.

The site-directed polypeptide can be SaCas9 and the DNA-targeting nucleic acid can be a gRNA or sgRNA that targets the one or more third segments, wherein the one or more third segments is located within one or more naturally occurring or chimeric inserted introns.

The site-directed polypeptide can be SaCas9 and the DNA-targeting nucleic acid can be a gRNA or sgRNA that targets the one or more third segments, wherein the one or more third segments is located at the 3′ end of the first segment between the stop codon and poly(A) termination site.

The site-directed polypeptide can be SaCas9 and the DNA-targeting nucleic acid can be a gRNA or sgRNA that targets the one or more third segments, wherein the one or more third segments is located at the 5′ end of the first segment, upstream of the start codon, and/or downstream of the transcriptional start site; and at the 3′ end of the first segment between the stop codon and poly(A) termination site.

The site-directed polypeptide can be SaCas9 and the DNA-targeting nucleic acid can be a gRNA or sgRNA that targets the one or more third segments, wherein the one or more third segments is located at the 5′ end of the first segment, upstream of the start codon, and/or downstream of the transcriptional start site; and within one or more naturally occurring or chimeric inserted introns.

The site-directed polypeptide can be SaCas9 and the DNA-targeting nucleic acid can be a gRNA or sgRNA that targets the one or more third segments, wherein the one or more third segments is located at the 3′ end of the first segment between the stop codon and poly(A) termination site; and within one or more naturally occurring or chimeric inserted introns.

The site-directed polypeptide can be SaCas9 and the DNA-targeting nucleic acid can be a gRNA or sgRNA that targets the one or more third segments, wherein the one or more third segments is located at the 5′ end of the first segment, upstream of the start codon, and/or downstream of the transcriptional start site; the 3′ end of the first segment between the stop codon and poly(A) termination site; and within one or more naturally occurring or chimeric inserted introns.

The first segment and the third segment can be provided together in a first vector and the second segment can be provided in a second vector. The first segment, second segment, and third segment can be provided together in a first vector. The third segment can be present in the first or second vector at a location 5′ of the first segment. The third segment can be present in the first or second vector at a location 3′ of the first segment. The one or more third segments can be present in the first or second vector at the 5′ and 3′ ends of the first segment.

The third segment can be less than 100 nucleotides in length. The third segment can be less than 50 nucleotides in length. The third segment can be less than 25 nucleotides in length.

The third segment cannot be fully complementary to the nucleotide sequence of the DNA-targeting nucleic acid in at least one location. The third segment cannot be fully complementary to the nucleotide sequence of the DNA-targeting nucleic acid in at least two locations.

A nucleic acid sequence encoding a promoter can be operably linked to the first segment. The promoter can be a spatially-restricted promoter, bidirectional promoter driving gRNA in one direction and Cas9 in the opposite orientation, or an inducible promoter. The spatially-restricted promoter can be selected from the group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a retinal pigment epithelial (RPE) selective promoter.

The Cas9 can comprise a nucleotide sequence encoding a Cas9 protein as set forth in SEQ ID NO. 1. The SaCas9 can comprise a nucleotide sequence as set forth in SEQ ID NO: 79.

The Cas9 variant can comprise a D10A mutation in the amino acid sequence set forth in SEQ ID NO: 2. The Cas9 variant can comprise a N580A mutation in the amino acid sequence set forth in SEQ ID NO: 3. The Cas9 variant can comprise both a D10A mutation and a N580A mutation in the amino acid sequence set forth in SEQ ID NO: 4.

The vector can be one or more adeno-associated virus (AAV) vectors. The adeno-associated virus (AAV) vector can be of any AAV capsid type including AAV2.

Also provided herein is a genetically modified cell. The genetically modified cell can comprise any of the self-inactivating CRISPR-Cas systems disclosed herein. The genetically modified cell can be selected from the group consisting of: an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell

Also provided herein is a method of controlling Cas9 expression in a cell. The method comprises: contacting the cell with any of the self-inactivating CRISPR-Cas systems disclosed herein. The method can further comprise contacting the cell with a third vector comprising a nucleotide sequence encoding a homology-directed repair (HDR) template.

Also provided herein is a pharmaceutical composition. The pharmaceutical composition can comprise any of the self-inactivating CRISPR-Cas systems disclosed herein. The pharmaceutical composition can be sterile.

Also provided herein is a self-inactivating CRISPR-Cas system comprising: a first segment comprising a nucleotide sequence that encodes a site-directed polypeptide; and a second segment comprising a nucleotide sequence that encodes a DNA-targeting nucleic acid; wherein the nucleotide sequence of the first segment comprises a SIN site that is substantially complementary to a DNA-targeting segment (e.g., spacer) of the DNA-targeting nucleic acid.

The site-directed polypeptide can be Cas9 or any variants thereof. The site-directed polypeptide can be Staphylococcus aureus Cas9 (SaCas9) or any variants thereof. The site-directed polypeptide can be encoded by a sequence that is 90% identical to a nucleotide sequence that encodes wild-type SaCas9.

The DNA-targeting nucleic acid can be a guide RNA (gRNA) or single-molecule guide RNA (sgRNA). The gRNA or sgRNA can comprise a sequence selected from the group consisting of SEQ ID NOs: 80 to 91. The sgRNA can comprise a sequence selected from the group consisting of SEQ ID NOs: 74 to 78.

The first segment can comprise a nucleotide sequence that encodes a site-directed polypeptide, further comprises: a start codon, a stop codon, and a poly(A) termination site. The SIN site can be located between the start codon and the stop codon. The SIN site can comprise a sequence selected from the group consisting of SEQ ID NOs: 63 to 72.

The first segment can be provided in a first vector and the second segment can be provided in a second vector. Alternatively, the first segment and second segment can be provided together in the same vector.

The DNA-targeting segment (e.g., spacer) of a DNA-targeting nucleic acid may not be fully complementary to the nucleotide sequence of the SIN site in at least one location. The DNA-targeting segment of a DNA-targeting nucleic acid may not be fully complementary to the nucleotide sequence of the SIN site in at least two locations.

A nucleic acid sequence encoding a promoter can be operably linked to the first segment. The promoter can be a spatially-restricted promoter, bidirectional promoter driving gRNA in one direction and Cas9 in the opposite orientation, or an inducible promoter. The spatially-restricted promoter can be selected from the group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a retinal pigment epithelial (RPE) selective promoter.

The first segment can comprise a nucleotide sequence encoding a Cas9 protein comprising an amino acid sequence selected from the group consisting of SEQ ID NOs: 1-4. The first segment can comprise a nucleotide sequence encoding a Cas9 protein comprising the amino acid sequence of SEQ ID NO: 1. The Cas9 variant can comprise a D10A mutation in the amino acid sequence set forth in SEQ ID NO: 2. The Cas9 variant can comprise an N580A mutation in the amino acid sequence set forth in SEQ ID NO: 3. The Cas9 variant can comprise both a D10A mutation and an N580A mutation in the amino acid sequence set forth in SEQ ID NO: 4.

The vector can be one or more adeno-associated virus (AAV) vectors. The adeno-associated virus (AAV) vector can be AAV2.

Also provided herein is a method of genetically modifying a cell. The method comprises: contacting the cell with any of the self-inactivating CRISPR-Cas systems disclosed herein.

Also provided herein is a nucleic acid for use in a self-inactivating CRISPR-Cas system. The CRISPR-Cas system can comprise a codon optimized sequence encoding a site-directed polypeptide, wherein the codon optimized sequence further comprises a SIN site. The SIN site can comprise the PAM NNGRRT, or variants thereof. The SIN site can comprise a sequence selected from the group consisting of SEQ ID NOs: 63 to 72. The codon optimized sequence can comprise SEQ ID NO: 79.

It is understood that the inventions described in this specification are not limited to the examples summarized in this Summary. Various other aspects are described and exemplified herein.

### BRIEF DESCRIPTION OF THE FIGURES

Various aspects of self-inactivating CRISPR/Cas/Cpf1 systems and uses thereof disclosed and described in this specification can be better understood by reference to the accompanying figures, in which:

FIG. 1 depicts a self-inactivating (SIN) CRISPR/Cas9 system;

FIG. 2 depicts a Cas9gRNA ribonucleoprotein (RNP) that introduces double-stranded DNA breaks at SIN sites present in a SaCas9 expression cassette;

FIG. 3 depicts a Cas9gRNA RNP that introduces double stranded DNA breaks in a target gene;

FIGS. 4A-B show schematic diagrams of various plasmid constructs encoding SaCas9 with combinations of SIN sites and constructs with or without introns (C0-C10);

FIG. 4A is a schematic diagram of various plasmid constructs expressing SaCas9 (C0-C7);

FIG. 4B is a schematic diagram of various plasmid constructs expressing SaCas9 (C8-C10). Arrows indicate the direction of the SIN site present in the construct;

FIGS. 5A-B show immunoassay SaCas9 protein expression in HEK293T cells and myogenic cells;

FIG. 5A shows immunoassay SaCas9 protein expression in HEK293T cells;

FIG. 5B shows immunoassay SaCas9 protein expression in myogenic cells;

FIG. 6 shows an in-vitro CRISPR/Cas9 DNA digestion assay;

FIGS. 7A-B show schematic diagrams of various plasmid constructs encoding guide RNAs;

FIG. 7A is a schematic diagram of plasmids G1-G3 shown as both an a and b version. G1a-G3a encode guide RNAs comprising a sequence of SEQ ID NOs: 5 or 59. G1b-G3b encode guide RNAs comprising a sequence of SEQ ID NOs: 6 or 60;

FIG. 7B is a schematic diagram of plasmids G4-G5;

FIGS. 8A-C show protein kinetics of SaCas9 expression and editing efficiency of the human dystrophin locus exon 51 mediated by the SIN CRISPR/SaCas9 system;

FIG. 8A shows protein kinetics of SaCas9 expression mediated by the SIN CRISPR/SaCas9 system, via immunoassay;

FIG. 8B shows protein kinetics of SaCas9 expression mediated by the SIN CRISPR/SaCas9 system, via quantitative protein analysis;

FIG. 8C shows editing efficiency of the human dystrophin locus exon 51 mediated by the SIN CRISPR/SaCas9 system;

FIGS. 9A-C show protein kinetics of SaCas9 expression and editing efficiency mediated by the SIN CRISPR/SaCas9 system;

FIG. 9A shows the protein kinetics of SaCas9 expression mediated by the SIN CRISPR/SaCas9 system, via immunoassay;

FIG. 9B shows protein kinetics of SaCas9 expression mediated by the SIN CRISPR/SaCas9 system, via quantitative protein analysis;

FIG. 9C shows the editing efficiency of the human dystrophin locus exon 51 mediated by the SIN CRISPR/SaCas9 system;

FIGS. 10A-B show protein kinetics of SaCas9 expression mediated by the SIN CRISPR/SaCas9 system;

FIG. 10A shows protein kinetics of SaCas9 expression mediated by the SIN CRISPR/SaCas9 system, via immunoassay;

FIG. 10B shows protein kinetics of SaCas9 expression mediated by the SIN CRISPR/SaCas9 system, via quantitative protein analysis;

FIGS. 11A-D show self-inactivation and editing efficiency in HEK293T cells mediated by a SIN CRISPR/SaCas9 system packaged in a AAV2 dual vector;

FIG. 11A shows the protein kinetics of SaCas9 expression in HEK293T cells infected with AAV2 vectors delivering C0 or SIN CRISPR/SaCas9 systems (C2, C4, or C7);

FIG. 11B shows the protein kinetics of SaCas9 expression in HEK293T cells infected with AAV2 vectors delivering C0 or SIN CRISPR/SaCas9 systems (C2, C4, or C7) together with a plasmid construct encoding dual guide RNA expression (Gib) at a lower MOI;

FIG. 11C shows the protein kinetics of SaCas9 expression in HEK293T cells infected with AAV2 vectors delivering C0 or SIN CRISPR/SaCas9 systems (C2, C4, or C7) together with a plasmid construct encoding dual guide RNA expression (Gib) at a higher MOI;

FIG. 11D shows the editing efficiency of the human dystrophin locus exon 51 in HEK293T cells infected with AAV2 vectors delivering C0 or SIN CRISPR/SaCas9 systems (C2, C4, or C7) together with a plasmid construct encoding dual guide RNA expression (Gib) at different MOIs;

FIG. 12 depicts a self-inactivating (SIN) CRISPR/Cas9 system that introduces double-stranded DNA breaks at SIN sites located within a nucleotide sequence that encodes wild-type SaCas9;

FIGS. 13A-B show a schematic diagram of plasmid C0 and the results of an in-vitro CRISPR/Cas9 DNA digestion assay involving plasmid C0 and synthetic gRNAs that target the 10 different SIN sites located within the C0 plasmid;

FIG. 13A is a schematic diagram of plasmid C0 showing the location of 10 different SIN sites (T1-T10) located within a nucleotide sequence that encodes wild-type SaCas9;

FIG. 13B shows an in-vitro CRISPR/Cas9 DNA digestion assay;

FIGS. 14A-B show protein kinetics of SaCas9 expression mediated by the SIN CRISPR/SaCas9 system that includes universal SIN gRNAs;

FIG. 14A shows protein kinetics of SaCas9 expression mediated by the SIN CRISPR/SaCas9 system that includes universal SIN gRNAs, via immunoassay;

FIG. 14B shows protein kinetics of SaCas9 expression mediated by the SIN CRISPR/SaCas9 system that includes universal SIN gRNAs, via quantitative protein analysis;

FIG. 15 shows a schematic diagram of several AAV plasmid constructs that encode universal SIN gRNAs (G12 expresses gRNA T2, G14 expresses gRNA T4, G15 expresses gRNA T5, G17 expresses gRNA T7, and G20 expresses gRNA T10); a control plasmid, G10, that expresses a gRNA that targets a site in the human dystrophin locus (sgRNA1); a plasmid, C11, that expresses SaCas9 and gRNAs that target sites in the human dystrophin locus (sgRNA3, sgRNA4); and a plasmid, C0, that expresses SaCas9;

FIGS. 16A-B show protein kinetics of SaCas9 expression mediated by the SIN CRISPR/SaCas9 system that includes universal SIN gRNAs;

FIG. 16A shows the protein kinetics of SaCas9 expression mediated by the SIN CRISPR/SaCas9 system that includes universal SIN gRNAs, via immunoassay;

FIG. 16B shows protein kinetics of SaCas9 expression mediated by the SIN CRISPR/SaCas9 system that includes universal SIN gRNAs, via quantitative protein analysis.

### BRIEF DESCRIPTION OF THE SEQUENCE LISTING

SEQ ID NO: 1 is a wild-type S. aureus Cas9 amino acid sequence;

SEQ ID NO: 2 is a S. aureus Cas9 variant amino acid sequence that comprises a

D10 mutation;

SEQ ID NO: 3 is a S. aureus Cas9 variant amino acid sequence that comprises a N580A mutation;

SEQ ID NO: 4 is a S. aureus Cas9 variant amino acid sequence that comprises a D10 and N580A mutation;

SEQ ID NO: 5 is the “a” backbone gRNA sequence for G1a-3a;

SEQ ID NO: 6 is the “b” backbone gRNA sequence for G1b-3b;

SEQ ID NOs: 7-9 show sample S. pyogenes sgRNA sequences;

SEQ ID NOs: 10-15 show sample S. aureus sgRNA sequences;

SEQ ID NO: 16 is the sequence for SIN site 1;

SEQ ID NO: 17 is the sequence for SIN site 2;

SEQ ID NO: 18 is the sequence for SIN site 3;

SEQ ID NO: 19 is the sequence for SIN site 4;

SEQ ID NO: 20 is the sequence for SIN site 5;

SEQ ID NO: 21 is the sequence for SIN site 6;

SEQ ID NO: 22 is the sequence for sgRNA 1 (backbone “a”);

SEQ ID NO: 23 is the sequence for sgRNA 2 (backbone “a”);

SEQ ID NO: 24 is the sequence for sgRNA 3;

SEQ ID NO: 25 is the sequence for sgRNA 4;

SEQ ID NO: 26 is the sequence for sgRNA 5;

SEQ ID NO: 27 is the sequence for sgRNA 6;

SEQ ID NO: 28 is a sample gRNA for a S. pyogenes Cas9 endonuclease, wherein the gRNA comprises 20 nucleotides;

SEQ ID NO: 29 is a sample gRNA for a S. pyogenes Cas9 endonuclease, wherein the gRNA comprises 21 nucleotides;

SEQ ID NO: 30 is a sample gRNA for a S. aureus Cas9 endonuclease, wherein the gRNA comprises 20 nucleotides;

SEQ ID NO: 31 is a sample gRNA for a S. aureus Cas9 endonuclease, wherein the gRNA comprises 21 nucleotides;

SEQ ID NO: 32 is a sample gRNA for a S. aureus Cas9 endonuclease, wherein the gRNA comprises 20 nucleotides;

SEQ ID NO: 33 is a sample gRNA for a S. aureus Cas9 endonuclease, wherein the gRNA comprises 21 nucleotides;

SEQ ID NOs: 34-58 are spacer sequences from exon 51 of the DMD gene;

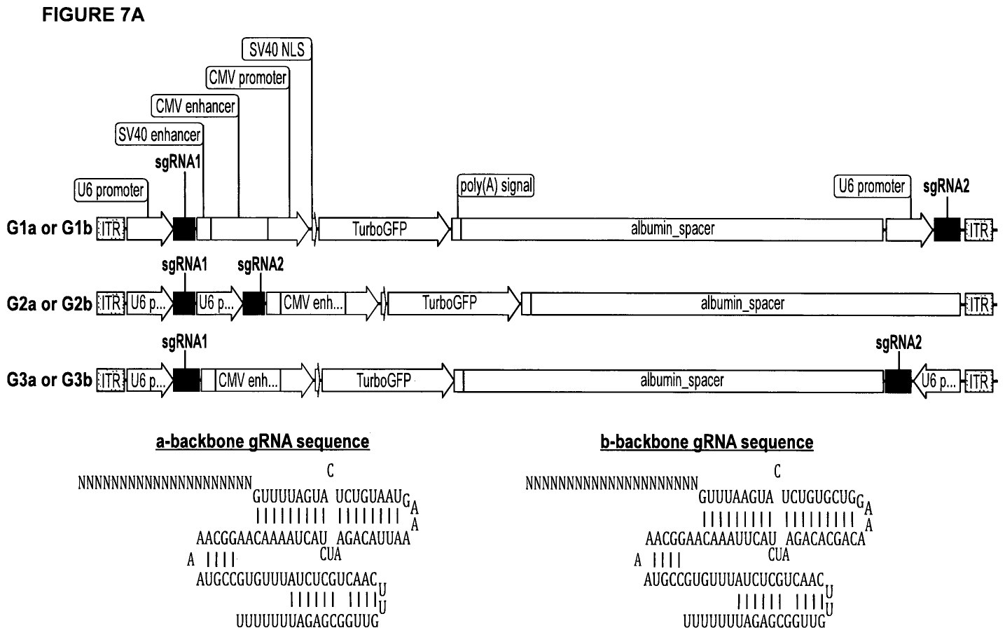

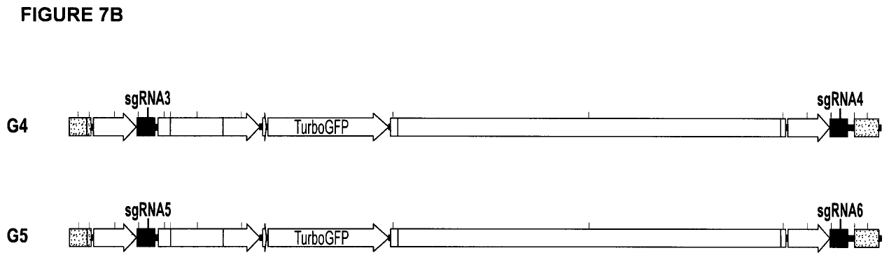

SEQ ID NO: 59 is the “a” backbone gRNA sequence for G1a-3a including a 7U tail as depicted in FIG. 7A;

SEQ ID NO: 60 is the “b” backbone gRNA sequence for G1b-3b including a 7U tail, as depicted in FIG. 7A;

SEQ ID NO: 61 is the sequence for sgRNA 1 (backbone “b”);

SEQ ID NO: 62 is the sequence for sgRNA 2 (backbone “b”);

SEQ ID NO: 63 is the sequence for SIN site T1;

SEQ ID NO: 64 is the sequence for SIN site T2;

SEQ ID NO: 65 is the sequence for SIN site T3;

SEQ ID NO: 66 is the sequence for SIN site T4;

SEQ ID NO: 67 is the sequence for SIN site T5;

SEQ ID NO: 68 is the sequence for SIN site T6;

SEQ ID NO: 69 is the sequence for SIN site T7;

SEQ ID NO: 70 is the sequence for SIN site T8;

SEQ ID NO: 71 is the sequence for SIN site T9;

SEQ ID NO: 72 is the sequence for SIN site T10;

SEQ ID NO: 73 is the sequence for a gRNA that targets a site in the human dystrophin locus;

SEQ ID NO: 74 is the sequence for a universal SIN gRNA that targets the T2 SIN site located within the SaCas9 sequence;

SEQ ID NO: 75 is the sequence for a universal SIN gRNA that targets the T4 SIN site located within the SaCas9 sequence;

SEQ ID NO: 76 is the sequence for a universal SIN gRNA that targets the T5 SIN site located within the SaCas9 sequence;

SEQ ID NO: 77 is the sequence for a universal SIN gRNA that targets the T7 SIN site located within the SaCas9 sequence;

SEQ ID NO: 78 is the sequence for a universal SIN gRNA that targets the T10 SIN site located within the SaCas9 sequence;

SEQ ID NO: 79 is the nucleotide sequence for wild-type S. aureus Cas9;

SEQ ID NO: 80 is the spacer sequence for sgRNA1;

SEQ ID NO: 81 is the spacer sequence for sgRNA2;

SEQ ID NO: 82 is the spacer sequence for sgRNA3;

SEQ ID NO: 83 is the spacer sequence for sgRNA4;

SEQ ID NO: 84 is the spacer sequence for sgRNA5;

SEQ ID NO: 85 is the spacer sequence for sgRNA6;

SEQ ID NO: 86 is the spacer sequence for the G10 sgRNA;

SEQ ID NO: 87 is the spacer sequence for the G12 sgRNA;

SEQ ID NO: 88 is the spacer sequence for the G14 sgRNA;

SEQ ID NO: 89 is the spacer sequence for the G15 sgRNA;

SEQ ID NO: 90 is the spacer sequence for the G17 sgRNA;

SEQ ID NO: 91 is the spacer sequence for the G20 sgRNA;

SEQ ID NO: 92 is the nucleotide sequence for the C0 construct;

SEQ ID NO: 93 is the nucleotide sequence for the C1 construct;

SEQ ID NO: 94 is the nucleotide sequence for the C2 construct;

SEQ ID NO: 95 is the nucleotide sequence for the C3 construct;

SEQ ID NO: 96 is the nucleotide sequence for the C4 construct;

SEQ ID NO: 97 is the nucleotide sequence for the C5 construct;

SEQ ID NO: 98 is the nucleotide sequence for the C6 construct;

SEQ ID NO: 99 is the nucleotide sequence for the C7 construct;

SEQ ID NO: 100 is the nucleotide sequence for the C8 construct;

SEQ ID NO: 101 is the nucleotide sequence for the C9 construct;

SEQ ID NO: 102 is the nucleotide sequence for the C10 construct;

SEQ ID NO: 103 is the nucleotide sequence for the C11 construct;

SEQ ID NO: 104 is the nucleotide sequence for the 5′AAV ITR component;

SEQ ID NO: 105 is the nucleotide sequence for the SV40 promoter;

SEQ ID NO: 106 is the nucleotide sequence for the CMV enhancer;

SEQ ID NO: 107 is the nucleotide sequence for the CMV promoter;

SEQ ID NO: 108 is the nucleotide sequence for the SV40 NLS component;

SEQ ID NO: 109 is the nucleotide sequence for the T2A promoter;

SEQ ID NO: 110 is the nucleotide sequence for the smURFP reporter gene cassette;

SEQ ID NO: 111 is the nucleotide sequence for the poly-A-site;

SEQ ID NO: 112 is the nucleotide sequence for the 3′ AAV ITR component;

SEQ ID NO: 113 is the nucleotide sequence for the chimeric intron;

SEQ ID NO: 114 is the nucleotide sequence for the chimeric intron with SIN site 1;

SEQ ID NO: 115 is the nucleotide sequence for the chimeric intron with SIN site 2;

SEQ ID NO: 116 is the nucleotide sequence for the chimeric intron with a SIN site;

SEQ ID NO: 117 is the nucleotide sequence for the BCL11A intron 2;

SEQ ID NO: 118 is the nucleotide sequence for the BCL11A intron 2 with SIN site 1;

SEQ ID NO: 119 is the nucleotide sequence for the Retinoblastoma intron 16;

SEQ ID NO: 120 is the nucleotide sequence for the Retinoblastoma intron 16 with SIN site 1;

SEQ ID NOs: 121-138 are guide RNA nucleotide sequences used to generate the plasmid and AAV constructs.

### DETAILED DESCRIPTION

The CRISPR/Cas/Cpf1 system is a powerful tool for development of next generation medicines to treat/cure intractable, inherited and acquired diseases; however, sustained CRISPR/Cas9 or CRISPR/Cpf1 expression in a cell is no longer necessary once all copies of a gene in the genome of a cell of interest have been edited. Chronic and constitutive endonuclease activity of Cas9 or Cpf1 can increase the number of off-target mutations and/or can generate anti-Cas9 or anti-Cpf1 immune responses resulting in elimination of the gene edited cells. Thus, temporal- and/or spatial-limited expression of Cas9 or Cpf1 is desirable to reduce or eliminate unwanted off-target effects of the endonuclease activity of Cas9 or Cpf1. The spatiotemporal control of Cas9 or Cpf1 expression can be also executed to lower/eliminate immune responses to Cas9 or Cpf1 resulting in enhanced safety and efficacy of gene editing

### Terminology

All technical and scientific terms used herein have the same meaning as commonly understood by one of ordinary skill in the art to which this invention belongs, unless the technical or scientific term is defined differently herein.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and can be isolated from genes, or chemically synthesized by methods known in the art. The terms “polynucleotide” and “nucleic acid” should be understood to include, as applicable to the aspects being described, single-stranded (such as sense or antisense) and double-stranded polynucleotides.

“Genomic DNA” refers to the DNA of a genome of an organism including, but not limited to, the DNA of the genome of a bacterium, fungus, archea, plant or animal.

“Manipulating” DNA encompasses binding, nicking one strand, or cleaving (i.e., cutting) both strands of the DNA, or encompasses modifying the DNA or a polypeptide associated with the DNA. Manipulating DNA can silence, activate, or modulate (either increase or decrease) the expression of an RNA or polypeptide encoded by the DNA.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem can include one or more base mismatches. Alternatively, the base-pairing can be exact, i.e. not include any mismatches.

By “hybridizable” or “complementary” or “substantially complementary” it is meant that a nucleic acid (e.g. RNA) comprises a sequence of nucleotides that enables it to non-covalently bind, e.g.: form Watson-Crick base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA].

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides, through “seed sequences”. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration can be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide can hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides can be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction can be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻14 M, or less than 10⁻¹⁵ M. “Affinity” refers to the strength of binding, increased binding affinity being correlated with a lower K_{d}. By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.) and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, or mafft.cbrc.jp/alignment/software/. See, e.g., Altschul et al. (1990), J. Mol. Bioi. 215:403-10. Sequence alignments standard in the art are used according to the invention to determine amino acid residues in a Cas9 ortholog that “correspond to” amino acid residues in another Cas9 ortholog. The amino acid residues of Cas9 orthologs that correspond to amino acid residues of other Cas9 orthologs appear at the same position in alignments of the sequences.

A DNA sequence that “encodes” a particular RNA is a DNA nucleic acid sequence that is transcribed into RNA. A DNA polynucleotide can encode an RNA (mRNA) that is translated into protein, or a DNA polynucleotide can encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, can be used to drive the various vectors of the present invention.

A promoter can be a constitutively active promoter (i.e., a promoter that is constitutively in an active/“ON” state), it can be an inducible promoter (i.e., a promoter whose state, active/“ON” or inactive/“OFF”, is controlled by an external stimulus, e.g., the presence of a particular temperature, compound, or protein.), it can be a spatially restricted promoter (i.e., transcriptional control element, enhancer, etc.)(e.g., tissue specific promoter, cell type specific promoter, etc.), and it can be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., site-directed modifying polypeptide, or Cas9 polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to two components that are defined by structures derived from different sources. For example, where “chimeric” is used in the context of a chimeric polypeptide (e.g., a chimeric Cas9 protein), the chimeric polypeptide includes amino acid sequences that are derived from different polypeptides. A chimeric polypeptide can comprise either modified or naturally-occurring polypeptide sequences (e.g., a first amino acid sequence from a modified or unmodified Cas9 protein; and a second amino acid sequence other than the Cas9 protein). Similarly, “chimeric” in the context of a polynucleotide encoding a chimeric polypeptide includes nucleotide sequences derived from different coding regions (e.g., a first nucleotide sequence encoding a modified or unmodified Cas9 protein; and a second nucleotide sequence encoding a polypeptide other than a Cas9 protein).

The term “chimeric polypeptide” refers to a polypeptide which is not naturally occurring, e.g., is made by the artificial combination (i.e., “fusion”) of two otherwise separated segments of amino sequence through human intervention. A polypeptide that comprises a chimeric amino acid sequence is a chimeric polypeptide. Some chimeric polypeptides can be referred to as “fusion variants.”

“Heterologous,” as used herein, means a nucleotide or peptide that is not found in the native nucleic acid or protein, respectively. For example, in a chimeric Cas9 protein, the RNA-binding domain of a naturally-occurring bacterial Cas9 polypeptide (or a variant thereof) can be fused to a heterologous polypeptide sequence (i.e. a polypeptide sequence from a protein other than Cas9 or a polypeptide sequence from another organism). The heterologous polypeptide can exhibit an activity (e.g., enzymatic activity) that will also be exhibited by the chimeric Cas9 protein (e.g., methyltransferase activity, acetyltransferase activity, kinase activity, ubiquitinating activity, etc.). A heterologous nucleic acid can be linked to a naturally-occurring nucleic acid (or a variant thereof) (e.g., by genetic engineering) to generate a chimeric polynucleotide encoding a chimeric polypeptide. As another example, in a fusion variant Cas9 site-directed polypeptide, a variant Cas9 site-directed polypeptide can be fused to a heterologous polypeptide (i.e. a polypeptide other than Cas9), which exhibits an activity that will also be exhibited by the fusion variant Cas9 site-directed polypeptide. A heterologous nucleic acid can be linked to a variant Cas9 site-directed polypeptide (e.g., by genetic engineering) to generate a polynucleotide encoding a fusion variant Cas9 site-directed polypeptide. “Heterologous,” as used herein, additionally means a nucleotide or polypeptide in a cell that is not its native cell.

The term “cognate” refers to two biomolecules that normally interact or co-exist in nature.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA can be present 5′ or 3′ from the open reading frame, where such sequences do not interfere with manipulation or expression of the coding regions, and can indeed act to modulate production of a desired product by various mechanisms (see “DNA regulatory sequences”, below). Alternatively, DNA sequences encoding RNA (e.g., guide RNA) that is not translated can also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but can be a naturally occurring amino acid sequence.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) can or cannot be operably linked to a promoter sequence and can or cannot be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA can or cannot be integrated (covalently linked) into the genome of the cell.

In prokaryotes, yeast, and mammalian cells for example, the transforming DNA can be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell can not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a mammalian germ cell), by virtue of introduction into a suitable eukaryotic host cell of an exogenous nucleic acid.

A “target DNA” as used herein is a DNA polynucleotide that comprises a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment (e.g., spacer or spacer sequence) of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The target DNA can be a double-stranded DNA. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” By “site-directed modifying polypeptide” or “RNA-binding site-directed polypeptide” or “RNA-binding site-directed modifying polypeptide” or “site-directed polypeptide” it is meant a polypeptide that binds gRNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence). By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain aspects, a complex comprising a guide RNA and a site-directed modifying polypeptide is used for targeted double-stranded DNA cleavage.

A “self-inactivating site” or “SIN site” as used herein is a site within a self-inactivating vector that comprises a protospacer sequence and neighboring protospacer adjacent motif (PAM). For example, a SIN site can comprise 5′-N17-21NRG-3′ or 5′-N19-24NNGRRT-3′ wherein N17-21 or N19-24 represent protospacer sequence and NRG or NNGRRT represent PAMs for SpCas9 or SaCas9, respectively. The DNA targeting segment (e.g., spacer) of a DNA targeting nucleic acid (e.g., gRNA) hybridizes to the complementary strand of the protospacer sequence of the SIN site.

In certain aspects, the DNA targeting segment of the DNA targeting nucleic acid can be completely complementary to, and hybridize with the SIN site. In certain aspects, the SIN site can be substantially complementary, for example, having 1 or more mismatches, to the DNA targeting segment of the DNA targeting nucleic acid to modulate timing of self-inactivation.

In some aspects, the SIN site can comprise a PAM sequence for S. aureus Cas9, S. pyogenes Cas9, T. denticola Cas9, N. menginitidis Cas9, Cpf1, C. jejuni Cas9, S. thermophilus Cas9 or other orthologs described herein. In certain aspects the PAM sequence may be: NNGRRT, NRG, NAAAAN, NAAAAC, NNNNGHTT, YTN, NNNNACA, NNNACAC, NNVRYAC, NNNVRYM, NNAAAAW, or NNAGAAW.

“Nuclease” and “endonuclease” are used interchangeably herein to mean an enzyme which possesses endonucleolytic catalytic activity for DNA cleavage.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain can consist of more than one isolated stretch of amino acids within a given polypeptide.

By “site-directed polypeptide” or “RNA-binding site-directed polypeptide” or “RNA-binding site-directed modifying polypeptide” it is meant a polypeptide that binds RNA and is targeted to a specific DNA sequence. A site-directed polypeptide as described herein is targeted to a specific DNA sequence by the RNA molecule to which it is bound. The RNA molecule comprises a sequence that is complementary to a target sequence within the target DNA, thus targeting the bound polypeptide to a specific location within the target DNA (the target sequence).

The RNA molecule that binds to the site-directed modifying polypeptide and targets the polypeptide to a specific location within the target DNA is referred to herein as the “guide RNA” or “guide RNA polynucleotide” (also referred to herein as a “guide RNA” or “gRNA”). A guide RNA comprises two segments, a “DNA-targeting segment” and a “protein-binding segment.” By “segment” it is meant a segment/section/region of a molecule, e.g., a contiguous stretch of nucleotides in an RNA. A segment can also mean a region/section of a complex such that a segment can comprise regions of more than one molecule. For example, in some cases the protein-binding segment (described below) of a guide RNA is one RNA molecule and the protein-binding segment therefore comprises a region of that RNA molecule. In other cases, the protein-binding segment (described below) of a guide RNA comprises two separate molecules that are hybridized along a region of complementarity. As an illustrative, non-limiting example, a protein-binding segment of a guide RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can or cannot include regions with complementarity to other molecules.

The DNA-targeting segment (or “DNA-targeting sequence”) comprises a nucleotide sequence that is complementary to a specific sequence within a target DNA (the complementary strand of the target DNA) designated the “protospacer-like” sequence herein. The DNA-targeting segment of a gRNA is also referred to as the spacer or spacer sequence herein. The protein-binding segment (or “protein-binding sequence”) interacts with a site-directed modifying polypeptide. When the site-directed modifying polypeptide is a Cas9, Cas9 related polypeptide, Cpf1, or Cpf1 related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA.

The protein-binding segment of a guide RNA comprises, in part, two complementary stretches of nucleotides that hybridize to one another to form a double stranded RNA duplex (dsRNA duplex).

In some examples, a nucleic acid (e.g., a guide RNA, a nucleic acid comprising a nucleotide sequence encoding a guide RNA; a nucleic acid encoding a site-directed polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some examples, a guide RNA comprises an additional segment at either the 5′ or 3′ end that provides for any of the features described above. For example, a suitable third segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guide RNA and a site-directed modifying polypeptide (i.e., site-directed polypeptide) form a complex (i.e., bind via non-covalent interactions). The guide RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

In some examples, a guide RNA comprises two separate RNA molecules (RNA polynucleotides: an “activator-RNA” and a “targeter-RNA”, see below) and is referred to herein as a “double-molecule guide RNA” or a “two-molecule guide RNA.” In other examples, the guide RNA is a single RNA molecule (single RNA polynucleotide) and is referred to herein as a “single-molecule guide RNA,” a “single-guide RNA,” or an “sgRNA.” The term “guide RNA” or “gRNA” is inclusive, referring both to double-molecule guide RNAs (also called a “split guide”) and to single-molecule guide RNAs (i.e., sgRNAs).

A two-molecule guide RNA comprises two separate RNA molecules (a “targeter-RNA” and an “activator-RNA”). Each of the two RNA molecules of a two-molecule guide RNA comprises a stretch of nucleotides that are complementary to one another such that the complementary nucleotides of the two RNA molecules hybridize to form the double stranded RNA duplex of the protein-binding segment.

An exemplary two-molecule guide RNA comprises a crRNA-like (“CRISPR RNA” or “targeter-RNA”) molecule (which includes a CRISPR repeat or CRISPR repeat-like sequence) and a corresponding tracrRNA-like (“trans-activating CRISPR RNA” or “activator-RNA” or “tracrRNA”) molecule. A crRNA-like molecule (targeter-RNA) comprises both the DNA-targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator-RNA) comprises a stretch of nucleotides (duplex-forming segment) that forms the other half of the dsRNA duplex of the protein-binding segment of the guide RNA. In other words, a stretch of nucleotides of a crRNA-like molecule are complementary to and hybridize with a stretch of nucleotides of a tracrRNA-like molecule to form the dsRNA duplex of the protein-binding domain of the guide RNA. As such, each crRNA-like molecule can be said to have a corresponding tracrRNA-like molecule. The crRNA-like molecule additionally provides the single stranded DNA-targeting segment. Thus, a crRNA-like and a tracrRNA-like molecule (as a corresponding pair) hybridize to form a guide RNA. A double-molecule guide RNA can comprise any corresponding crRNA and tracrRNA pair.

A two-molecule guide RNA can be designed to allow for controlled (i.e., conditional) binding of a targeter-RNA with an activator-RNA. Because a two-molecule guide RNA is not functional unless both the activator-RNA and the targeter-RNA are bound in a functional complex with Cas9, a two-molecule guide RNA can be inducible (e.g., drug inducible) by rendering the binding between the activator-RNA and the targeter-RNA to be inducible. As one non-limiting example, RNA aptamers can be used to regulate (i.e., control) the binding of the activator-RNA with the targeter-RNA. Accordingly, the activator-RNA and/or the targeter-RNA can comprise an RNA aptamer sequence.

A single-molecule guide RNA comprises two stretches of nucleotides (a targeter-RNA and an activator-RNA) that are complementary to one another, are covalently linked (directly, or by intervening nucleotides), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The term “activator-RNA” is used herein to mean a tracrRNA-like molecule of a double-molecule guide RNA. The term “targeter-RNA” is used herein to mean a crRNA-like molecule of a double-molecule guide RNA. The term “duplex-forming segment” is used herein to mean the stretch of nucleotides of an activator-RNA or a targeter-RNA that contributes to the formation of the dsRNA duplex by hybridizing to a stretch of nucleotides of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the DNA-targeting segment of the guide RNA. Therefore, a double-molecule guide RNA can be comprised of any corresponding activator-RNA and targeter-RNA pair.

RNA aptamers are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the RNA molecule of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator-RNA with an aptamer cannot be able to bind to the cognate targeter-RNA unless the aptamer is bound by the appropriate drug; (ii) a targeter-RNA with an aptamer cannot be able to bind to the cognate activator-RNA unless the aptamer is bound by the appropriate drug; and (iii) a targeter-RNA and an activator-RNA, each comprising a different aptamer that binds a different drug, cannot be able to bind to each other unless both drugs are present. As illustrated by these examples, a two-molecule guide RNA can be designed to be inducible.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell. PSCs can be target cells of the methods described herein.

By “embryonic stem cell” (ESC) is meant a PSC that was isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs can be found in, for example, U.S. Pat. Nos. 7,029,913, 5,843,780, and 6,200,806, the disclosures of which are incorporated herein by reference. Methods for proliferating hESCs in the undifferentiated form are described in WO 99/20741, WO 01/51616, and WO 03/020920. By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells can be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs can be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which can be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e. it is no longer undergoing divisions. This quiescent state can be temporary, i.e. reversible, or it can be permanent.

By “meiotic cell” it is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair can result in an alteration of the sequence of the target molecule (e.g., insertion, deletion, mutation), if the donor polynucleotide differs from the target molecule and part or all of the sequence of the donor polynucleotide is incorporated into the target DNA. In some examples, the donor polynucleotide, a portion of the donor polynucleotide, a copy of the donor polynucleotide, or a portion of a copy of the donor polynucleotide integrates into the target DNA.

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The terms “treatment”, “treating” and the like are used herein to generally mean obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which can be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent can be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

The term “comprising” or “comprises” is used in reference to compositions, methods, and respective component(s) thereof, that are essential to the present disclosure, yet open to the inclusion of unspecified elements, whether essential or not.

The term “consisting essentially of” refers to those elements required for a given aspect. The term permits the presence of additional elements that do not materially affect the basic and novel or functional characteristic(s) of that aspect of the present disclosure.

The term “consisting of” refers to compositions, methods, and respective components thereof as described herein, which are exclusive of any element not recited in that description of the aspect.

Any numerical range recited in this specification describes all sub-ranges of the same numerical precision (i.e., having the same number of specified digits) subsumed within the recited range. For example, a recited range of “1.0 to 10.0” describes all sub-ranges between (and including) the recited minimum value of 1.0 and the recited maximum value of 10.0, such as, for example, “2.4 to 7.6,” even if the range of “2.4 to 7.6” is not expressly recited in the text of the specification. Accordingly, the Applicant reserves the right to amend this specification, including the claims, to expressly recite any sub-range of the same numerical precision subsumed within the ranges expressly recited in this specification. All such ranges are inherently described in this specification such that amending to expressly recite any such sub-ranges will comply with written description, sufficiency of description, and added matter requirements, including the requirements under 35 U.S.C. § 112(a) and Article 123(2) EPC. Also, unless expressly specified or otherwise required by context, all numerical parameters described in this specification (such as those expressing values, ranges, amounts, percentages, and the like) may be read as if prefaced by the word “about,” even if the word “about” does not expressly appear before a number. Additionally, numerical parameters described in this specification should be construed in light of the number of reported significant digits, numerical precision, and by applying ordinary rounding techniques. It is also understood that numerical parameters described in this specification will necessarily possess the inherent variability characteristic of the underlying measurement techniques used to determine the numerical value of the parameter.

It is appreciated that certain features of the invention, which are, for clarity, described in the context of separate examples, can also be provided in combination in a single example. Conversely, various features of the invention, which are, for brevity, described in the context of a single example, can also be provided separately or in any suitable sub-combination. All combinations of the examples pertaining to the disclosure are specifically embraced by the present invention and are disclosed herein just as if each and every combination was individually and explicitly disclosed. In addition, all sub-combinations of the various examples and elements thereof are also specifically embraced by the present invention and are disclosed herein just as if each and every such sub-combination was individually and explicitly disclosed herein.

### CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, biogenesis of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary structures (e.g. hairpin structures) and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. Some Cas genes comprise homologous secondary and/or tertiary structures.

### Type II CRISPR Systems

crRNA biogenesis in a Type II CRISPR system in nature requires a trans-activating CRISPR RNA (tracrRNA). The tracrRNA can be modified by endogenous RNaseIII, and then hybridizes to a crRNA repeat in the pre-crRNA array. Endogenous RNaseIII can be recruited to cleave the pre-crRNA. Cleaved crRNAs can be subjected to exoribonuclease trimming to produce the mature crRNA form (e.g., 5′ trimming). The tracrRNA can remain hybridized to the crRNA, and the tracrRNA and the crRNA associate with a site-directed polypeptide (e.g., Cas9). The crRNA of the crRNA-tracrRNA-Cas9 complex can guide the complex to a target nucleic acid to which the crRNA can hybridize. Hybridization of the crRNA to the target nucleic acid can activate Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096):816-821 (2012) showed that the CRISPR/Cas9 system is useful for RNA-programmable genome editing, and international patent application publication number WO2013/176772 provides numerous examples and applications of the CRISPR/Cas endonuclease system for site-specific gene editing.

### Type V CRISPR Systems

Type V CRISPR systems have several important differences from Type II systems. For example, Cpf1 is a single RNA-guided endonuclease that, in contrast to Type II systems, lacks tracrRNA. In fact, Cpf1-associated CRISPR arrays can be processed into mature crRNAs without the requirement of an additional trans-activating tracrRNA. The Type V CRISPR array can be processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems can start with 20-24 nucleotides of spacer sequence followed by about 22 nucleotides of direct repeat. Also, Cpf1 can utilize a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

### Cas Genes/Polypeptides and Protospacer Adjacent Motifs

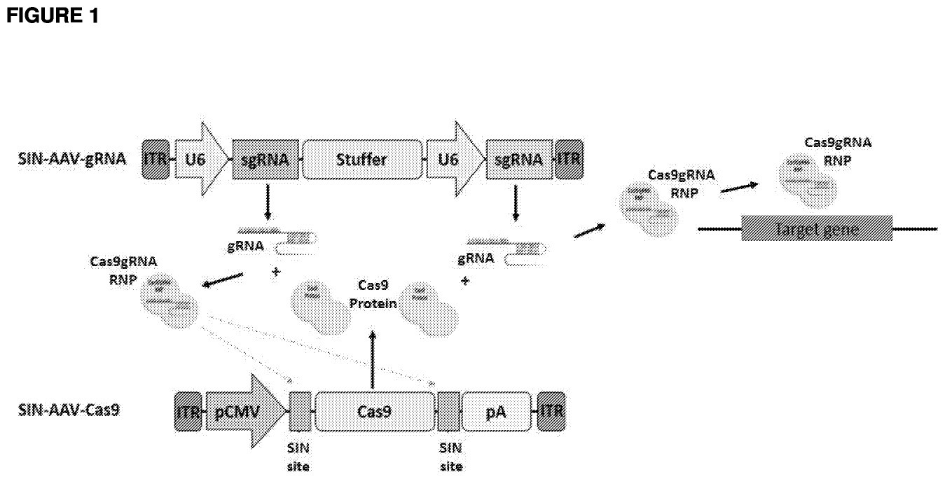

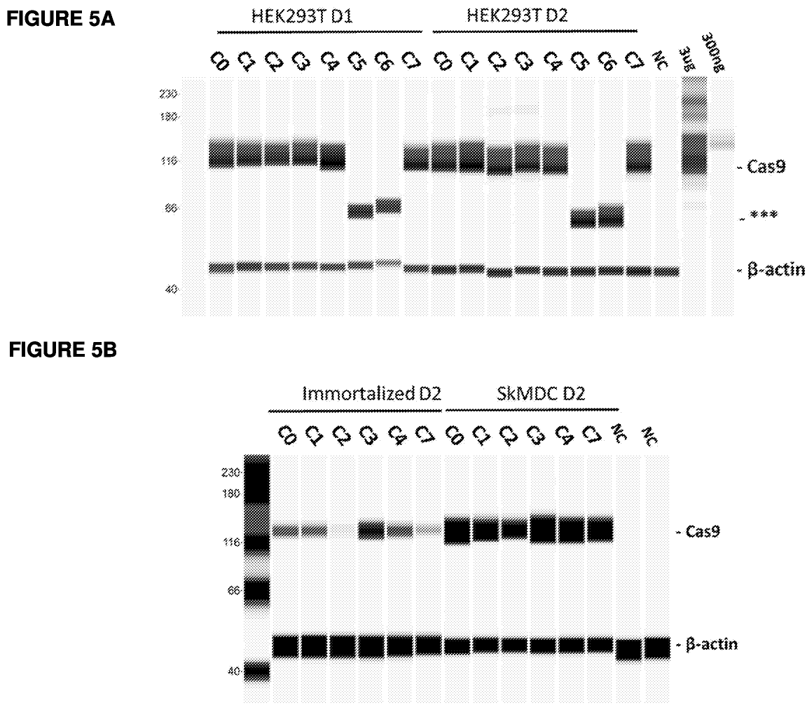

Exemplary CRISPR/Cas polypeptides include the Cas9 polypeptides in FIG. 1 of Fonfara et al., Nucleic Acids Research, 42: 2577-2590 (2014). The CRISPR/Cas gene naming system has undergone extensive rewriting since the Cas genes were discovered. FIG. 5 of Fonfara, supra, provides PAM sequences for the Cas9 polypeptides from various species. Additional PAM sequences include, but are not limited to, S. aureus PAM sequence NNGRRT, S. pyogenes PAM sequence NRG, T. denticola PAM sequence NAAAAN or NAAAAC, N. menginitidis PAM sequence NNNNGHTT, Cpf1 PAM sequence YTN, C. jejuni PAM sequence NNNNACA, NNNACAC, NNVRYAC, or NNNVRYM; P. multocida PAM sequences GNNNCNNA or NNNNC; an F. novicida PAM sequence NG; an S. thermophilus PAM sequences NNAAAAW and NNAGAAW; an L. innocua PAM sequence NGG; and an S. dysgalactiae PAM sequence NGG.

### Site-Directed Polypeptides

A site-directed polypeptide is a nuclease used in genome editing to cleave DNA. The site-directed polypeptide can be administered to a cell or a patient as either: one or more polypeptides, or one or more mRNAs encoding the polypeptide.

In the context of a CRISPR/Cas or CRISPR/Cpf1 system, the site-directed polypeptide can bind to a guide RNA that, in turn, specifies the site in the target DNA to which the polypeptide is directed. In the CRISPR/Cas or CRISPR/Cpf1 systems herein, the site-directed polypeptide can be an endonuclease, such as a DNA endonuclease.

A site-directed polypeptide can comprise a plurality of nucleic acid-cleaving (i.e., nuclease) domains. Two or more nucleic acid-cleaving domains can be linked together via a linker. For example, the linker can comprise a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

Naturally-occurring wild-type Cas9 enzymes comprise two nuclease domains, a HNH nuclease domain and a RuvC domain. Herein, the “Cas9” refers to both naturally-occurring and recombinant Cas9s. Cas9 enzymes contemplated herein can comprise a HNH or HNH-like nuclease domain, and/or a RuvC or RuvC-like nuclease domain.

HNH or HNH-like domains comprise a McrA-like fold. HNH or HNH-like domains comprises two antiparallel β-strands and an a-helix. HNH or HNH-like domains comprises a metal binding site (e.g., a divalent cation binding site). HNH or HNH-like domains can cleave one strand of a target nucleic acid (e.g., the complementary strand of the crRNA targeted strand).

RuvC or RuvC-like domains comprise an RNaseH or RNaseH-like fold. RuvC/RNaseH domains are involved in a diverse set of nucleic acid-based functions including acting on both RNA and DNA. The RNaseH domain comprises 5 β-strands surrounded by a plurality of a-helices. RuvC/RNaseH or RuvC/RNaseH-like domains comprise a metal binding site (e.g., a divalent cation binding site). RuvC/RNaseH or RuvC/RNaseH-like domains can cleave one strand of a target nucleic acid (e.g., the non-complementary strand of a double-stranded target DNA).

Site-directed polypeptides can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining (NHEJ) or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage, and can lead to disruption or alteration of gene expression. HDR can occur when a homologous repair template, or donor, is available. The homologous donor template can comprise sequences that are homologous to sequences flanking the target nucleic acid cleavage site. The sister chromatid can be used by the cell as the repair template. However, for the purposes of genome editing, the repair template can be supplied as an exogenous nucleic acid, such as a plasmid, duplex oligonucleotide, single-strand oligonucleotide or viral nucleic acid. With exogenous donor templates, an additional nucleic acid sequence (such as a transgene) or modification (such as a single or multiple base change or a deletion) can be introduced between the flanking regions of homology so that the additional or altered nucleic acid sequence also becomes incorporated into the target locus. MMEJ can result in a genetic outcome that is similar to NHEJ in that small deletions and insertions can occur at the cleavage site. MMEJ can make use of homologous sequences of a few base pairs flanking the cleavage site to drive a favored end-joining DNA repair outcome. In some instances it can be possible to predict likely repair outcomes based on analysis of potential microhomologies in the nuclease target regions.

Thus, in some cases, homologous recombination can be used to insert an exogenous polynucleotide sequence into the target nucleic acid cleavage site. An exogenous polynucleotide sequence is termed a donor polynucleotide (or donor or donor sequence) herein. The donor polynucleotide, a portion of the donor polynucleotide, a copy of the donor polynucleotide, or a portion of a copy of the donor polynucleotide can be inserted into the target nucleic acid cleavage site. The donor polynucleotide can be an exogenous polynucleotide sequence, i.e., a sequence that does not naturally occur at the target nucleic acid cleavage site.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

The site-directed polypeptide can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US2014/0068797 Sequence ID No. 8 or Sapranauskas et al., Nucleic Acids Res, 39(21): 9275-9282 (2011), or Cas9 from S.aureus, WO2015/071474 Sequence ID No. 244], and various other site-directed polypeptides.

The site-directed polypeptide can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes or S. aureus, supra).

The site-directed polypeptide can comprise at least 70, 75, 80, 85, 90, 95, 97, 99, or 100% identity to a wild-type site-directed polypeptide (e.g., Cas9 from S. pyogenes or S. aureus, supra) over 10 contiguous amino acids. The site-directed polypeptide can comprise at most: 70, 75, 80, 85, 90, 95, 97, 99, or 100% identity to a wild-type site-directed polypeptide (e.g., Cas9 from S. pyogenes or S. aureus, supra) over 10 contiguous amino acids. The site-directed polypeptide can comprise at least: 70, 75, 80, 85, 90, 95, 97, 99, or 100% identity to a wild-type site-directed polypeptide (e.g., Cas9 from S. pyogenes or S. aureus, supra) over 10 contiguous amino acids in a HNH nuclease domain of the site-directed polypeptide. The site-directed polypeptide can comprise at most: 70, 75, 80, 85, 90, 95, 97, 99, or 100% identity to a wild-type site-directed polypeptide (e.g., Cas9 from S. pyogenes or S. aureus, supra) over 10 contiguous amino acids in a HNH nuclease domain of the site-directed polypeptide. The site-directed polypeptide can comprise at least: 70, 75, 80, 85, 90, 95, 97, 99, or 100% identity to a wild-type site-directed polypeptide (e.g., Cas9 from S. pyogenes or S. aureus, supra) over 10 contiguous amino acids in a RuvC nuclease domain of the site-directed polypeptide. The site-directed polypeptide can comprise at most: 70, 75, 80, 85, 90, 95, 97, 99, or 100% identity to a wild-type site-directed polypeptide (e.g., Cas9 from S. pyogenes or S. aureus, supra) over 10 contiguous amino acids in a RuvC nuclease domain of the site-directed polypeptide.

The site-directed polypeptide can comprise a modified form of a wild-type exemplary site-directed polypeptide. The modified form of the wild-type exemplary site-directed polypeptide can comprise a mutation that reduces the nucleic acid-cleaving activity of the site-directed polypeptide. The modified form of the wild-type exemplary site-directed polypeptide can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes or S. aureus, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

The modified form of the site-directed polypeptide can comprise a mutation such that it can induce a single-strand break (SSB) on a target nucleic acid (e.g., by cutting only one of the sugar-phosphate backbones of a double-strand target nucleic acid). The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes or S. aureus, supra). The mutation can result in one or more of the plurality of nucleic acid-cleaving domains retaining the ability to cleave the complementary strand of the target nucleic acid, but reducing its ability to cleave the non-complementary strand of the target nucleic acid. The mutation can result in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854 and Asn856, are mutated to inactivate one or more of the plurality of nucleic acid-cleaving domains (e.g., nuclease domains). The residues to be mutated can correspond to residues Asp10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. Additional examples of mutations can include N497A, R661A, N692A, M694A, Q695A, H698A, E762A, K810A, K848A, K855A, N863A, Q926A, D986A, K1003A and R1060A. One skilled in the art will recognize that mutations other than alanine substitutions can be suitable.

A D10A mutation can be combined with one or more of H840A, N854A, or N856A mutations to produce a site-directed polypeptide substantially lacking DNA cleavage activity. A H840A mutation can be combined with one or more of D10A, N854A, or N856A mutations to produce a site-directed polypeptide substantially lacking DNA cleavage activity. A N854A mutation can be combined with one or more of H840A, D10A, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In another example, residues in the wild-type exemplary S. aureus Cas9 polypeptide, such as Asp10 or Asn580 are mutated to inactivate one or more of the plurality of nucleic acid-cleaving domains (e.g., nuclease domains). Non-limiting examples of mutations include D10A and N580A. A D10A mutation can be combined with one or more mutations, including N580A to produce a site-directed polypeptide substantially lacking DNA cleavage activity.

Site-directed polypeptides that comprise one substantially inactive nuclease domain are referred to as “nickases”.

Nickase variants of RNA-guided endonucleases, for example Cas9, can be used to increase the specificity of CRISPR-mediated genome editing. Wild type Cas9 is typically guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes.

Mutations contemplated can include substitutions, additions, and deletions, or any combination thereof. The mutation converts the mutated amino acid to alanine. The mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagines, glutamine, histidine, lysine, or arginine). The mutation converts the mutated amino acid to a non-natural amino acid (e.g., selenomethionine). The mutation converts the mutated amino acid to amino acid mimics (e.g., phosphomimics). The mutation can be a conservative mutation. For example, the mutation can convert the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation). The mutation can cause a shift in reading frame and/or the creation of a premature stop codon. Mutations can cause changes to regulatory regions of genes or loci that affect expression of one or more genes.

The site-directed polypeptide (e.g., variant, mutated, enzymatically inactive and/or conditionally enzymatically inactive site-directed polypeptide) can target nucleic acid. The site-directed polypeptide (e.g., variant, mutated, enzymatically inactive and/or conditionally enzymatically inactive endoribonuclease) can target DNA. The site-directed polypeptide (e.g. variant, mutated, enzymatically inactive and/or conditionally enzymatically inactive endoribonuclease) can target RNA.

The site-directed polypeptide can comprise one or more non-native sequences (e.g., the site-directed polypeptide is a fusion protein).

The site-directed polypeptide can comprise an amino acid sequence comprising at least 15% amino acid identity to a Cas9 from a bacterium (e.g., S. pyogenes or S. aureus), a nucleic acid binding domain, and two nucleic acid cleaving domains (i.e., a HNH domain and a RuvC domain).

The site-directed polypeptide can comprise an amino acid sequence comprising at least 15% amino acid identity to a Cas9 from a bacterium (e.g., S. pyogenes or S. aureus), and two nucleic acid cleaving domains (i.e., a HNH domain and a RuvC domain).

The site-directed polypeptide can comprise an amino acid sequence comprising at least 15% amino acid identity to a Cas9 from a bacterium (e.g., S. pyogenes or S. aureus), and two nucleic acid cleaving domains, wherein one or both of the nucleic acid cleaving domains comprise at least 50% amino acid identity to a nuclease domain from Cas9 from a bacterium (e.g., S. pyogenes).

The site-directed polypeptide can comprise an amino acid sequence comprising at least 15% amino acid identity to a Cas9 from a bacterium (e.g., S. pyogenes or S. aureus), two nucleic acid cleaving domains (i.e., a HNH domain and a RuvC domain), and non-native sequence (for example, a nuclear localization signal) or a linker linking the site-directed polypeptide to a non-native sequence.

The site-directed polypeptide can comprise an amino acid sequence comprising at least 15% amino acid identity to a Cas9 from a bacterium (e.g., S. pyogenes or S. aureus), two nucleic acid cleaving domains (i.e., a HNH domain and a RuvC domain), wherein the site-directed polypeptide comprises a mutation in one or both of the nucleic acid cleaving domains that reduces the cleaving activity of the nuclease domains by at least 50%.

The site-directed polypeptide can comprise an amino acid sequence comprising at least 15% amino acid identity to a Cas9 from a bacterium (e.g., S. pyogenes or S. aureus), and two nucleic acid cleaving domains (i.e., a HNH domain and a RuvC domain), wherein one of the nuclease domains comprises mutation of aspartic acid 10, and/or wherein one of the nuclease domains can comprise a mutation of histidine 840, and/or wherein one of the nuclease domains can comprise a mutation of Asparagine 580 and wherein the mutation reduces the cleaving activity of the nuclease domain(s) by at least 50%.

The one or more site-directed polypeptides, e.g. DNA endonucleases, can comprise two nickases that together effect one double-strand break at a specific locus in the genome, or four nickases that together effect or cause two double-strand breaks at specific loci in the genome. Alternatively, one site-directed polypeptide, e.g. DNA endonuclease, can effect or cause one double-strand break at a specific locus in the genome.

### DNA-Targeting Nucleic Acid

The present disclosure provides a DNA-targeting nucleic acid that can direct the activities of an associated polypeptide (e.g., a site-directed polypeptide) to a specific target sequence within a target nucleic acid. The DNA-targeting nucleic acid can target genomic DNA. A DNA-targeting nucleic acid that targets genomic DNA may be referred to as a genomic-targeting nucleic acid. In addition, the DNA-targeting nucleic acid can target a vector, a plasmid, a viral vector, an AAV, or an expression vector. The DNA-targeting nucleic acid can target SIN sites. The DNA-targeting nucleic acid can be RNA. A DNA-targeting RNA is referred to as a “guide RNA” or “gRNA” herein. A guide RNA or gRNA can be genomic-targeting RNA. A guide RNA can comprise at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex can bind a site-directed polypeptide, such that the guide RNA and site-direct polypeptide form a complex. The DNA-targeting nucleic acid can provide target specificity to the complex by virtue of its association with the site-directed polypeptide. The DNA-targeting nucleic acid can direct the activity of the site-directed polypeptide.

The DNA-targeting nucleic acid can be a double-molecule guide RNA. The DNA-targeting nucleic acid can be a single-molecule guide RNA.

A double-molecule guide RNA can comprise two strands of RNA. The first strand comprises in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

A single-molecule guide RNA (sgRNA) in a Type II system can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

The sgRNA can comprise a 20 nucleotide spacer sequence at the 5′ end of the sgRNA sequence. The sgRNA can comprise a less than a 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence (see Table 1).

The sgRNA can comprise no uracil at the 3′end of the sgRNA sequence, such as in SEQ ID NOs: 8 and 10-11 of Table 1. The sgRNA can comprise one or more uracil at the 3′end of the sgRNA sequence, such as in SEQ ID NOs: 7, 9, and 12-15 in Table 1. For example, the sgRNA can comprise 1 uracil (U) at the 3′end of the sgRNA sequence. The sgRNA can comprise 2 uracil (UU) at the 3′end of the sgRNA sequence. The sgRNA can comprise 3 uracil (UUU) at the 3′end of the sgRNA sequence. The sgRNA can comprise 4 uracil (UUUU) at the 3′end of the sgRNA sequence. The sgRNA can comprise 5 uracil (UUUUU) at the 3′end of the sgRNA sequence. The sgRNA can comprise 6 uracil (UUUUUU) at the 3′end of the sgRNA sequence. The sgRNA can comprise 7 uracil (UUUUUUU) at the 3′end of the sgRNA sequence. The sgRNA can comprise 8 uracil (UUUUUUUU) at the 3′end of the sgRNA sequence.

The sgRNA can be unmodified or modified. For example, modified sgRNAs can comprise one or more 2′-O-methyl phosphorothioate nucleotides.

TABLE 1

| SEQ ID | |
|---|---|
| NO. | sgRNA sequence |
| 7 | nnnnnnnnnnnnnnnnnnnnguuuuagagcuagaaauagcaaguuaaaauaaggcuaguccguuauc |
| | aacuugaaaaaguggcaccgagucggugcuuuu |
| 8 | nnnnnnnnnnnnnnnnnnnnguuuuagagcuagaaauagcaaguuaaaauaaggcuaguccguuauc |
| | aacuugaaaaaguggcaccgagucggugc |
| 9 | n₍₁₇₋₃₀₎guuuuagagcuagaaauagcaaguuaaaauaaggcuaguccguuaucaacuugaaaaagu |
| | ggcaccgagucggugcu₍₁₋₈₎ |
| 10 | n₍₂₀₎guuuuaguacucuguaaugaaaauuacagaaucuacuaaaacaaggcaaaaugccguguuuaucu |
| | cgucaacuuguuggcgaga |
| 11 | n₍₂₀₎guuuaaguacucugugcuggaaacagcacagaaucuacuuaaacaaggcaaaaugccguguuuau |
| | cucgucaacuuguuggcgaga |
| 12 | n₍₂₀₎guuuuaguacucuguaaugaaaauuacagaaucuacuaaaacaaggcaaaaugccguguuuaucu |
| | cgucaacuuguuggcgagau₍₇₎ |
| 13 | n₍₂₀₎guuuaaguacucugugcuggaaacagcacagaaucuacuuaaacaaggcaaaaugccguguuuau |
| | cucgucaacuuguuggcgagau₍₇₎ |
| 14 | n₍₁₇₋₃₀₎guuuuaguacucuguaaugaaaauuacagaaucuacuaaaacaaggcaaaaugccguguuu |
| | aucucgucaacuuguuggcgagau₍₁₋₈₎ |
| 15 | n₍₁₇₋₃₀₎guuuaaguacucugugcuggaaacagcacagaaucuacuuaaacaaggcaaaaugccguguuu |
| | aucucgucaacuuguuggcgagau₍₁₋₈₎ |

A single-molecule guide RNA (sgRNA) in a Type V system can comprise, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacer Extension Sequence

In some examples of DNA-targeting nucleic acids, a spacer extension sequence can modify activity, provide stability and/or provide a location for modifications of a DNA-targeting nucleic acid. A spacer extension sequence can modify on- or off-target activity or specificity. In some examples, a spacer extension sequence can be provided. The spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. The spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. The spacer extension sequence can be less than 10 nucleotides in length. The spacer extension sequence can be between 10-30 nucleotides in length. The spacer extension sequence can be between 30-70 nucleotides in length.

The spacer extension sequence can comprise another moiety (e.g., a stability control sequence, an endoribonuclease binding sequence, a ribozyme). The moiety can increase or decrease the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

### Spacer Sequence

The spacer sequence hybridizes to a sequence in a target nucleic acid of interest. The spacer of a DNA-targeting nucleic acid can interact with a target nucleic acid in a sequence-specific manner via hybridization (i.e., base pairing). The nucleotide sequence of the spacer can vary depending on the sequence of the target nucleic acid of interest. The spacer sequence is also referred to as the DNA-targeting segment.

In a CRISPR/Cas or CRISPR/Cpf1 system disclosed herein, the spacer sequence can be designed to hybridize to a target sequence that is located 5′ of a PAM of the Cas9 or Cpf1 enzyme used in the system. The spacer can perfectly match the target sequence or can have mismatches. Each Cas9 enzyme has a particular PAM sequence that it recognizes in a target DNA. For example, S. pyogenes Cas9 recognizes in a target nucleic acid a PAM that comprises the sequence 5′-NRG-3′, where R comprises either A or G, where N is any nucleotide and N is immediately 3′ of the target nucleic acid sequence targeted by the spacer sequence. For example, S. aureus Cas9 recognizes in a target nucleic acid a PAM that comprises the sequence 5′-NNGRRT-3′, where R comprises either A or G, where N is any nucleotide and N is immediately 3′ of the target nucleic acid sequence targeted by the spacer sequence. In certain examples, S. aureus Cas9 recognizes in a target nucleic acid a PAM that comprises the sequence 5′-NNGRRN-3′, where R comprises either A or G, where N is any nucleotide and the N is immediately 3′ of the target nucleic acid sequence targeted by the spacer sequence. For example, C. jejuni recognizes in a target nucleic acid a PAM that comprises the sequence 5′-NNNNACA-3′ or 5′-NNNNACAC-3′, where N is any nucleotide and N is immediately 3′ of the target nucleic acid sequence targeted by the spacer sequence. In certain examples, C. jejuni Cas9 recognizes in a target nucleic acid a PAM that comprises the sequence 5′-NNNVRYM-3′ or 5′-NNVRYAC-3′, where V comprises either A, G or C, where R comprises either A or G, where Y comprises either C or T, where M comprises A or C, where N is any nucleotide and the N is immediately 3′ of the target nucleic acid sequence targeted by the spacer sequence.

The target nucleic acid sequence can comprise 20 nucleotides. The target nucleic acid can comprise less than 20 nucleotides. The target nucleic acid can comprise more than 20 nucleotides. The target nucleic acid can comprise at least: 5, 10, 15, 16, 17, 18, 19, 20, 21, 22, 23, 24, 25, 30 or more nucleotides. The target nucleic acid can comprise at most: 5, 10, 15, 16, 17, 18, 19, 20, 21, 22, 23, 24, 25, 30 or more nucleotides. The target nucleic acid sequence can comprise 20 bases immediately 5′ of the first nucleotide of the PAM. For example, in a sequence comprising 5′-NNNNNNNNNNNNNNNNNNNNNRG-3′, (SEQ ID NO: 28) the target nucleic acid can comprise the sequence that corresponds to the Ns, wherein N is any nucleotide, and the underlined NRG sequence is the S. pyogenes PAM. The target nucleic acid sequence can comprise 21 bases immediately 5′ of the first nucleotide of the PAM. For example, in a sequence comprising 5′-NNNNNNNNNNNNNNNNNNNNNNRG-3′, (SEQ ID NO: 29) the target nucleic acid can comprise the sequence that corresponds to the Ns, wherein N is any nucleotide, and the underlined NRG sequence is the S. pyogenes PAM. The target nucleic acid sequence can comprise 20 bases immediately 5′ of the first nucleotide of the PAM. For example, in a sequence comprising 5′-NNNNNNNNNNNNNNNNNNNNNNGRRT-3′, (SEQ ID NO: 30) the target nucleic acid can comprise the sequence that corresponds to the Ns, wherein N is any nucleotide, and the underlined NNGRRT sequence is the S. aureus PAM. The target nucleic acid sequence can comprise 21 bases immediately 5′ of the first nucleotide of the PAM. For example, in a sequence comprising 5′-NNNNNNNNNNNNNNNNNNNNNNNGRRT-3′, (SEQ ID NO: 31) the target nucleic acid can comprise the sequence that corresponds to the Ns, wherein N is any nucleotide, and the underlined NNGRRT sequence is the S. aureus PAM. The target nucleic acid sequence can comprise 20 bases immediately 5′ of the first nucleotide of the PAM. For example, in a sequence comprising 5′-NNNNNNNNNNNNNNNNNNNNNNGRRN-3′, (SEQ ID NO: 32) the target nucleic acid can comprise the sequence that corresponds to the Ns, wherein N is any nucleotide, and the underlined NNGRRN sequence is the S. aureus PAM. The target nucleic acid sequence can comprise 21 bases immediately 5′ of the first nucleotide of the PAM. For example, in a sequence comprising 5′-NNNNNNNNNNNNNNNNNNNNNNNGRRN-3′, (SEQ ID NO: 33) the target nucleic acid can comprise the sequence that corresponds to the Ns, wherein N is any nucleotide, and the underlined NNGRRN sequence is the S. aureus PAM.

The spacer sequence that hybridizes to the target nucleic acid can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer can comprise 19 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which can be thought of as a bulge or bulges.

The spacer sequence can be designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

### Minimum CRISPR Repeat Sequence

A minimum CRISPR repeat sequence can be a sequence with at least about 30%, about 40%, about 50%, about 60%, about 65%, about 70%, about 75%, about 80%, about 85%, about 90%, about 95%, or 100% sequence identity to a reference CRISPR repeat sequence (e.g., crRNA from S. pyogenes or S. aureus).

A minimum CRISPR repeat sequence can comprise nucleotides that can hybridize to a minimum tracrRNA sequence in a cell. The minimum CRISPR repeat sequence and a minimum tracrRNA sequence can form a duplex, i.e. a base-paired double-stranded structure. Together, the minimum CRISPR repeat sequence and the minimum tracrRNA sequence can bind to the site-directed polypeptide. At least a part of the minimum CRISPR repeat sequence can hybridize to the minimum tracrRNA sequence. At least a part of the minimum CRISPR repeat sequence can comprise at least about 30%, about 40%, about 50%, about 60%, about 65%, about 70%, about 75%, about 80%, about 85%, about 90%, about 95%, or 100% complementary to the minimum tracrRNA sequence. At least a part of the minimum CRISPR repeat sequence can comprise at most about 30%, about 40%, about 50%, about 60%, about 65%, about 70%, about 75%, about 80%, about 85%, about 90%, about 95%, or 100% complementary to the minimum tracrRNA sequence.

The minimum CRISPR repeat sequence can have a length from about 7 nucleotides to about 100 nucleotides. For example, the length of the minimum CRISPR repeat sequence is from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. The minimum CRISPR repeat sequence can be approximately 9 nucleotides in length. The minimum CRISPR repeat sequence can be approximately 12 nucleotides in length.

The minimum CRISPR repeat sequence can be at least about 60% identical to a reference minimum CRISPR repeat sequence (e.g., wild-type crRNA from S. pyogenes or S. aureus) over a stretch of at least 6, 7, or 8 contiguous nucleotides. For example, the minimum CRISPR repeat sequence can be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

A minimum tracrRNA sequence can be a sequence with at least about 30%, about 40%, about 50%, about 60%, about 65%, about 70%, about 75%, about 80%, about 85%, about 90%, about 95%, or 100% sequence identity to a reference tracrRNA sequence (e.g., wild type tracrRNA from S. pyogenes or S. aureus).

A minimum tracrRNA sequence can comprise nucleotides that hybridize to a minimum CRISPR repeat sequence in a cell. A minimum tracrRNA sequence and a minimum CRISPR repeat sequence form a duplex, i.e. a base-paired double-stranded structure. Together, the minimum tracrRNA sequence and the minimum CRISPR repeat bind to a site-directed polypeptide. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA from S. pyogenes can consist of tracrRNA nt 23-48 described in Jinek et al., supra.

The minimum tracrRNA sequence can be at least about 60% identical to a reference minimum tracrRNA (e.g., wild type, tracrRNA from S. pyogenes or S. aureus) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides.

The duplex can comprise a mismatch (i.e., the two strands of the duplex are not 100% complementary). The duplex can comprise at least about 1, 2, 3, 4, or 5 or mismatches. In some examples, the duplex comprises at most about 1, 2, 3, 4, or 5 or mismatches. The duplex can comprise no more than 2 mismatches.

### Bulges

In some cases, there can be a “bulge” in the duplex between the minimum CRISPR RNA and the minimum tracrRNA. A bulge is an unpaired region of nucleotides within the duplex. A bulge can contribute to the binding of the duplex to the site-directed polypeptide. The number of unpaired nucleotides on the two sides of the duplex can be different.

In one example, a bulge can be modelled on tracrRNA sequence strand. In other examples, bulges or the unpaired nucleotides can be on the crRNA. Other examples can include multiple bulges on one or more strands. These may occur with or without unpaired nucleotides or changes in the sequence.

A bulge on the minimum CRISPR repeat side of the duplex can comprise at least 1, 2, 3, 4, or 5 or more unpaired nucleotides. The number of bulges in the minimum crRNA sequence side of the duplex can be 1, 2, 3, 4, 5 or more.

A bulge on the minimum tracrRNA sequence side of the duplex can comprise at least 1, 2, 3, 4, 5, 6, 7, 8, 9, or 10 or more unpaired nucleotides. The number of bulges in the minimum tracrRNA sequence side of the duplex can be 1, 2, 3, 4, 5 or more.

A bulge can include wobble pairing or nucleotides not thought to bind.

The sequence of the crRNA and tracrRNA sequence can be modified to have base swaps or have additions or deletions. These changes can be introduced with and without added bulges.

### Hairpins

In various examples, one or more hairpins can be located 3′ to the minimum tracrRNA in the 3′ tracrRNA sequence.

The hairpin can start at least about 1, 2, 3, 4, 5, 6, 7, 8, 9, 10, 15, or 20 or more nucleotides 3′ from the last paired nucleotide in the minimum CRISPR repeat and minimum tracrRNA sequence duplex. The hairpin can start at most about 1, 2, 3, 4, 5, 6, 7, 8, 9 or 10 or more nucleotides 3′ of the last paired nucleotide in the minimum CRISPR repeat and minimum tracrRNA sequence duplex.

The hairpin can comprise at least about 1, 2, 3, 4, 5, 6, 7, 8, 9, 10, 15, or 20 or more consecutive nucleotides. The hairpin can comprise at most about 1, 2, 3, 4, 5, 6, 7, 8, 9, 10, 15, or more consecutive nucleotides.

The hairpin can comprise a CC dinucleotide (i.e., two consecutive cytosine nucleotides).

The hairpin can comprise duplexed nucleotides (e.g., nucleotides in a hairpin, hybridized together). For example, a hairpin can comprise a CC dinucleotide that is hybridized to a GG dinucleotide in a hairpin duplex of the 3′ tracrRNA sequence.

One or more of the hairpins can interact with guide RNA-interacting regions of a site-directed polypeptide.

In some examples, there are two or more hairpins, and in some other examples there are three or more hairpins.

3′ tracrRNA Sequence

A 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes or S. aureus).

The 3′ tracrRNA sequence can have a length from about 6 nucleotides to about 100 nucleotides. For example, the 3′ tracrRNA sequence can have a length from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. The 3′ tracrRNA sequence can have a length of approximately 14 nucleotides.

The 3′ tracrRNA sequence can be at least about 60% identical to a reference 3′ tracrRNA sequence (e.g., wild type 3′ tracrRNA sequence from S. pyogenes or S. aureus) over a stretch of at least 6, 7, or 8 contiguous nucleotides. For example, the 3′ tracrRNA sequence can be at least about 60% identical, about 65% identical, about 70% identical, about 75% identical, about 80% identical, about 85% identical, about 90% identical, about 95% identical, about 98% identical, about 99% identical, or 100% identical, to a reference 3′ tracrRNA sequence (e.g., wild type 3′ tracrRNA sequence from S. pyogenes or S. aureus) over a stretch of at least 6, 7, or 8 contiguous nucleotides.

The 3′ tracrRNA sequence can comprise more than one duplexed region (e.g., hairpin, hybridized region). The 3′ tracrRNA sequence can comprise two duplexed regions.

The 3′ tracrRNA sequence can comprise a stem loop structure. The stem loop structure in the 3′ tracrRNA can comprise at least 1, 2, 3, 4, 5, 6, 7, 8, 9, 10, 15 or 20 or more nucleotides. The stem loop structure in the 3′ tracrRNA can comprise at most 1, 2, 3, 4, 5, 6, 7, 8, 9 or 10 or more nucleotides. The stem loop structure can comprise a functional moiety. For example, the stem loop structure can comprise an aptamer, a ribozyme, a protein-interacting hairpin, a CRISPR array, an intron, or an exon. The stem loop structure can comprise at least about 1, 2, 3, 4, or 5 or more functional moieties. The stem loop structure can comprise at most about 1, 2, 3, 4, or 5 or more functional moieties.

The hairpin in the 3′ tracrRNA sequence can comprise a P-domain. The P-domain can comprise a double-stranded region in the hairpin.

tracrRNA Extension Sequence

A tracrRNA extension sequence can be provided whether the tracrRNA is in the context of single-molecule guides or double-molecule guides. The tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, or 400 nucleotides. The tracrRNA extension sequence can have a length from about 20 to about 5000 or more nucleotides. The tracrRNA extension sequence can have a length of more than 1000 nucleotides. The tracrRNA extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. The tracrRNA extension sequence can have a length of less than 1000 nucleotides. The tracrRNA extension sequence can comprise less than 10 nucleotides in length. The tracrRNA extension sequence can be 10-30 nucleotides in length. The tracrRNA extension sequence can be 30-70 nucleotides in length.

The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt. The functional moiety can function in a eukaryotic cell. The functional moiety can function in a prokaryotic cell. The functional moiety can function in both eukaryotic and prokaryotic cells.

Non-limiting examples of suitable tracrRNA extension functional moieties include a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like). The tracrRNA extension sequence can comprise a primer binding site or a molecular index (e.g., barcode sequence). The tracrRNA extension sequence can comprise one or more affinity tags.

### Single-Molecule Guide Linker Sequence

The linker sequence of a single-molecule guide nucleic acid can have a length from about 3 nucleotides to about 100 nucleotides. In Jinek et al., supra, for example, a simple 4 nucleotide “tetraloop” (-GAAA-) was used, Science, 337(6096):816-821 (2012). An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker will not comprise sequences that have extensive regions of homology with other portions of the guide RNA, which might cause intramolecular binding that could interfere with other functional regions of the guide. In Jinek et al., supra, a simple 4 nucleotide sequence -GAAA- was used, Science, 337(6096):816-821 (2012), but numerous other sequences, including longer sequences can likewise be used.

The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. In some examples, the linker sequence can comprise at most about 1, 2, 3, 4, or 5 or more functional moieties.

### Target Sequence Selection

Shifts in the location of the 5′ boundary and/or the 3′ boundary relative to particular reference loci can be used to facilitate or enhance particular applications of gene editing, which depend in part on the endonuclease system selected for the editing, as further described and illustrated herein.

In a first nonlimiting example of such target sequence selection, many endonuclease systems have rules or criteria that can guide the initial selection of potential target sites for cleavage, such as the requirement of a PAM sequence motif in a particular position adjacent to the DNA cleavage sites in the case of CRISPR Type II or Type V endonucleases.

In another nonlimiting example of target sequence selection or optimization, the frequency of off-target activity for a particular combination of target sequence and gene editing endonuclease (i.e. the frequency of DSBs occurring at sites other than the selected target sequence) can be assessed relative to the frequency of on-target activity. In some cases, cells that have been correctly edited at the desired locus can have a selective advantage relative to other cells. Illustrative, but nonlimiting, examples of a selective advantage include the acquisition of attributes such as enhanced rates of replication, persistence, resistance to certain conditions, enhanced rates of successful engraftment or persistence in vivo following introduction into a patient, and other attributes associated with the maintenance or increased numbers or viability of such cells. In other cases, cells that have been correctly edited at the desired locus can be positively selected for by one or more screening methods used to identify, sort or otherwise select for cells that have been correctly edited. Both selective advantage and directed selection methods can take advantage of the phenotype associated with the correction. In some cases, cells can be edited two or more times in order to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

Whether any selective advantage is applicable or any directed selection is to be applied in a particular case, target sequence selection can also be guided by consideration of off-target frequencies in order to enhance the effectiveness of the application and/or reduce the potential for undesired alterations at sites other than the desired target. As described further and illustrated herein and in the art, the occurrence of off-target activity can be influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs can be regularly induced and repaired in normal cells. During repair, the original sequence can be reconstructed with complete fidelity, however, in some cases, small insertions or deletions (referred to as “indels”) are introduced at the DSB site.

DSBs can also be specifically induced at particular locations, as in the case of the endonuclease systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can comprise as few as ten base pairs or less, can also be used to bring about desired deletions. For example, a single DSB can be introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which can or cannot be desired given the particular circumstances.

### Nucleic Acid Modifications

In some cases, polynucleotides introduced into cells can comprise one or more modifications that can be used individually or in combination, for example, to enhance activity, stability or specificity, alter delivery, reduce innate immune responses in host cells, or for other enhancements, as further described herein and known in the art.

In certain examples, modified polynucleotides can be used in the CRISPR/Cas9/Cpf1 system, in which case the guide RNAs (either single-molecule guides or double-molecule guides) and/or a DNA or an RNA encoding a Cas or Cpf1 endonuclease introduced into a cell can be modified, as described and illustrated below. Such modified polynucleotides can be used in the CRISPR/Cas9/Cpf1 system to edit any one or more genomic loci.

Using the CRISPR/Cas9/Cpf1 system for purposes of nonlimiting illustrations of such uses, modifications of guide RNAs can be used to enhance the formation or stability of the CRISPR/Cas9/Cpf1 genome editing complex comprising guide RNAs, which can be single-molecule guides or double-molecule, and a Cas or Cpf1 endonuclease. Modifications of guide RNAs can also or alternatively be used to enhance the initiation, stability or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also, or alternatively, be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in aspects in which a Cas or Cpf1 endonuclease is introduced into the cell to be edited via an RNA that needs to be translated in order to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (i.e. the endonuclease), and/or modifications that decrease the likelihood or degree to which the RNAs introduced into cells elicit innate immune responses.

Combinations of modifications, such as the foregoing and others, can likewise be used. In the case of CRISPR/Cas9/Cpf1, for example, one or more types of modifications can be made to guide RNAs (including those exemplified above), and/or one or more types of modifications can be made to RNAs encoding Cas or Cpf1 endonuclease (including those exemplified above).

By way of illustration, guide RNAs used in the CRISPR/Cas9/Cpf1 system, or other smaller RNAs can be readily synthesized by chemical means, enabling a number of modifications to be readily incorporated, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach that can be used for generating chemically-modified RNAs of greater length is to produce two or more molecules that are ligated together. Much longer RNAs, such as those encoding a Cas9 or Cpf1 endonuclease, are more readily generated enzymatically. While fewer types of modifications are available for use in enzymatically produced RNAs, there are still modifications that can be used to, e.g., enhance stability, reduce the likelihood or degree of innate immune response, and/or enhance other attributes, as described further below and in the art; and new types of modifications are regularly being developed.

By way of illustration of various types of modifications, especially those used frequently with smaller chemically synthesized RNAs, modifications can comprise one or more nucleotides modified at the 2′ position of the sugar, in some aspects, a 2′-O-alkyl, 2′-O-alkyl-O-alkyl, or 2′-fluoro-modified nucleotide. In some examples, RNA modifications can comprise 2′-fluoro, 2′-amino or 2′ O-methyl modifications on the ribose of pyrimidines, abasic residues, or an inverted base at the 3′ end of the RNA. Such modifications can be routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH,˜N(CH₃)˜O˜CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones [see De Mesmaeker et al., Ace. Chem. Res., 28:366-374 (1995)]; morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, Biochemistry, 41(14): 4503-4510 (2002); Genesis, Volume 30, Issue 3, (2001); Heasman, Dev. Biol., 243: 209-214 (2002); Nasevicius et al., Nat. Genet., 26:216-220 (2000); Lacerra et al., Proc. Natl. Acad. Sci., 97: 9591-9596 (2000); and U.S. Pat. No. 5,034,506, issued Jul. 23, 1991.

Cyclohexenyl nucleic acid oligonucleotide mimetics are described in Wang et al., J. Am. Chem. Soc., 122: 8595-8602 (2000).

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

One or more substituted sugar moieties can also be included, e.g., one of the following at the 2′ position: OH, SH, SCH₃, F, OCN, OCH₃, OCH₃ O(CH₂)n CH₃, O(CH₂)n NH₂, or O(CH₂)n CH₃, where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some aspects, a modification includes 2′-methoxyethoxy (2′-O—CH₂CH₂OCH₃, also known as 2′-O-(2-methoxyethyl)) (Martin et al, HeIv. Chim. Acta, 1995, 78, 486). Other modifications include 2′-methoxy (2′-O—CH₃), 2′-propoxy (2′-OCH₂ CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some examples, both a sugar and an internucleoside linkage, i.e., the backbone, of the nucleotide units can be replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide can be replaced with an amide containing backbone, for example, an aminoethylglycine backbone. The nucleobases can be retained and bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262. Further teaching of PNA compounds can be found in Nielsen et al, Science, 254: 1497-1500 (1991).

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Kornberg, A., DNA Replication, W. H. Freeman & Co., San Francisco, pp 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases can comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition', 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, ‘Antisense Research and Applications’, CRC Press, Boca Raton, 1993, pp. 276-278) and are aspects of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and US Patent Application Publication 2003/0158403.

Thus, the term “modified” refers to a non-natural sugar, phosphate, or base that is incorporated into a guide RNA, an endonuclease, or both a guide RNA and an endonuclease. It is not necessary for all positions in a given oligonucleotide to be uniformly modified, and in fact more than one of the aforementioned modifications can be incorporated in a single oligonucleotide, or even in a single nucleoside within an oligonucleotide.

The guide RNAs and/or mRNA (or DNA) encoding an endonuclease (or DNA encoding an endonuclease) can be chemically linked to one or more moieties or conjugates that enhance the activity, cellular distribution, or cellular uptake of the oligonucleotide. Such moieties comprise, but are not limited to, lipid moieties such as a cholesterol moiety [Letsinger et al., Proc. Natl. Acad. Sci. USA, 86: 6553-6556 (1989)]; cholic acid [Manoharan et al., Bioorg. Med. Chem. Let., 4: 1053-1060 (1994)]; a thioether, e.g., hexyl-S-tritylthiol [Manoharan et al, Ann. N. Y. Acad. Sci., 660: 306-309 (1992) and Manoharan et al., Bioorg. Med. Chem. Let., 3: 2765-2770 (1993)]; a thiocholesterol [Oberhauser et al., Nucl. Acids Res., 20: 533-538 (1992)]; an aliphatic chain, e.g., dodecandiol or undecyl residues [Kabanov et al., FEBS Lett., 259: 327-330 (1990) and Svinarchuk et al., Biochimie, 75: 49-54 (1993)]; a phospholipid, e.g., di-hexadecyl-rac-glycerol or triethylammonium 1,2-di-O-hexadecyl-rac-glycero-3-H-phosphonate [Manoharan et al., Tetrahedron Lett., 36: 3651-3654 (1995) and Shea et al., Nucl. Acids Res., 18: 3777-3783 (1990)]; a polyamine or a polyethylene glycol chain [Mancharan et al., Nucleosides & Nucleotides, 14: 969-973 (1995)]; adamantane acetic acid [Manoharan et al., Tetrahedron Lett., 36: 3651-3654 (1995)]; a palmityl moiety [(Mishra et al., Biochim. Biophys. Acta, 1264: 229-237 (1995)]; or an octadecylamine or hexylamino-carbonyl-t oxycholesterol moiety [Crooke et al., J. Pharmacol. Exp. Ther., 277: 923-937 (1996)]. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., Protein Pept Lett. 21(10):1025-30 (2014). Other systems known in the art and regularly developed can be used to target biomolecules of use in the present case and/or complexes thereof to particular target cells of interest.

These targeting moieties or conjugates can include conjugate groups covalently bound to functional groups, such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this disclosure, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Longer polynucleotides that are less amenable to chemical synthesis and are typically produced by enzymatic synthesis can also be modified by various means. Such modifications can include, for example, the introduction of certain nucleotide analogs, the incorporation of particular sequences or other moieties at the 5′ or 3′ ends of molecules, and other modifications. By way of illustration, the mRNA encoding Cas9 is approximately 4 kb in length and can be synthesized by in vitro transcription. Modifications to the mRNA can be applied to, e.g., increase its translation or stability (such as by increasing its resistance to degradation with a cell), or to reduce the tendency of the RNA to elicit an innate immune response that is often observed in cells following introduction of exogenous RNAs, particularly longer RNAs such as that encoding Cas9.

Numerous such modifications have been described in the art, such as polyA tails, 5′ cap analogs (e.g., Anti Reverse Cap Analog (ARCA) or m7G(5′)ppp(5′)G (mCAP)), modified 5′ or 3′ untranslated regions (UTRs), use of modified bases (such as Pseudo-UTP, 2-Thio-UTP, 5-Methylcytidine-5′-Triphosphate (5-Methyl-CTP) or N6-Methyl-ATP), or treatment with phosphatase to remove 5′ terminal phosphates. These and other modifications are known in the art, and new modifications of RNAs are regularly being developed.

There are numerous commercial suppliers of modified RNAs, including for example, TriLink Biotech, AxoLabs, Bio-Synthesis Inc., Dharmacon and many others. As described by TriLink, for example, 5-Methyl-CTP can be used to impart desirable characteristics, such as increased nuclease stability, increased translation or reduced interaction of innate immune receptors with in vitro transcribed RNA. 5-Methylcytidine-5′-Triphosphate (5-Methyl-CTP), N6-Methyl-ATP, as well as Pseudo-UTP and 2-Thio-UTP, have also been shown to reduce innate immune stimulation in culture and in vivo while enhancing translation, as illustrated in publications by Kormann et al. and Warren et al. referred to below.

It has been shown that chemically modified mRNA delivered in vivo can be used to achieve improved therapeutic effects; see, e.g., Kormann et al., Nature Biotechnology 29, 154-157 (2011). Such modifications can be used, for example, to increase the stability of the RNA molecule and/or reduce its immunogenicity. Using chemical modifications such as Pseudo-U, N6-Methyl-A, 2-Thio-U and 5-Methyl-C, it was found that substituting just one quarter of the uridine and cytidine residues with 2-Thio-U and 5-Methyl-C respectively resulted in a significant decrease in toll-like receptor (TLR) mediated recognition of the mRNA in mice. By reducing the activation of the innate immune system, these modifications can be used to effectively increase the stability and longevity of the mRNA in vivo; see, e.g., Kormann et al., supra.

It has also been shown that repeated administration of synthetic messenger RNAs incorporating modifications designed to bypass innate anti-viral responses can reprogram differentiated human cells to pluripotency. See, e.g., Warren, et al., Cell Stem Cell, 7(5):618-30 (2010). Such modified mRNAs that act as primary reprogramming proteins can be an efficient means of reprogramming multiple human cell types. Such cells are referred to as induced pluripotency stem cells (iPSCs), and it was found that enzymatically synthesized RNA incorporating 5-Methyl-CTP, Pseudo-UTP and an Anti Reverse Cap Analog (ARCA) could be used to effectively evade the cell's antiviral response; see, e.g., Warren et al., supra.

Other modifications of polynucleotides described in the art include, for example, the use of polyA tails, the addition of 5′ cap analogs (such as m7G(5′)ppp(5′)G (mCAP)), modifications of 5′ or 3′ untranslated regions (UTRs), or treatment with phosphatase to remove 5′ terminal phosphates—and new approaches are regularly being developed.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., Annual Review of Chemical and Biomolecular Engineering, 2: 77-96 (2011); Gaglione and Messere, Mini Rev Med Chem, 10(7):578-95 (2010); Chernolovskaya et al, Curr Opin Mol Ther., 12(2):158-67 (2010); Deleavey et al., Curr Protoc Nucleic Acid Chem Chapter 16:Unit 16.3 (2009); Behlke, Oligonucleotides 18(4):305-19 (2008); Fucini et al., Nucleic Acid Ther 22(3): 205-210 (2012); Bremsen et al., Front Genet 3:154 (2012).

As noted above, there are a number of commercial suppliers of modified RNAs, many of which have specialized in modifications designed to improve the effectiveness of siRNAs. A variety of approaches are offered based on various findings reported in the literature. For example, Dharmacon notes that replacement of a non-bridging oxygen with sulfur (phosphorothioate, PS) has been extensively used to improve nuclease resistance of siRNAs, as reported by Kole, Nature Reviews Drug Discovery 11:125-140 (2012). Modifications of the 2′-position of the ribose have been reported to improve nuclease resistance of the internucleotide phosphate bond while increasing duplex stability (Tm), which has also been shown to provide protection from immune activation. A combination of moderate PS backbone modifications with small, well-tolerated 2′-substitutions (2′-O-Methyl, 2′-Fluoro, 2′-Hydro) have been associated with highly stable siRNAs for applications in vivo, as reported by Soutschek et al. Nature 432:173-178 (2004); and 2′-O-Methyl modifications have been reported to be effective in improving stability as reported by Volkov, Oligonucleotides 19:191-202 (2009). With respect to decreasing the induction of innate immune responses, modifying specific sequences with 2′-O-Methyl, 2′-Fluoro, 2′-Hydro have been reported to reduce TLR7/TLR8 interaction while generally preserving silencing activity; see, e.g., Judge et al., Mol. Ther. 13:494-505 (2006); and Cekaite et al., J. Mol. Biol. 365:90-108 (2007). Additional modifications, such as 2-thiouracil, pseudouracil, 5-methylcytosine, 5-methyluracil, and N6-methyladenosine have also been shown to minimize the immune effects mediated by TLR3, TLR7, and TLR8; see, e.g., Kariko, K. et al., Immunity 23:165-175 (2005).

As is also known in the art, and commercially available, a number of conjugates can be applied to polynucleotides, such as RNAs, for use herein that can enhance their delivery and/or uptake by cells, including for example, cholesterol, tocopherol and folic acid, lipids, peptides, polymers, linkers and aptamers; see, e.g., the review by Winkler, Ther. Deliv. 4:791-809 (2013), and references cited therein.

### Codon-Optimization

A polynucleotide encoding a site-directed polypeptide can be codon-optimized according to methods standard in the art for expression in the cell containing the target DNA of interest. For example, if the intended target nucleic acid is in a human cell, a human codon-optimized polynucleotide encoding Cas9 is contemplated for use for producing the Cas9 polypeptide.

### Complexes of a DNA-Targeting Nucleic Acid and a Site-Directed Polypeptide

A DNA-targeting nucleic acid interacts with a site-directed polypeptide (e.g., a nucleic acid-guided nuclease such as Cas9), thereby forming a complex. The DNA-targeting nucleic acid guides the site-directed polypeptide to a target nucleic acid.

### Ribonucleoprotein Complexes (RNPs)

The site-directed polypeptide and DNA-targeting nucleic acid can each be administered separately to a cell or a patient. On the other hand, the site-directed polypeptide can be pre-complexed with one or more guide RNAs (e.g.: one or more sgRNA), or one or more crRNA together with a tracrRNA. The pre-complexed material can then be administered to a cell or a patient. Such pre-complexed material is known as a RNP. The site-directed polypeptide in the RNP can be, for example, a Cas9 endonuclease or a Cpf1 endonuclease. The site-directed polypeptide can be flanked at the N-terminus, the C-terminus, or both the N-terminus and C-terminus by one or more nuclear localization signals (NLSs). For example, a Cas9 endonuclease can be flanked by two NLSs, one NLS located at the N-terminus and the second NLS located at the C-terminus. The NLS can be any NLS known in the art, such as a SV40 NLS. The weight ratio of DNA-targeting nucleic acid to site-directed polypeptide in the RNP can be 1:1. For example, the weight ratio of sgRNA to Cas9 endonuclease in the RNP can be 1:1.

### Nucleic Acids Encoding System Components

The present disclosure provides a nucleic acid comprising a nucleotide sequence encoding a DNA-targeting nucleic acid of the disclosure, a site-directed polypeptide of the disclosure, and/or any nucleic acid or proteinaceous molecule necessary to carry out the aspects of the methods of the disclosure.

The nucleic acid encoding a DNA-targeting nucleic acid of the disclosure, a site-directed polypeptide of the disclosure, and/or any nucleic acid or proteinaceous molecule necessary to carry out the aspects of the methods of the disclosure can comprise a vector (e.g., a recombinant expression vector).

The term “vector” refers to a nucleic acid molecule capable of transporting another nucleic acid to which it has been linked. A vector can be an expression vector. An “expression vector” is a replicon, such as plasmid, phage, virus, or cosmid, to which another DNA segment, i.e. an “insert”, can be attached so as to bring about the replication of the attached segment in a cell.

One type of vector is a “plasmid”, which refers to a circular double-stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operatively linked. Such vectors are referred to herein as “recombinant expression vectors”, or “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Expression vectors contemplated include, but are not limited to, viral vectors (e.g. based on vaccinia virus; poliovirus; adenovirus; adeno-associated virus; SV40; herpes simplex virus; human immunodeficiency virus; a retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and other recombinant vectors.

Other vectors contemplated for eukaryotic target cells include, but are not limited to, the vectors: pXT1, pSG5, pSVK3, pBPV, pMSG, and pSVLSV40 (Pharmacia). Other vectors can be used as long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

In some examples, a nucleic acid encoding a DNA-targeting nucleic acid of the disclosure, a site-directed polypeptide of the disclosure, and/or any nucleic acid or proteinaceous molecule necessary to carry out the aspects of the disclosure is operably linked to a control element, e.g., a transcriptional control element, such as a promoter. The transcriptional control element can be functional in either a eukaryotic cell, e.g., a mammalian cell; or a prokaryotic cell (e.g., bacterial or archaeal cell). In some examples, a nucleotide sequence encoding a guide RNA and/or a site-directed modifying polypeptide can be operably linked to multiple control elements that allow expression of the nucleotide sequence encoding a guide RNA and/or a site-directed modifying polypeptide in both prokaryotic and eukaryotic cells.

A promoter can be a constitutively active promoter (i.e., a promoter that is constitutively in an active/“ON” state), it can be an inducible promoter (i.e., a promoter whose state, active/“ON” or inactive/“OFF”, is controlled by an external stimulus, e.g., the presence of a particular temperature, compound, or protein.), it can be a spatially restricted promoter (i.e., transcriptional control element, enhancer, etc.) (e.g., tissue specific promoter, cell type specific promoter, etc.), and it can be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas endonuclease, various promoters such as RNA polymerase III promoters, including for example U6 and H1, can be advantageous. Descriptions of and parameters for enhancing the use of such promoters are known in art, and additional information and approaches are regularly being described; see, e.g., Ma, H. et al., Molecular Therapy—Nucleic Acids 3, e161 (2014) doi:10.1038/mtna.2014.12.

The expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.). The non-native tags can be fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter, etc.).

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

Spatially restricted promoters can also be referred to as enhancers, transcriptional control elements, control sequences, etc. Any convenient spatially restricted promoter can be used and the choice of suitable promoter (e.g., a liver-specific promoter, a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, liver-specific promoters, neuron-specific promoters, adipocyte-specific promoters, cardiomyocyte-specific promoters, smooth muscle-specific promoters, photoreceptor-specific promoters, etc.

Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca²⁺-calmodulin-dependent protein kinase II-alpha (CamKIIa) promoter (see, e.g., Mayford et al. (1996) Proc. Natl. Acad. Sci. USA 93:13250; and Casanova et al. (2001) Genesis 31:37); a CMV enhancer/platelet-derived growth factor-0 promoter (see, e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (see, e.g., Akyilrek et al. (2000) Mol. Med. 6:983; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

To modulate kinetics of self-inactivation and on-target activities, a weaker promoter driving gRNA(s) for self-inactivation and a stronger promoter to drive expression of gRNA for on-target activity can also be used.

Methods of introducing a nucleic acid into a host cell are known in the art, and any known method can be used to introduce a nucleic acid (e.g., an expression construct) into a cell. Nucleotides encoding a guide RNA (introduced either as DNA or RNA) and/or a site-directed modifying polypeptide (introduced as DNA or RNA) and/or a donor polynucleotide can be provided to the cells using well-developed transfection techniques; see, e.g. Angel and Yanik (2010) PLoS ONE 5(7): e 11756, and the commercially available TransMessenger® reagents from Qiagen, StemfectTM RNA Transfection Kit from Stemgent, and TranslT®-mRNA Transfection Kit from Mims Bio LLC (See, also Beumer et al. (2008) Efficient gene targeting in Drosophila by direct embryo injection with zinc-finger nucleases. PNAS 105(50):19821-19826). Alternatively, nucleic acids encoding a guide RNA and/or a site-directed modifying polypeptide and/or a chimeric site-directed modifying polypeptide and/or a donor polynucleotide can be provided on DNA vectors. Many vectors, e.g. plasmids, cosmids, minicircles, phage, viruses, etc., useful for transferring nucleic acids into target cells are available. The vectors comprising the nucleic acid(s) can be maintained episomally, e.g. as plasmids, minicircle DNAs, viruses such cytomegalovirus, adenovirus, etc., or they can be integrated into the target cell genome, through homologous recombination or random integration, e.g. retrovirus-derived vectors such as MMLV, HIV-1, ALV, etc.

Vectors can be provided directly to the cells. In other words, the cells are contacted with vectors comprising the nucleic acid encoding guide RNA and/or a site-directed modifying polypeptide and/or a chimeric site-directed modifying polypeptide and/or a donor polynucleotide such that the vectors are taken up by the cells. Methods for contacting cells with nucleic acid vectors that are plasmids, including electroporation, calcium chloride transfection, microinjection, and lipofection are well known in the art. For viral vector delivery, the cells can be contacted with viral particles comprising the nucleic acid encoding a guide RNA and/or a site-directed modifying polypeptide and/or a chimeric site-directed modifying polypeptide and/or a donor polynucleotide. Retroviruses, for example, lentiviruses, are suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid can be packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line can be used to ensure that the cells are targeted by the packaged viral particles. Methods of introducing the retroviral vectors comprising the nucleic acid encoding the reprogramming factors into packaging cell lines and of collecting the viral particles that are generated by the packaging lines are well known in the art. Nucleic acids can also be introduced by direct micro-injection (e.g., injection of RNA into a zebrafish embryo).

Vectors used for providing the nucleic acids encoding guide RNA and/or a site-directed modifying polypeptide and/or a chimeric site-directed modifying polypeptide and/or a donor polynucleotide to the cells can typically comprise suitable promoters for driving the expression, that is, transcriptional activation, of the nucleic acid of interest. In other words, the nucleic acid of interest will be operably linked to a promoter. This can include ubiquitously acting promoters, for example, the CMV-13-actin promoter, or inducible promoters, such as promoters that are active in particular cell populations or that respond to the presence of drugs such as tetracycline. By transcriptional activation, it can be intended that transcription will be increased above basal levels in the target cell by at least about 10 fold, by at least about 100 fold, more usually by at least about 1000 fold. In addition, vectors used for providing a guide RNA and/or a site-directed modifying polypeptide and/or a chimeric site-directed modifying polypeptide and/or a donor polynucleotide to the cells can include nucleic acid sequences that encode for selectable markers in the target cells, so as to identify cells that have taken up the guide RNA and/or a site-directed modifying polypeptide and/or a chimeric site-directed modifying polypeptide and/or a donor polynucleotide.

The nucleic acid encoding a DNA-targeting nucleic acid of the disclosure and/or a site-directed polypeptide can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

### Delivery

The delivery systems can be viral vectors, lipid nonaparticles (LNPs) or synthetic polymers. Timing of delivery of AAV vectors and LNPs can be varied (delivered at the same time or sequentially) to further achive spatiotemporal control of Cas9 expression and the self-inactivation.

Guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In further alternative aspects, the DNA endonuclease can be delivered as one or more polypeptides, either alone or pre-complexed with one or more guide RNAs, or one or more crRNA together with a tracrRNA.

Polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other polynucleotides).

Polynucleotides, such as guide RNA, sgRNA, and mRNA or DNA encoding an endonuclease, can be delivered to a cell or a patient by a lipid nanoparticle (LNP).

A LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses.

LNPs can also be comprised of hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N₁₂-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

The lipids can be combined in any number of molar ratios to produce a LNP. In addition, the polynucleotide(s) can be combined with lipid(s) in a wide range of molar ratios to produce a LNP.

As stated previously, the site-directed polypeptide and DNA-targeting nucleic acid can each be administered separately to a cell or a patient. On the other hand, the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

RNA is capable of forming specific interactions with RNA or DNA. While this property is exploited in many biological processes, it also comes with the risk of promiscuous interactions in a nucleic acid-rich cellular environment. One solution to this problem is the formation of ribonucleoprotein particles (RNPs), in which the RNA is pre-complexed with an endonuclease. Another benefit of the RNP is protection of the RNA from degradation.

The endonuclease in the RNP can be modified or unmodified. Likewise, the gRNA, crRNA, tracrRNA, or sgRNA can be modified or unmodified. Numerous modifications are known in the art and can be used.

The endonuclease and sgRNA can be generally combined in a 1:1 molar ratio. Alternatively, the endonuclease, crRNA and tracrRNA can be generally combined in a 1:1:1 molar ratio. However, a wide range of molar ratios can be used to produce a RNP.

A recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692. See Table 2

TABLE 2

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | A Y631965.1 |
| AAV-11 | AY631966.1 |
| AAV-12 | DQ813647.1 |
| AAV-13 | EU285562.1 |

A method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others. See Table 3

TABLE 3

| Tissue/Cell Type | Serotype |
|---|---|
| Liver | AAV3, AAV5, AAV8, AAV9 |
| Skeletal muscle | AAV1, AAV7, AAV6, AAV8, AAV9 |
| Central nervous system | AAV5, AAV1, AAV4, AAV8, AAV9 |
| RPE | AAV5, AAV4, AAV2, AAV8, AAV9, |
| | AAVrh8R |
| Photoreceptor cells | AAV5, AAV8, AAV9, AAVrh8R |
| Lung | AAV9, AAV5 |
| Heart | AAV8 |
| Pancreas | AAV8 |
| Kidney | AAV2, AAV8 |

In addition to adeno-associated viral vectors, other viral vectors can be used. Such viral vectors include, but are not limited to, adenovirus, lentivirus, alphavirus, enterovirus, pestivirus, baculovirus, herpesvirus, Epstein Barr virus, papovavirus, poxvirus, vaccinia virus, and herpes simplex virus.

In some cases, Cas9 mRNA, sgRNA targeting one or two loci in target genes, and donor DNA are each separately formulated into lipid nanoparticles, or are all co-formulated into one lipid nanoparticle.

In some examples, Cas9 mRNA is formulated in a lipid nanoparticle, while sgRNA and donor DNA are delivered in an AAV vector.

Options are available to deliver the Cas9 nuclease as a DNA plasmid, as mRNA or as a protein. The guide RNA can be expressed from the same DNA, or can also be delivered as an RNA. The RNA can be chemically modified to alter or improve its half-life, or decrease the likelihood or degree of immune response. The endonuclease protein can be complexed with the gRNA prior to delivery. Viral vectors allow efficient delivery; split versions of Cas9 and smaller orthologs of Cas9 can be packaged in AAV, as can donors for HDR. A range of non-viral delivery methods also exist that can deliver each of these components, or non-viral and viral methods can be employed in tandem. For example, nano-particles can be used to deliver the protein and guide RNA, while AAV can be used to deliver a donor DNA.

dCas9-FokI and Other Nucleases

Combining the structural and functional properties of the nuclease platforms described above offers a further approach to genome editing that can potentially overcome some of the inherent deficiencies. As an example, the CRISPR genome editing system typically uses a single Cas9 endonuclease to create a DSB. The specificity of targeting is driven by a 20 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 catalytic function—retaining only the RNA-guided DNA binding function—and instead fusing a FokI domain to the deactivated Cas9; see, e.g., Tsai et al., Nature Biotech 32: 569-76 (2014); and Guilinger et al., Nature Biotech. 32: 577-82 (2014). Because FokI must dimerize to become catalytically active, two guide RNAs are required to tether two Cas9-FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

### Genetically Modified Cells

The term “genetically modified cell” refers to a cell that comprises at least one genetic modification introduced by genome editing (e.g., using the CRISPR/Cas9/Cpf1 system). A genetically modified cell comprising an exogenous DNA-targeting nucleic acid and/or an exogenous nucleic acid encoding a DNA-targeting nucleic acid is contemplated herein.

In some examples, a genetically modified cell can comprise any of the self-inactivating CRISPR/Cas or CRISPR/Cpf1 systems disclosed herein.

In some examples, the cell can be selected from the group consisting of: an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

The term “isolated cell” refers to a cell that has been removed from an organism in which it was originally found, or a descendant of such a cell. Optionally, the cell can be cultured in vitro, e.g., under defined conditions or in the presence of other cells. Optionally, the cell can be later introduced into a second organism or re-introduced into the organism from which it (or the cell from which it is descended) was isolated.

The term “isolated population” with respect to an isolated population of cells refers to a population of cells that has been removed and separated from a mixed or heterogeneous population of cells. In some cases, the isolated population can be a substantially pure population of cells, as compared to the heterogeneous population from which the cells were isolated or enriched. In some cases, the isolated population can be an isolated population of human progenitor cells, e.g., a substantially pure population of human progenitor cells, as compared to a heterogeneous population of cells comprising human progenitor cells and cells from which the human progenitor cells were derived.

### Host Cells

In some of the above applications, the methods can be employed to induce DNA cleavage, DNA modification, and/or transcriptional modulation in mitotic or post-mitotic cells in vivo and/or ex vivo and/or in vitro (e.g., to produce genetically modified cells that can be reintroduced into an individual). Because the guide RNA provide specificity by hybridizing to target DNA, a mitotic and/or post-mitotic cell of interest in the disclosed methods can include a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a primate, a cell from a human, etc.). Suitable host cells include naturally-occurring cells; genetically modified cells (e.g., cells genetically modified in a laboratory, e.g., by the “hand of man”); and cells manipulated in vitro in any way. In some cases, a host cell can be isolated.

Any type of cell can be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells can be from established cell lines or they can be primary cells, where “primary cells”, “primary cell lines”, and “primary cultures” are used interchangeably herein to refer to cells and cells cultures that have been derived from a and allowed to grow in vitro for a limited number of passages, i.e. splittings, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. Target cells can be in many examples unicellular organisms, or can be grown in culture.

If the cells are primary cells, such cells can be harvested from an individual by any convenient method. For example, leukocytes can be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution can be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells can be used immediately, or they can be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

Following the methods described above, a DNA region of interest can be cleaved and modified, i.e. “genetically modified”, ex vivo. In some examples, as when a selectable marker has been inserted into the DNA region of interest, the population of cells can be enriched for those comprising the genetic modification by separating the genetically modified cells from the remaining population. Prior to enriching, the “genetically modified” cells can make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells can be achieved by any convenient separation technique appropriate for the selectable marker used. For example, if a fluorescent marker has been inserted, cells can be separated by fluorescence activated cell sorting, whereas if a cell surface marker has been inserted, cells can be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells can be selected against dead cells by employing dyes associated with dead cells (e.g. propidium iodide). Any technique can be employed which is not unduly detrimental to the viability of the genetically modified cells. Cell compositions that are highly enriched for cells comprising modified DNA can be achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition can be a substantially pure composition of genetically modified cells.

Genetically modified cells produced by the methods described herein can be used immediately. Alternatively, the cells can be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells can be cultured in vitro under various culture conditions. The cells can be expanded in culture, i.e. grown under conditions that promote their proliferation. Culture medium can be liquid or semi-solid, e.g. containing agar, methylcellulose, etc. The cell population can be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture can contain growth factors to which the regulatory T cells are responsive. Growth factors, as defined herein, can be molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way can be transplanted to a subject for purposes such as gene therapy, e.g. to treat a disease or as an antiviral, antipathogenic, or anticancer therapeutic, for the production of genetically modified organisms in agriculture, or for biological research. The subject can be a neonate, a juvenile, or an adult. Of particular interest are mammalian subjects. Mammalian species that can be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) can be used for experimental investigations.

Cells can be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells can be introduced to the subject via any of the following routes: parenteral, subcutaneous, intravenous, intracranial, intraspinal, intraocular, or into spinal fluid. The cells can be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5,385,582, incorporated herein by reference); by bolus injection, e.g. by a syringe, e.g. into a joint; by continuous infusion, e.g. by cannulation, e.g. with convection (see e.g. US Application No. 20070254842, incorporated herein by reference); or by implanting a device upon which the cells have been reversably affixed (see e.g. US Application Nos. 20080081064 and 20090196903, incorporated herein by reference). Cells can also be introduced into an embryo (e.g., a blastocyst) for the purpose of generating a transgenic animal (e.g., a transgenic mouse).

The number of administrations of treatment to a subject can vary. Introducing the genetically modified cells into the subject can be a one-time event; but in certain situations, such treatment can elicit improvement for a limited period of time and require an on-going series of repeated treatments. In other situations, multiple administrations of the genetically modified cells can be required before an effect is observed. The exact protocols depend upon the disease or condition, the stage of the disease and parameters of the individual subject being treated.

### Self-Targeting/Self-Inactivating CRISPR/Cas Systems

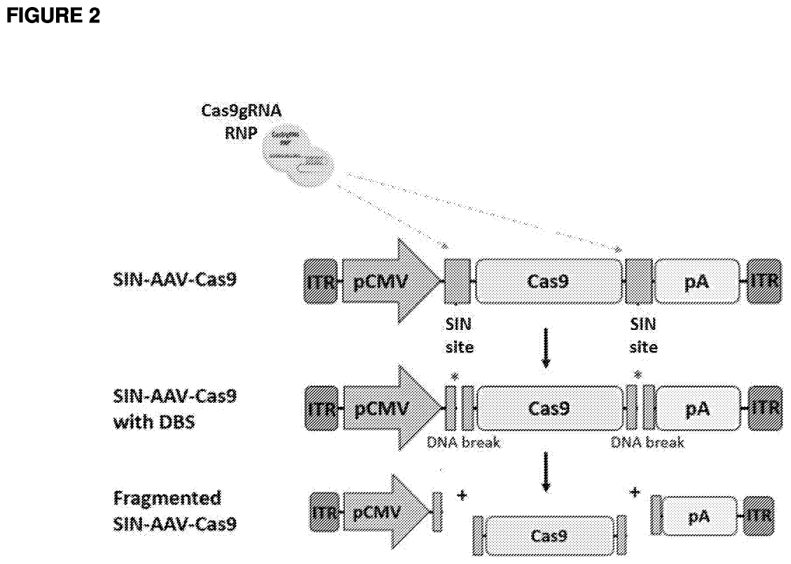

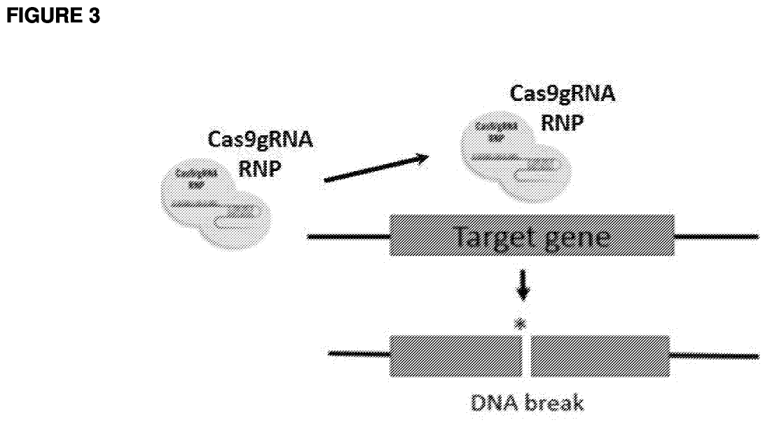

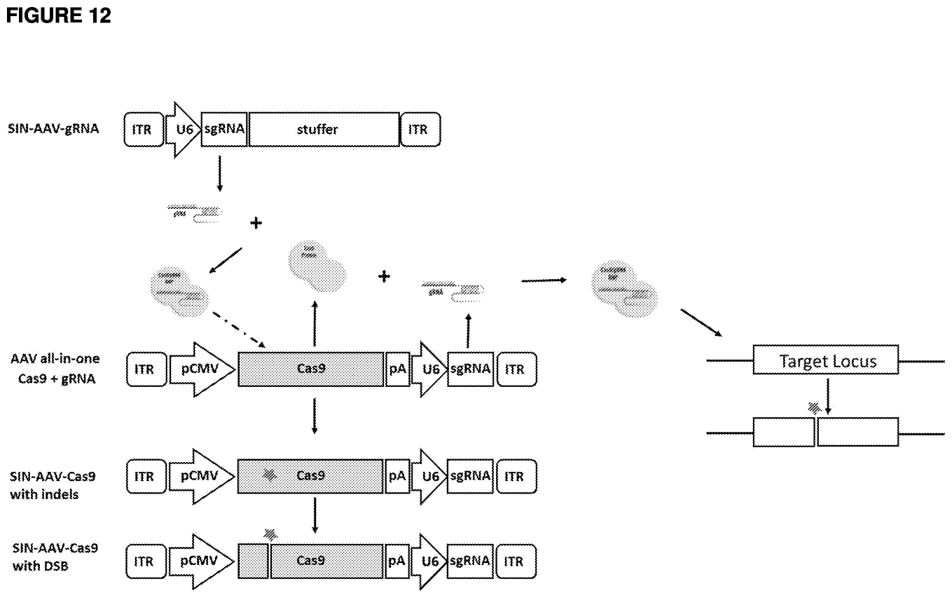

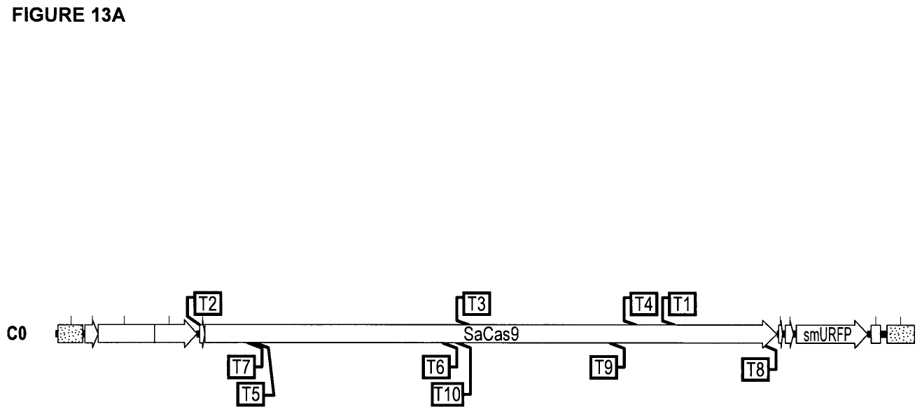

Another aspect of the disclosure is a self-targeting CRISPR/Cas or CRISPR/Cpf1 system that utilizes a non-coding targeting sequence within the CRISPR vector itself that is substantially complementary to either the site-directed polypeptide within the vector (FIG. 12), one or more non-coding sequences in the site-directed polypeptide expression vector (FIGS. 1-2), or to the target gene in the vector (FIG. 3). In some examples, the self-targeting CRISPR/Cas or CRISPR/Cpf1 system targets, but does not inactivate the system. Such self-targeting CRISPR/Cas or CRISPR/Cpf1 systems would allow for tracking of edited loci, for example.

In some examples, the self-targeting CRISPR/Cas or CRISPR/Cpf1 system can inactivate expression of the site-directed polypeptide (i.e., Cas9 or Cpf1). In this regard, after expression begins, the CRISPR system will lead to its own destruction, but before destruction is complete it will have time to edit one or more genomic copies of the target gene. The self-inactivating CRISPR/Cas or CRISPR/Cpf1 system can include SIN sites that target the coding sequence for the site-directed polypeptide itself, or that targets one or more non-coding sequences in the site-directed polypeptide expression vector (e.g., SIN sites).

In some examples, the self-targeting/self-inactivating CRISPR/Cas or CRISPR/Cpf1 system can be engineered to have altered sequences downstream of a target site to have a canonical or non-canonical PAM, such as NRG or variants thereof (e.g.: NGG, NAG or NGA). In some examples, the self-targeting/self-inactivating CRISPR/Cas or CRISPR/Cpf1 system can be engineered to have altered sequences downstream of a target site to have a canonical or non-canonical PAM, such as NNGRRN, or any variants thereof. In some examples, the self-targeting/self-inactivating CRISPR/Cas or CRISPR/Cpf1 system can be engineered to have altered sequences downstream of a target site to have a canonical or non-canonical PAM, such as NNGRRT or any variants thereof (e.g.: CTGAAT, GAGAGT, ATGAGT, CAGAGT, TTGAGT or TGGAAT).

In some examples, the self-inactivating CRISPR/Cas or CRISPR/Cpf1 system can be an “all in one” vector system. A single vector system is developmentally permissive and allows for both spatial and temporal control of the site-directed polypeptide expression in all vector transduced cells. The all-in-one system can allow for consistent delivery and expression of Cas9 or Cpf1 and gRNAs in the same cell and at a fixed ratio translating to a better editing efficiency compared to all-in-two system. In addition, presence of SIN sites within the vector can ensure transient expression of Cas9 or Cpf1, which is expected to result in better safety profile.

In some examples, the self-inactivating CRISPR/Cas or CRISPR/Cpf1 system can be an “all-in-two” vector system. The dual vector system can allow for delivery of Homology Directed Repair (HDR) templates, site-directed polypeptide, and more than one guide RNA (gRNA). Expression of more than one gRNA allows for the introduction of double-stranded breaks in the target gene and also a mutation in the coding sequence and/or a decrease or termination of Cas9 or Cpf1 expression as well as temporal control over termination of Cas9 or Cpf1 expression.

In one aspect, described herein is a self-inactivating CRISPR/Cas or CRISPR/Cpf1 system comprising a first segment comprising a nucleotide sequence that encodes a site-directed polypeptide (e.g., a CRISPR enzyme); a second segment comprising a nucleotide sequence that encodes a DNA-targeting nucleic acid (e.g., guide RNA); and one or more third segments (e.g., SIN site) comprising a nucleotide sequence that is substantially complementary to the second segment (e.g., gRNA).

In another aspect, described herein is a self-inactivating CRISPR/Cas or CRISPR/Cpf1 system comprising a first segment comprising a nucleotide sequence that encodes a site-directed polypeptide (e.g., a CRISPR enzyme); a second segment comprising a nucleotide sequence that encodes a DNA-targeting nucleic acid (e.g., gRNA or sgRNA); and one or more third segments comprising a nucleotide sequence that is substantially complementary to the nucleotide sequence of the DNA-targeting nucleic acid (e.g., SIN sites).

In another aspect, described herein is a self-inactivating CRISPR/Cas or CRISPR/Cpf1 system comprising a first segment comprising a nucleotide sequence that encodes a site-directed polypeptide (e.g., a CRISPR enzyme); a second segment comprising a nucleotide sequence that encodes a DNA-targeting nucleic acid (e.g., gRNA or sgRNA); and one or more third segments (e.g., SIN sites) comprising a nucleotide sequence that is substantially complementary to the nucleotide sequence of the DNA-targeting nucleic acid, wherein the sequence of the first segment comprises the sequence of the third segment. For example, the nucleotide sequence that encodes a site-directed polypeptide comprises a SIN site nucleotide sequence.

In some examples, the first segment comprising a nucleotide sequence that encodes a site-directed polypeptide, can further comprise a start codon, a stop codon, and a poly(A) termination site. In other examples, the first segment comprising a nucleotide sequence that encodes a site-directed polypeptide, can further comprise one or more naturally occurring or chimeric introns inserted into, upstream, and/or downstream of a Cas9 open reading frame (ORF). The chimeric intron can comprise a 5′-donor site from the first intron of the human β-globin gene and the branch and a 3′-acceptor site from the intron of an immunoglobulin gene heavy chain variable region. The chimeric intron introduced into Cas9 ORF can be used to insert one or more gRNA binding sites utilized for self-inactivation (e.g.: SIN site). Introns and/or their splicing can enhance almost every step of gene expression, from transcription to translation. For example, intron-containing transgenes in mice are transcribed up to 100-fold more efficiently than the same genes lacking introns. The enhancing effects of introns on the posttranscriptional stages of gene expression are commonly attributed to proteins recruited to the mRNA during splicing. Intron enhanced expression of Cas9 may also allow use of less AAV vector doses for in vivo gene editing. In addition, introns allow the use of PAM sites recognized by different Cas9 orthologues, as well as protospacer-like sequences recognized by different DNA-targeting nucleic acids, making SIN vector systems readily adaptable for use with Cas9 orthologues. In certain aspects, introns that can be used in the expression constructs described herein include, but are not limited to, SEQ ID NOs: 113, 117 or 119. SIN sites may be inserted into these introns at various locations, which may or may not include deletion of one or more nucleotides in the intronic sequence. For example, an intron containing a SIN site can be SEQ ID NOs: 114-115, SEQ ID NO: 118, or SEQ ID NO: 120. SEQ ID NO: 116 shows a representative self-inactivating chimeric intron that may be used to swap out SIN sites, where N represents nucleotides of a selected SIN site.

In some examples, a nucleic acid sequence encoding a promoter can be operably linked to the first segment.

In some examples, the site-directed polypeptide can be Cas9, Cpf1, or any variants thereof. In other examples, the site directed polypeptide can be Streptococcus pyogenes Cas9 (SpCas9) or any variants thereof. In other examples, the site directed polypeptide can be Campylobacter jejuni Cas9 (CjCas9) or any variants thereof. In other examples, the site directed polypeptide can be Staphylococcus aureus Cas9 (SaCas9) or any variants thereof. The SaCas9 can comprise a nucleotide sequence encoding the amino acid sequence set forth in SEQ ID NO: 1. SaCas9 can comprise a nucleotide sequence as set forth in SEQ ID NO: 79, or codon optimized variants thereof. The SaCas9 variant can comprise a D10A mutation in the amino acid sequence set forth in SEQ ID NO: 2. The Cas9 variant can comprise an N580A mutation in the amino acid sequence set forth in SEQ ID NO: 3. The SaCas9 variant can comprise both a D10A mutation and an N580A mutation in the amino acid sequence set forth in SEQ ID NO: 4.

In some examples, the DNA-targeting nucleic acid can be a guide RNA (gRNA) or single-molecule guide RNA (sgRNA). The gRNA or sgRNA can be synthesized inside the cells or be delivered from outside the cells as synthetic sgRNA or synthetic dual gRNAs. The gRNA or sgRNA can also be partly synthesized and partly delivered from outside of the cell.

In some examples, one or more third segments can comprise a SIN site. In some examples, one or more third segments can comprise a protospacer adjacent motif (PAM). In other examples, the PAM can be NNGRRN or any variants thereof (e.g.: NNGRRT, NNGRRV). In other examples, the PAM can be NNGRYT, or NNGYRT, or any variants thereof (Friedland et al., 2015, Genome Biology, 16(257):1-10). In some examples, one or more third segments can comprise a DNA-target.

In some examples, one or more third segments can be located at any one or more of: a 5′ end of the first segment, upstream of the start codon and/or downstream of the transcriptional start site; within one or more naturally occurring or chimeric inserted introns; or a 3′ end of the first segment between the stop codon and poly(A) termination site.

In some examples, the third segment is not fully complementary to the second segment in at least one, two, three, four, five or more locations along the length of the third segment.

In some examples, the third segment is not fully complementary to the second segment. In some examples, the third segment is not fully complementary to the second segment and (1) differs in sequence at one, two, three or more bases and (2) differs in length with one or more bulges from extra bases in the guide or target DNA sequences.

In some examples, the third segment is not fully complementary to the nucleotide sequence of the DNA-targeting nucleic acid in at least one location. In other examples, the third segment is not fully complementary to the nucleotide sequence of the DNA-targeting nucleic acid in at least two locations. In other examples, the third segment is not fully complementary to the nucleotide sequence of the DNA-targeting nucleic acid in at least three, four, five or more locations.

In some examples, the third segment has a canonical protospacer adjacent motif (PAM), such as NGG, or has an alternative PAM. An example of an alternative PAM for the SpCas9 is NAG. In some examples, the third segment has a PAM proceeded by a bulge, such as NNGG (N can be any nucleotide, including wild-type).

In some examples, the third segment has a canonical protospacer adjacent motif (PAM) for one or more orthologue Cas9, such as NNGRRT, or has an alternative PAM, such as NNGRRN, NNGRYT, NNGYRT, NNGRRV.

In some examples, the third segment has a canonical protospacer adjacent motif (PAM) for one or more orthologue Cas9, such as, NNNNACA or has an alternative PAM, such as NNNACAC, NNVRYAC, or NNNVRYM.

In some examples, the site-directed polypeptide can be S. pyogenes (Sp) Cas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site.

In some examples, the site-directed polypeptide can be SpCas9 and the DNA-targeting nucleic acid can be a gRNA or sgRNA that targets the one or more third segments, wherein the one or more third segments is located within one or more naturally occurring or chimeric inserted introns.

In some examples, the site-directed polypeptide can be SpCas9 and the DNA-targeting nucleic acid can be a gRNA or sgRNA that targets the one or more third segments, wherein the one or more third segments is located at the 3′ end of the first segment between the stop codon and poly(A) termination site.

In some examples, the site-directed polypeptide can be SpCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and at the 3′ end of the first segment between the stop codon and poly(A) termination site.

In some examples, the site-directed polypeptide can be SpCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and within one or more naturally occurring or chimeric inserted introns.

In some examples, the site-directed polypeptide can be SpCas9 and the DNA-targeting nucleic acid can be a gRNA or sgRNA that targets the one or more third segments, wherein the one or more third segments is located at the 3′ end of the first segment between the stop codon and poly(A) termination site; and within one or more naturally occurring or chimeric inserted introns.

In some examples, the site-directed polypeptide can be SpCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; at the 3′ end of the first segment between the stop codon and poly(A) termination site; and within one or more naturally occurring or chimeric inserted introns.

In some examples, the site-directed polypeptide can be C. jejuni (Cj) Cas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site.

In some examples, the site-directed polypeptide can be CjCas9 and the DNA-targeting nucleic acid can be a gRNA or sgRNA that targets the one or more third segments, wherein the one or more third segments is located within one or more naturally occurring or chimeric inserted introns.

In some examples, the site-directed polypeptide can be CjCas9 and the DNA-targeting nucleic acid can be a gRNA or sgRNA that targets the one or more third segments, wherein the one or more third segments is located at the 3′ end of the first segment between the stop codon and poly(A) termination site.

In some examples, the site-directed polypeptide can be CjCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and at the 3′ end of the first segment between the stop codon and poly(A) termination site.

In some examples, the site-directed polypeptide can be CjCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and within one or more naturally occurring or chimeric inserted introns.

In some examples, the site-directed polypeptide can be CjCas9 and the DNA-targeting nucleic acid can be a gRNA or sgRNA that targets the one or more third segments, wherein the one or more third segments is located at the 3′ end of the first segment between the stop codon and poly(A) termination site; and within one or more naturally occurring or chimeric inserted introns.

In some examples, the site-directed polypeptide can be CjCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; at the 3′ end of the first segment between the stop codon and poly(A) termination site; and within one or more naturally occurring or chimeric inserted introns.

In some examples, the site-directed polypeptide can be S. aureus (Sa) Cas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site.

In some examples, the site-directed polypeptide can be SaCas9 and the DNA-targeting nucleic acid can be a gRNA or sgRNA that targets the one or more third segments, wherein the one or more third segments is located within one or more naturally occurring or chimeric inserted introns.

In some examples, the site-directed polypeptide can be SaCas9 and the DNA-targeting nucleic acid can be a gRNA or sgRNA that targets the one or more third segments, wherein the one or more third segments is located at the 3′ end of the first segment between the stop codon and poly(A) termination site.

In some examples, the site-directed polypeptide can be SaCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and at the 3′ end of the first segment between the stop codon and poly(A) termination site.

In some examples, the site-directed polypeptide can be SaCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and within one or more naturally occurring or chimeric inserted introns.

In some examples, the site-directed polypeptide can be SaCas9 and the DNA-targeting nucleic acid can be a gRNA or sgRNA that targets the one or more third segments, wherein the one or more third segments is located at the 3′ end of the first segment between the stop codon and poly(A) termination site; and within one or more naturally occurring or chimeric inserted introns.

In some examples, the site-directed polypeptide can be SaCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; at the 3′ end of the first segment between the stop codon and poly(A) termination site; and within one or more naturally occurring or chimeric inserted introns.

In some examples, the third segment of the self-inactivating CRISPR/Cas or CRISPR/Cpf1 system comprises a nucleotide sequence that is less than 100 nucleotides in length (e.g., less than 75, less than 50, less than 25 nucleotides in length; or ranging from about 20-50, 20-75, 25-100, 75-100, or 50-75 nucleotides in length). In some examples, the third segment comprises a nucleotide sequence that is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length.

The first segment, the second segment, and the third segment of the self-inactivating CRISPR/Cas or CRISPR/Cpf1 system, can be delivered via one or more vectors. For example, the first segment, the second segment, and the third segment of the self-inactivating CRISPR/Cas or CRISPR/Cpf1 system can be delivered via the same vector. In another example, the first segment and the third segment can be provided together in a first vector and the second segment can be provided in a second vector. The third segment can be present in the vector at a location 5′ of the first segment. The third segment can be present in the vector at a location 3′ of the first segment. The one or more third segments can be present in the vector at the 5′ and 3′ ends of the first segment. The one or more third segments can be present in the vector within the first segment, for example, within introns of the first segment.

The vector can be one or more adeno-associated virus (AAV) vectors. The adeno-associated virus (AAV) vector can be AAV2. The adeno-associated virus (AAV) vector can be AAV1-AAV9, or any variants thereof.

When provided by a separate vector, the second segment can be administered sequentially or simultaneously with the vector encoding the first segment and the third segment. When administered sequentially, the vector encoding the second segment is delivered after the vector encoding the first segment and the third segment to allow for the intended gene editing or gene engineering to occur. This period can be a period of minutes (e.g. 5 minutes, 10 minutes, 20 minutes, 30 minutes, 45 minutes, 60 minutes), hours (e.g. 2 hours, 4 hours, 6 hours, 8 hours, 12 hours, 24 hours), days (e.g. 2 days, 3 days, 4 days, 7 days), weeks (e.g. 2 weeks, 3 weeks, 4 weeks), months (e.g. 2 months, 4 months, 8 months, 12 months) or years (2 years, 3 years, 4 years). In this regard, the site-directed polypeptide can associate with a first gRNA/sgRNA capable of hybridizing to a target gene sequence, such as a genomic locus or loci of interest and undertakes the function(s) desired of the CRISPR/Cas or CRISPR/Cpf1 system (e.g., gene engineering); and subsequently the site-directed polypeptide can then associate with the third segment capable of hybridizing to the sequence comprising a nucleotide sequence that encodes at least part of the site-directed polypeptide or guide RNA targeting the target DNA. Where the third segment targets the nucleotide sequence encoding expression of the site-directed polypeptide, the enzyme becomes impeded and the system becomes self-inactivating. In various example, CRISPR RNA that targets site-directed polypeptide expression applied via, for example liposome, lipofection, nanoparticles, microvesicles as explained herein, can be administered sequentially or simultaneously.

In some aspects, a third segment comprising a SIN site can be provided that is located downstream of a site-directed polypeptide start codon. A gRNA is capable of hybridizing to the SIN site whereby after a period of time there is a mutation in the coding sequence of the site-directed polypeptide and/or loss of the site-directed polypeptide expression. In some aspects, one or more SIN site(s) are provided that are located 5′ and 3′ of site-directed polypeptide ORF. A gRNA is capable of hybridizing to the one or more SIN sites, whereby after a period of time there is an inactivation of the site-directed polypeptide.

### Pharmaceutical Compositions

The CRISPR/Cas or CRISPR/Cpf1 and self-inactivating CRISPR/Cas or CRISPR/Cpf1 systems described herein can be formulated into pharmaceutical compositions by combination with appropriate pharmaceutically acceptable carriers or diluents.

Exemplary pharmaceutically acceptable excipients such as carriers, solvents, stabilizers, adjuvants, diluents, etc., depending upon the particular mode of administration and dosage form. Contemplated pharmaceutical compositions can be generally formulated to achieve a physiologically compatible pH, and range from a pH of about 3 to a pH of about 11, about pH 3 to about pH 7, depending on the formulation and route of administration. In alternative examples, the pH can be adjusted to a range from about pH 5.0 to about pH 8. In some examples, the compositions comprise a therapeutically effective amount of at least one compound as described herein, together with one or more pharmaceutically acceptable excipients.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Pharmaceutical compositions can be formulated into preparations in solid, semi-solid, liquid or gaseous forms, such as tablets, capsules, powders, granules, ointments, solutions, suppositories, injections, inhalants, gels, microspheres, and aerosols. As such, administration of a guide RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent can be systemic after administration or can be localized using regional administration, intramural administration, or use of an implant that acts to retain the active dose at the site of implantation. The active agent can be formulated for immediate activity or it can be formulated for sustained release.

In some cases, the components of the composition are individually pure, e.g., each of the components is at least about 75%, at least about 80%, at least about 90%, at least about 95%, at least about 98%, at least about 99%, or at least 99%, pure. In some cases, the individual components of a composition are pure before being added to the composition.

For some conditions, particularly central nervous system conditions, it can be necessary to formulate agents to cross the blood-brain barrier (BBB). One strategy for drug delivery through the BBB entails disruption of the BBB, either by osmotic means such as mannitol or leukotrienes, or biochemically using vasoactive substances such as bradykinin. The potential for using BBB opening to target specific agents to brain tumors can also be an option. A BBB disrupting agent can be co-administered with the therapeutic compositions of the invention when the compositions are administered by intravascular injection. Other strategies to go through the BBB can entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties can also be conjugated to the therapeutic compounds for use in the invention to facilitate transport across the endothelial wall of the blood vessel. Alternatively, drug delivery of therapeutics agents behind the BBB can be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversably affixed (see e.g. US Application Nos. 20080081064 and 20090196903, incorporated herein by reference).

Typically, an effective amount of a self-inactivating CRISPR/Cas or CRISPR/Cpf1 system comprising a guide RNA and/or site-directed modifying polypeptide and/or donor polynucleotide can be provided. The amount of recombination can be measured by any convenient method, e.g. as described above and known in the art. The calculation of the effective amount or effective dose of a self-inactivating CRISPR/Cas or CRISPR/Cpf1 system comprising a guide RNA and/or site-directed modifying polypeptide and/or donor polynucleotide to be administered is within the skill of one of ordinary skill in the art, and can be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose can be more than an intrathecally administered dose, given the greater body of fluid into which the therapeutic composition is being administered. Similarly, compositions which are rapidly cleared from the body can be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a self-inactivating CRISPR/Cas or CRISPR/Cpf1 system comprising a guide RNA and/or site-directed modifying polypeptide and/or donor polynucleotide can be obtained from a suitable commercial source. As a general proposition, the total pharmaceutically effective amount of a guide RNA and/or site-directed modifying polypeptide and/or donor polynucleotide administered parenterally per dose will be in a range that can be measured by a dose response curve.

Therapies based on a self-inactivating CRISPR/Cas or CRISPR/Cpf1 system comprising a guide RNA and/or site-directed modifying polypeptide and/or donor polynucleotides, i.e. preparations of a guide RNA and/or site-directed modifying polypeptide and/or donor polynucleotide to be used for therapeutic administration, must be sterile. Sterility is readily accomplished by filtration through sterile filtration membranes (e.g., 0.2 μm membranes). Therapeutic compositions can be generally placed into a container having a sterile access port, for example, an intravenous solution bag or vial having a stopper pierceable by a hypodermic injection needle. The therapies based on a self-inactivating CRISPR/Cas or CRISPR/Cpf1 system comprising a guide RNA and/or site-directed modifying polypeptide and/or donor polynucleotide can be stored in unit or multi-dose containers, for example, sealed ampules or vials, as an aqueous solution or as a lyophilized formulation for reconstitution. As an example of a lyophilized formulation, 10-m1 vials are filled with 5 ml of sterile-filtered 1% (w/v) aqueous solution of compound, and the resulting mixture is lyophilized. The infusion solution can be prepared by reconstituting the lyophilized compound using bacteriostatic Water-for-Injection.

### Kits

The present disclosure provides kits for carrying out the methods described herein. A kit can include one or more of a DNA-targeting nucleic acid, a polynucleotide encoding a DNA-targeting nucleic acid, a site-directed polypeptide, a polynucleotide encoding a site-directed polypeptide, and/or any nucleic acid or proteinaceous molecule necessary to carry out the aspects of the methods described herein, or any combination thereof.

A kit comprising a self-inactivating CRISPR/Cas or CRISPR/Cpf1 system can comprise: (1) a vector comprising (i) a nucleotide sequence encoding a DNA-targeting nucleic acid (ii) nucleotide sequence encoding a site-directed polypeptide, and (iii) a nucleotide sequence that is substantially complementary to the nucleotide sequence encoding the DNA-targeting nucleic acid, and (2) a reagent for reconstitution and/or dilution of the vector(s).

A kit comprising a self-inactivating CRISPR/Cas or CRISPR/Cpf1 system can comprise: (1) a vector comprising (i) a nucleotide sequence encoding a site-directed polypeptide, and (ii) a nucleotide sequence that is substantially complementary to the nucleotide sequence encoding the site-directed polypeptide and (2) a vector comprising (i) a nucleotide sequence encoding a DNA-targeting nucleic acid, (3) a reagent for reconstitution and/or dilution of the vector.

A kit comprising a self-inactivating CRISPR/Cas or CRISPR/Cpf1 system can comprise: (1) a vector comprising (i) a nucleotide sequence encoding a DNA-targeting nucleic acid, and (ii) a nucleotide sequence that is substantially complementary to the nucleotide sequence encoding the DNA-targeting nucleic acid and (2) a vector comprising (i) a nucleotide sequence encoding a site-directed polypeptide, (3) a reagent for reconstitution and/or dilution of the vector.

The kit can comprise a single-molecule guide DNA-targeting nucleic acid. In some examples, the kit can comprise a double-molecule DNA-targeting nucleic acid. In some examples, the kit can comprise two or more double-molecule guides or single-molecule guides. In some examples, the kits can comprise a vector that encodes the nucleic acid targeting nucleic acid.

In some examples, the kit can further comprise a polynucleotide to be inserted to effect the desired genetic modification.

Components of a kit can be in separate containers, or combined in a single container.

Any kit described above can further comprise one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the endonuclease, or improve the specificity of targeting.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that comprises a web address where the instructions can be viewed and/or from which the instructions can be downloaded. As with the instructions, this means for obtaining the instructions can be recorded on a suitable substrate.

### Methods of Controlling Cas9 of Cpf1 Expression

In some examples, a method of controlling gene expression can comprise contacting a cell with any of the self-inactivating CRISPR/Cas or CRISPR/Cpf1 systems disclosed herein. In other examples, the method of controlling gene expression can further comprise transforming the cell with a third vector comprising a nucleotide sequence encoding a homology-directed repair (HDR) template.

### Methods of Genetically Modifying a Cell

In some examples, a method of genetically modifying a cell can comprise introducing to a cell or contacting a cell with any of the self-inactivating CRISPR/Cas or CRISPR/Cpf1 systems disclosed herein.

### Nucleic Acids for Use in a Self-Inactivating CRISPR/Cas or CRISPR/Cpf1 Systems

In some examples, a nucleic acid for use in any of the self-inactivating CRISPR/Cas or CRISPR/Cpf1 systems disclosed herein can comprise a codon modified, or codon optimized sequence encoding a site-directed polypeptide. The codon optimized sequence can further comprise a SIN site. The SIN site can comprise the PAM, NNGRRT, or variants thereof. The SIN site can comprise a sequence selected from the group consisting of SEQ ID NOs: 63-72. The codon optimized sequence can comprise SEQ ID NO: 79.

In some examples, a method of controlling gene expression can comprise contacting a cell with any of the self-inactivating CRISPR/Cas or CRISPR/Cpf1 systems disclosed herein. In other examples, the method of controlling gene expression can further comprise transforming the cell with a third vector comprising a nucleotide sequence encoding a homology-directed repair (HDR) template.

### Systems, Methods, and Compositions of the Invention

Accordingly, the present disclosure relates in particular to the following non-limiting inventions: In a first system, System 1, the present disclosure provides a self-inactivating CRISPR-Cas system comprising: a first segment comprising a nucleotide sequence that encodes a site-directed polypeptide, a second segment comprising a nucleotide sequence that encodes a DNA-targeting nucleic acid; and one or more third segments comprising a nucleotide sequence that is substantially complementary to the nucleotide sequence of the DNA-targeting nucleic acid.

In another system, System 2, the present disclosure provides the self-inactivating CRISPR-Cas system of System 1, wherein the site-directed polypeptide is Cas9 or any variants thereof.

In another system, System 3, the present disclosure provides the self-inactivating CRISPR-Cas system of System 1, wherein the site directed polypeptide is Staphylococcus aureus Cas9 (SaCas9) or any variants thereof, Streptococcus pyogenes Cas9 (SpCas9) or any variants thereof, or Campylobacter jejuni Cas9 (CjCas9) or any variants thereof.

In another system, System 4, the present disclosure provides the self-inactivating CRISPR-Cas system of any of Systems 1-3, wherein the DNA-targeting nucleic acid is a guide RNA (gRNA) or single-molecule guide RNA (sgRNA).

In another system, System 5, the present disclosure provides the self-inactivating CRISPR-Cas system of any of Systems 1-4, wherein the one or more third segments comprise a SIN site.

In another system, System 6, the present disclosure provides the self-inactivating CRISPR-Cas system of any of Systems 1-4, where the one or more third segments comprise a protospacer adjacent motif (PAM).

In another system, System 7, the present disclosure provides the self-inactivating CRISPR-Cas system of System 6, wherein the PAM is: NNGRRT, NNGRRN, NNGRYT, NNGYRT, NNGRRV, or any variants thereof; or NRG or any variants thereof; or NNNNACA, NNNACAC, NNVRYAC, or NNNVRYM, or any variants thereof.

In another system, System 8, the present disclosure provides the self-inactivating CRISPR-Cas system of any of Systems 1-7, wherein the first segment comprising a nucleotide sequence that encodes a site-directed polypeptide, further comprises a start codon, a stop codon, and a poly(A) termination site.

In another system, System 9, the present disclosure provides the self-inactivating CRISPR-Cas system of System 8, wherein the nucleic acid that encodes the site-directed polypeptide, further comprises one or more naturally occurring or chimeric introns inserted into, upstream, and/or downstream of a Cas9 open reading frame (ORF).

In another system, System 10, the present disclosure provides the self-inactivating CRISPR-Cas system of any of Systems 8-9, wherein the one or more third segments are located at any one or more of: a) a 5′ end of the first segment, upstream of the start codon and/or downstream of the transcriptional start site; b) within one or more naturally occurring or chimeric inserted introns; or c) a 3′ end of the first segment between the stop codon and poly(A) termination site.

In another system, System 11, the present disclosure provides the self-inactivating CRISPR-Cas system of System 10, wherein the site-directed polypeptide is SaCas9 and the DNA-targeting nucleic acid is a guide RNA (gRNA) or single-molecule guide RNA (sgRNA) that targets the one or more third segments, wherein the one or more third segments is located at the 5′ end of the first segment, upstream of the start codon and/or downstream of the transcriptional start site.

In another system, System 12, the present disclosure provides the self-inactivating CRISPR-Cas system of System 10, wherein the site-directed polypeptide is SaCas9 and the DNA-targeting nucleic acid is a guide RNA (gRNA) or single-molecule guide RNA (sgRNA) that targets the one or more third segments, wherein the one or more third segments is located within one or more naturally occurring or chimeric inserted introns.

In another system, System 13, the present disclosure provides the self-inactivating CRISPR-Cas system of System 10, wherein the site-directed polypeptide is SaCas9 and the DNA-targeting nucleic acid is a guide RNA (gRNA) or single-molecule guide RNA (sgRNA) that targets the one or more third segments, wherein the one or more third segments is located at the 3′ end of the first segment between the stop codon and poly(A) termination site.

In another system, System 14, the present disclosure provides the self-inactivating CRISPR-Cas system of System 10, wherein the site-directed polypeptide is SaCas9 and the DNA-targeting nucleic acid is a guide RNA (gRNA) or single-molecule guide RNA (sgRNA) that targets the one or more third segments, wherein the one or more third segments is located at the 5′ end of the first segment, upstream of the start codon and/or downstream of the transcriptional start site; and at the 3′ end of the first segment between the stop codon and poly(A) termination site.

In another system, System 15, the present disclosure provides the self-inactivating CRISPR-Cas system of System 10, wherein the site-directed polypeptide is SaCas9 and the DNA-targeting nucleic acid is a guide RNA (gRNA) or single-molecule guide RNA (sgRNA) that targets the one or more third segments, wherein the one or more third segments is located at the 5′ end of the first segment, upstream of the start codon and/or downstream of the transcriptional start site; and within one or more naturally occurring or chimeric inserted introns.

In another system, System 16, the present disclosure provides the self-inactivating CRISPR-Cas system of System 10, wherein the site-directed polypeptide is SaCas9 and the DNA-targeting nucleic acid is a guide RNA (gRNA) or single-molecule guide RNA (sgRNA) that targets the one or more third segments, wherein the one or more third segments is located at the 3′ end of the first segment between the stop codon and poly(A) termination site; and within one or more naturally occurring or chimeric inserted introns.

In another system, System 17, the present disclosure provides the self-inactivating CRISPR-Cas system of System 10, wherein the site-directed polypeptide is SaCas9 and the DNA-targeting nucleic acid is a guide RNA (gRNA) or single-molecule guide RNA (sgRNA) that targets the one or more third segments, wherein the one or more third segments is located at the 5′ end of the first segment, upstream of the start codon and/or downstream of the transcriptional start site; at the 3′ end of the first segment between the stop codon and poly(A) termination site; and within one or more naturally occurring or chimeric inserted introns.

In another system, System 18, the present disclosure provides the self-inactivating CRISPR-Cas system of any of Systems 1-17, wherein the first segment and the third segment are provided together in a first vector and the second segment is provided in a second vector.

In another system, System 19, the present disclosure provides the self-inactivating CRISPR-Cas system of any of Systems 1-17, wherein the first segment, second segment, and third segment are provided together in a vector.

In another system, System 20, the present disclosure provides the self-inactivating CRISPR-Cas system of any of Systems 18-19, wherein the third segment is present in the first or second vector at a location 5′ of the first segment.

In another system, System 21, the present disclosure provides the self-inactivating CRISPR-Cas system of any of Systems 18-19, wherein the third segment is present in the first or second vector at a location 3′ of the first segment.

In another system, System 22, the present disclosure provides the self-inactivating CRISPR-Cas system of any of Systems 18-19, wherein the one or more third segments are present in the first or second vector at the 5′ and 3′ ends of the first segment.

In another system, System 23, the present disclosure provides the self-inactivating CRISPR-Cas system of any of Systems 1-22, wherein the third segment is less than 100 nucleotides in length.

In another system, System 24, the present disclosure provides the self-inactivating CRISPR-Cas system of System 23, wherein the third segment is less than 50 nucleotides in length.

In another system, System 25, the present disclosure provides the self-inactivating CRISPR-Cas system of System 23, wherein the third segment is less than 25 nucleotides in length.

In another system, System 26, the present disclosure provides the self-inactivating CRISPR-Cas system of any of Systems 1-25, wherein the third segment is not fully complementary to the nucleotide sequence of the DNA-targeting nucleic acid in at least one location.

In another system, System 27, the present disclosure provides the self-inactivating CRISPR-Cas system of any of Systems 1-26, wherein the third segment is not fully complementary to the nucleotide sequence of the DNA-targeting nucleic acid in at least two locations.

In another system, System 28, the present disclosure provides the self-inactivating CRISPR-Cas system of any of Systems 1-27, wherein a nucleic acid sequence encoding a promoter is operably linked to the first segment.

In another system, System 29, the present disclosure provides the self-inactivating CRISPR-Cas system of System 28, wherein the promoter is a spatially-restricted promoter, bidirectional promoter driving gRNA in one direction and Cas9 in the opposite orientation, or an inducible promoter.

In another system, System 30, the present disclosure provides the self-inactivating CRISPR-Cas system of System 29, wherein the spatially-restricted promoter is selected from the group consisting of: any tissue or cell type specific promoter, a hepatocyte-specific promoter, a neuron-specific promoter, an adipocyte-specific promoter, a cardiomyocyte-specific promoter, a skeletal muscle-specific promoter, a lung progenitor cell specific promoter, a photoreceptor-specific promoter, and a retinal pigment epithelial (RPE) selective promoter.

In another system, System 31, the present disclosure provides the self-inactivating CRISPR-Cas system of System 3, wherein Cas9 comprises a nucleotide sequence encoding a Cas9 protein as set forth in SEQ ID NO. 1, wherein the SaCas9 comprises a nucleotide sequence as set forth in SEQ ID NO: 79.

In another system, System 32, the present disclosure provides the self-inactivating CRISPR-Cas system of System 2, wherein the Cas9 variant comprises a D10A mutation in the amino acid sequence set forth in SEQ ID NO: 2.

In another system, System 33, the present disclosure provides the self-inactivating CRISPR-Cas system of System 2, wherein the Cas9 variant comprises an N580A mutation in the amino acid sequence set forth in SEQ ID NO: 3.

In another system, System 34, the present disclosure provides the self-inactivating CRISPR-Cas system of System 2, wherein the Cas9 variant comprises both a D10A mutation and an N580A mutation in the amino acid sequence set forth in SEQ ID NO: 4.

In another system, System 35, the present disclosure provides the self-inactivating CRISPR-Cas system of any of Systems 18-19, wherein the vector is one or more adeno-associated virus (AAV) vectors.

In another system, System 36, the present disclosure provides the self-inactivating CRISPR-Cas system of System 35, wherein the adeno-associated virus (AAV) vector is AAV2.

In another system, System 37, the present disclosure provides a self-inactivating CRISPR-Cas system comprising: a first segment comprising a nucleotide sequence that encodes a site-directed polypeptide; and a second segment comprising a nucleotide sequence that encodes a DNA-targeting nucleic acid; wherein the nucleotide sequence of the first segment comprises a SIN site that is substantially complementary to a DNA-targeting segment of the DNA-targeting nucleic acid.

In another system, System 38, the present disclosure provides the self-inactivating CRISPR-Cas system of System 37, wherein the site-directed polypeptide is Cas9 or any variants thereof.

In another system, System 39, the present disclosure provides the self-inactivating CRISPR-Cas system of System 37, wherein the site-directed polypeptide is Staphylococcus aureus Cas9 (SaCas9), Streptococcus pyogenes Cas9 (SpCas9), Campylobacter jejuni Cas9 (CjCas9), or any variants thereof

In another system, System 40, the present disclosure provides the self-inactivating CRISPR-Cas system of System 37, wherein the site-directed polypeptide is encoded by a sequence that is 90% identical to a nucleotide sequence that encodes wild-type SaCas9.

In another system, System 41, the present disclosure provides the self-inactivating CRISPR-Cas system of any of Systems 37-40, wherein the DNA-targeting nucleic acid is a guide RNA (gRNA) or single-molecule guide RNA (sgRNA).

In another system, System 42, the present disclosure provides the self-inactivating CRISPR-Cas system of System 41, wherein the gRNA or sgRNA comprises a sequence selected from the group consisting of SEQ ID NOs: 80 to 91.

In another system, System 43, the present disclosure provides the self-inactivating CRISPR-Cas system of System 41, wherein the sgRNA comprises a sequence selected from the group consisting of SEQ ID NOs: 74-78.

In another system, System 44, the present disclosure provides the self-inactivating CRISPR-Cas system of any of Systems 37-43, wherein the first segment comprising a nucleotide sequence that encodes a site-directed polypeptide, further comprises: a start codon, a stop codon, and a poly(A) termination site.

In another system, System 45, the present disclosure provides the self-inactivating CRISPR-Cas system of System 44, wherein the SIN site is located between the start codon and the stop codon.

In another system, System 46, the present disclosure provides the self-inactivating CRISPR-Cas system of any of Systems 37-45, wherein the SIN site comprises a sequence selected from the group consisting of SEQ ID NO: 63-72.

In another system, System 47, the present disclosure provides the self-inactivating CRISPR-Cas system of any of System 37-46, wherein the first segment is provided in a first vector and the second segment is provided in a second vector.

In another system, System 48, the present disclosure provides the self-inactivating CRISPR-Cas system of any of System 37-46, wherein the first segment and second segment are provided together in a vector.

In another system, System 49, the present disclosure provides the self-inactivating CRISPR-Cas system of any of Systems 37-48, wherein the DNA-targeting segment of a DNA-targeting nucleic acid is not fully complementary to the nucleotide sequence of the SIN site in at least one location.

In another system, System 50, the present disclosure provides the self-inactivating CRISPR-Cas system of any of Systems 37-48, wherein the DNA-targeting segment of a DNA-targeting nucleic acid is not fully complementary to the nucleotide sequence of the SIN site in at least two locations.

In another system, System 51, the present disclosure provides the self-inactivating CRISPR-Cas system of any of Systems 37-50, wherein a nucleic acid sequence encoding a promoter is operably linked to the first segment.

In another system, System 52, the present disclosure provides the self-inactivating CRISPR-Cas system of System 51, wherein the promoter is a spatially-restricted promoter, bidirectional promoter driving gRNA in one direction and Cas9 in the opposite orientation, or an inducible promoter.

In another system, System 53, the present disclosure provides the self-inactivating CRISPR-Cas system of System 52, wherein the spatially-restricted promoter is selected from the group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a retinal pigment epithelial (RPE) selective promoter.

In another system, System 54, the present disclosure provides the self-inactivating CRISPR-Cas system of System 37, wherein the first segment comprises a nucleotide sequence encoding a Cas9 protein comprising an amino acid sequence selected from the group consisting of SEQ ID NOs: 1-4.

In another system, System 55, the present disclosure provides the self-inactivating CRISPR-Cas system of System 37, wherein the first segment comprises a nucleotide sequence encoding a Cas9 protein comprising the amino acid sequence of SEQ ID NO: 1.

In another system, System 56, the present disclosure provides the self-inactivating CRISPR-Cas system of System 38, wherein the Cas9 variant comprises a D10A mutation in the amino acid sequence set forth in SEQ ID NO: 2.

In another system, System 57, the present disclosure provides the self-inactivating CRISPR-Cas system of System 38, wherein the Cas9 variant comprises an N580A mutation in the amino acid sequence set forth in SEQ ID NO: 3.

In another system, System 58, the present disclosure provides the self-inactivating CRISPR-Cas system of System 38, wherein the Cas9 variant comprises both a D10A mutation and an N580A mutation in the amino acid sequence set forth in SEQ ID NO: 4.

In another system, System 59, the present disclosure provides the self-inactivating CRISPR-Cas system of any of Systems 47-48, wherein the vector is one or more adeno-associated virus (AAV) vectors.

In another system, System 60, the present disclosure provides the self-inactivating CRISPR-Cas system of System 59, wherein the adeno-associated virus (AAV) vector is AAV2.

In another system, System 61, the present disclosure provides a self-inactivating CRISPR-Cas system comprising: (a) a first nucleic acid segment comprising a codon optimized nucleotide sequence encoding a site-directed Cas9 polypeptide or variant thereof, wherein the codon optimized sequence comprises a first self-inactivating (SIN) site and an adjacent Protospacer Adjacent Motif (PAM) within the open reading frame (ORF), and wherein the first SIN site is the result of codon optimization; and (b) a second nucleic acid segment comprising a nucleotide sequence encoding a guide RNA (gRNA), wherein the gRNA comprises a DNA-targeting sequence that is complementary to the self-inactivating (SIN) site in the first nucleic acid segment, wherein the gRNA guides the Cas9 polypeptide or variant thereof to cleave the first nucleic acid segment at the SIN site within the codon optimized sequence and reduces expression of the site directed Cas9 polypeptide or variant thereof.

In another system, System 62, the present disclosure provides the self-inactivating CRISPR-Cas system of System 61, wherein the first nucleic acid segment comprises a nucleotide sequence encoding a first target gRNA that comprises a DNA-targeting sequence that is complementary to a nucleotide sequence present in a target gene in a cell.

In another system, System 63, the present disclosure provides the self-inactivating CRISPR-Cas system of System 63, wherein the first nucleic acid segment comprises a nucleotide sequence that encodes a second target gRNA that comprises a DNA-targeting that is complementary to a nucleotide sequence present in a target gene in a cell.

In another system, System 64, the present disclosure provides the self-inactivating CRISPR-Cas system of System 62 or System 63, wherein the first SIN site in the first nucleic acid segment is located (a) at the 5′ end of the nucleotide sequence encoding the Cas9 polypeptide or variant thereof; (b) at the 3′ end of the nucleotide sequence encoding the Cas9 polypeptide or variant thereof; or (c) in an intron within the nucleotide sequence encoding the Cas9 polypeptide or variant thereof.

In another system, System 65, the present disclosure provides the self-inactivating CRISPR-Cas system of any one of Systems 62-64, wherein the nucleotide sequence encoding the first and/or second target gRNA in the first nucleic acid segment is located: (a) at the 5′ end of the nucleotide sequence encoding the Cas9 polypeptide or variant thereof; or (b) at the 3′ end of the nucleotide sequence encoding the Cas9 polypeptide or variant thereof.

In another system, System 66, the present disclosure provides the self-inactivating CRISPR-Cas system of any one of Systems 63-65, wherein the nucleotide sequence encoding the first target gRNA is located at the 5′ end of the nucleotide sequence encoding the Cas9 polypeptide or variant thereof, and the second target gRNA is located at the 3′ end of the of the nucleotide sequence encoding the site-directed Cas9 polypeptide or variant thereof.

In another system, System 67, the present disclosure provides the self-inactivating CRISPR-Cas system of any one of Systems 61-66, wherein the codon optimized sequence comprises a second SIN site, and wherein the nucleotide sequence of the first SIN site and the second SIN site are the same.

In another system, System 68, the present disclosure provides the self-inactivating CRISPR-Cas system any one of Systems 61-66, wherein the codon optimized sequence comprises a second SIN site, and wherein the nucleotide sequence of the first target SIN site and the second target SIN site are different.

In another system, System 69, the present disclosure provides the self-inactivating CRISPR-Cas system any one of Systems 61-68, wherein the gRNA is a two-molecule guide RNA.

In another system, System 70, the present disclosure provides the self-inactivating CRISPR-Cas system of System 69, wherein the two-molecule gRNA comprises a CRISPR RNA (crRNA-like) molecule and a trans-activating CRISPR RNA (tracrRNA-like) molecule.

In another system, System 71, the present disclosure provides the self-inactivating CRISPR-Cas system of any one of Systems 61-68, wherein the gRNA is a single RNA molecule.

In another system, System 72, the present disclosure provides the self-inactivating CRISPR-Cas system of any one of Systems 62-71, wherein the first target gRNA is a two-molecule guide RNA.

In another system, System 73, the present disclosure provides the self-inactivating CRISPR-Cas system of System 72, wherein the first target gRNA comprises a CRISPR RNA (crRNA-like) molecule and a trans-activating CRISPR RNA (tracrRNA-like) molecule.

In another system, System 74, the present disclosure provides the self-inactivating CRISPR-Cas system of any one of Systems 62-71, wherein the first target gRNA is a single RNA molecule.

In another system, System 75, the present disclosure provides the self-inactivating CRISPR-Cas system of any one of Systems 62-74, wherein the second target gRNA is a two-molecule guide RNA.

In another system, System 76, the present disclosure provides the self-inactivating CRISPR-Cas system of System 75, wherein the second target gRNA comprises a CRISPR RNA (crRNA-like) molecule and a trans-activating CRISPR RNA (tracrRNA-like) molecule.

In another system, System 77, the present disclosure provides the self-inactivating CRISPR-Cas system of any one of Systems 62-74, wherein the second target gRNA is a single RNA molecule.

In another system, System 78, the present disclosure provides the self-inactivating CRISPR-Cas system of any one of Systems 61-77, wherein the self-inactivating CRISPR-Cas system comprises a first vector comprising the first nucleic acid segment, and a second vector comprising the second nucleic acid segment.

In another system, System 79, the present disclosure provides the self-inactivating CRISPR-Cas system of any one of claims 61-77, wherein the self-inactivating CRISPR-Cas system comprises a first vector comprising the first and second nucleic acid segments.

In another system, System 80, the present disclosure provides the self-inactivating CRISPR-Cas system of Systems 78-79, wherein at least one of the first vector and the second vector is an adeno-associated virus (AAV) vector.

In another system, System 81, the present disclosure provides the self-inactivating CRISPR-Cas system of System 80, wherein the vector is AAV2.

In another system, System 82, the present disclosure provides the self-inactivating CRISPR-Cas system of any one of Systems 61-81, wherein the site-directed Cas9 polypeptide is Staphylococcus aureus Cas9 (SaCas9) or a variant thereof.

In another system, System 83, the present disclosure provides he self-inactivating CRISPR-Cas system of any one of Systems 61-82, wherein the site-directed Cas9 polypeptide comprises the amino acid sequence set forth in SEQ ID NO: 1, SEQ ID NO: 2, SEQ ID NO: 3 or SEQ ID NO: 4.

In another system, System 84, the present disclosure provides the self-inactivating CRISPR-Cas system of any one of Systems 61-83, wherein the nucleotide sequence that encodes the site-directed Cas9 polypeptide comprises SEQ ID NO: 79.

In another system, System 85, the present disclosure provides the self-inactivating CRISPR-Cas system of any one of Systems 61-84, wherein the PAM sequence in the SIN site is selected from the group consisting of: NNGRRT, NRG, NAAAAN, NAAAAC, NNNNGHTT, YTN, NNNNACA, NNNACAC, NNVRYAC, NNNVRYM, NNAAAAW, or NNAGAAW.

In another system, System 86, the present disclosure provides the self-inactivating CRISPR-Cas system of any one of Systems 61-85, wherein the nucleotide sequence of the SIN site within the nucleotide sequence encoding the Cas9 polypeptide or variant thereof comprises a nucleotide sequence selected from the group consisting of SEQ ID NO: 63-72.

In another system, System 87, the present disclosure provides the self-inactivating CRISPR-Cas system of any one of Systems 61-86, wherein the nucleotide sequence of the SIN site is less than 25 nucleotides in length.

In another system, System 88, the present disclosure provides the self-inactivating CRISPR-Cas system of any one of Systems 61-87, wherein the nucleotide sequence of the SIN site within the nucleotide sequence encoding the Cas9 polypeptide or variant thereof comprises a nucleotide sequence selected from the group consisting of SEQ ID NO: 64, SEQ ID NO: 66; SEQ ID NO: 67; SEQ ID NO: 69 and SEQ ID NO: 72.

In another system, System 89, the present disclosure provides the self-inactivating CRISPR-Cas system of System 88, wherein the nucleotide sequence of the SIN site comprises the nucleotide sequence set forth in SEQ ID NO: 64.

In another system, System 90, the present disclosure provides the self-inactivating CRISPR-Cas system of any one of Systems 61-89, further comprising a second SIN site within the nucleotide sequence encoding the Cas9 polypeptide or variant thereof as a result of codon optimization.

In another system, System 91, the present disclosure provides the self-inactivating CRISPR-Cas system of System 90, wherein the second SIN site comprises a nucleotide sequence selected from the group consisting of SEQ ID NOs: 63-72.

In another system, System 92, the present disclosure provides the self-inactivating CRISPR-Cas system of System 90 or 91, wherein the wherein the first universal SIN site comprises the nucleotide sequence of SEQ ID NO: 64, and the second universal SIN site comprises a nucleotide sequence selected from SEQ ID NOs: 65-72.

In another system, System 93, the present disclosure provides the self-inactivating CRISPR-Cas system of System 92, wherein the second universal SIN site comprises a nucleotide sequence selected from SEQ ID NO: 65, SEQ ID NO: 67, SEQ ID NO: 68, SEQ ID NO: 69 and SEQ ID NO: 72.

In another system, System 94, the present disclosure provides the self-inactivating CRISPR-Cas system of any one of Systems 61-93, wherein (a) the SIN site within the nucleotide sequence encoding the Cas9 polypeptide or variant thereof comprises the nucleotide sequence of SEQ ID NO: 64, and the DNA-targeting segment of the gRNA which is complementary to the SIN site comprises the nucleotide sequence of SEQ ID NO: 87; (b) the SIN site within the nucleotide sequence encoding the Cas9 polypeptide or variant thereof comprises the nucleotide sequence of SEQ ID NO: 66, and the DNA-targeting segment of the gRNA which is complementary to the SIN site comprises the nucleotide sequence of SEQ ID NO: 88; (c) the SIN site within the nucleotide sequence encoding the Cas9 polypeptide or variant thereof comprises the nucleotide sequence of SEQ ID NO: 67, and the DNA-targeting segment of the gRNA which is complementary to the SIN site comprises the nucleotide sequence of SEQ ID NO: 89; (d) the SIN site within the nucleotide sequence encoding the Cas9 polypeptide or variant thereof comprises the nucleotide sequence of SEQ ID NO: 69, and the DNA-targeting segment of the gRNA which is complementary to the SIN site comprises the nucleotide sequence of SEQ ID NO: 90; or (e) the SIN site within the nucleotide sequence encoding the Cas9 polypeptide or variant thereof comprises the nucleotide sequence of SEQ ID NO: 72, and the DNA-targeting segment of the gRNA which is complementary to the SIN site comprises the nucleotide sequence of SEQ ID NO: 91.

In another system, System 95, the present disclosure provides the self-inactivating CRISPR-Cas system of any one of Systems 61-93, wherein (a) the SIN site within the nucleotide sequence encoding the Cas9 polypeptide or variant thereof comprises the nucleotide sequence of SEQ ID NO: 64, and the gRNA which is complementary to the SIN site comprises the nucleotide sequence of SEQ ID NO: 74; (b) the SIN site within the nucleotide sequence encoding the Cas9 polypeptide or variant thereof comprises the nucleotide sequence of SEQ ID NO: 66, and the gRNA which is complementary to the SIN site comprises the nucleotide sequence of SEQ ID NO: 75; (c) the SIN site within the nucleotide sequence encoding the Cas9 polypeptide or variant thereof comprises the nucleotide sequence of SEQ ID NO: 67, and the gRNA which is complementary to the SIN site comprises the nucleotide sequence of SEQ ID NO: 76; (d) the SIN site within the nucleotide sequence encoding the Cas9 polypeptide or variant thereof comprises the nucleotide sequence of SEQ ID NO: 69, and the gRNA which is complementary to the SIN site comprises the nucleotide sequence of SEQ ID NO: 77; or (e) the SIN site within the nucleotide sequence encoding the Cas9 polypeptide or variant thereof comprises the nucleotide sequence of SEQ ID NO: 72, and the gRNA which is complementary to the SIN site comprises the nucleotide sequence of SEQ ID NO: 78.

In another system, System 96, the present disclosure provides a self-inactivating CRISPR-Cas system comprising: (a) a first nucleic acid segment comprising (i) a nucleotide sequence that encodes a site-directed Cas9 polypeptide or variant thereof, and (ii) a chimeric intron comprising a self-inactivating (SIN) site; and (b) a second nucleic acid segment comprising a nucleotide sequence that encodes a guide RNA (gRNA), wherein the gRNA comprises a DNA-targeting sequence that is complementary to a SIN site in the first nucleic acid segment.

In another system, System 97, the present disclosure provides the self-inactivating CRISPR-Cas system of Systems 95, wherein the chimeric intron is inserted into the Cas9 open reading frame (ORF).

In another system, System 98, the present disclosure provides the self-inactivating CRISPR-Cas system of Systems 97, wherein the chimeric intron is inserted before or after the codon encoding amino acid N580 of the Cas9 polypeptide or variant thereof.

In another system, System 99, the present disclosure provides the self-inactivating CRISPR-Cas system of System 97, wherein the chimeric intron is inserted before or after the codon encoding amino acid D10 of the Cas9 polypeptide or variant thereof.

In another system, System 100, the present disclosure provides the self-inactivating CRISPR-Cas system of any one of Systems 36-39, wherein the chimeric intron comprises a 5′-donor site from the first intron of the human β-globin gene and the branch and 3′-acceptor site from the intron of an immunoglobulin heavy chain variable region.

In another system, System 101, the present disclosure provides the self-inactivating CRISPR-Cas system of any one of Systems 36-40, wherein the SIN site in the chimeric intron is complementary to the DNA-targeting sequence of a first target gRNA that binds to a target gene in the cell.

In another system, System 102, the present disclosure provides the self-inactivating CRISPR-Cas system of any one of Systems 96-100, wherein the chimeric intron comprises a nucleotide sequence selected from the group consisting of SEQ ID NOs: 114, 115, 116, 118 or 120.

In another system, System 103, the present disclosure provides the self-inactivating CRISPR-Cas system of any one of Systems 99-102, wherein the first nucleic acid segment comprises a second SIN site.

In another system, System 104, the present disclosure provides the self-inactivating CRISPR-Cas system of System 43, wherein the second SIN site is located: (a) at the 5′ end of the nucleotide sequence encoding the Cas9 polypeptide or variant thereof; (b) at the 3′ end of the nucleotide sequence encoding the Cas9 polypeptide or variant thereof; (c) within the nucleotide sequence encoding the Cas9 polypeptide or variant thereof.

In another system, System 105, the present disclosure provides the self-inactivating CRISPR-Cas system of System 103 or 104, wherein the nucleotide sequence of the first and second SIN sites in the first nucleic acid segment are the same.

In another system, System 106, the present disclosure provides the self-inactivating CRISPR-Cas system of System 103 or 104, wherein the nucleotide sequence of the first and second target SIN sites in the first nucleic acid segment are different.

In another system, System 107, the present disclosure provides the self-inactivating CRISPR-Cas system of System 96, wherein the second SIN site is complementary to the DNA-targeting sequence of a second target gRNA that binds to the target gene in the cell.

In another system, System 108, the present disclosure provides the self-inactivating CRISPR-Cas system of System 97, wherein the second SIN site is located at the 5′ end of the nucleotide sequence encoding the Cas9 polypeptide or variant thereof.

In another system, System 109, the present disclosure provides the self-inactivating CRISPR-Cas system of System 97, wherein the second SIN site is located at the 3′ end of the nucleotide sequence encoding the Cas9 polypeptide or variant thereof.

In another system, System 110, the present disclosure provides the self-inactivating CRISPR-Cas system of any one of Systems 96-104, wherein the second SIN site is located within the nucleotide sequence encoding the Cas9 polypeptide or variant thereof.

In another system, System 111, the present disclosure provides the self-inactivating CRISPR-Cas system of System 110, wherein the second SIN site is within the open reading frame (ORF) of the nucleotide sequence encoding the Cas9 polypeptide or variant thereof.

In another system, System 112, the present disclosure provides the self-inactivating CRISPR-Cas system of any one of Systems 96-111, wherein the gRNA that is complementary to the SIN site in the chimeric intron is a two-molecule guide RNA.

In another system, System 113, the present disclosure provides the self-inactivating CRISPR-Cas system of System 102, wherein the two-molecule guide RNA comprises a CRISPR RNA (crRNA-like) molecule and a trans-activating CRISPR RNA (tracrRNA-like) molecule.

In another system, System 114, the present disclosure provides the self-inactivating CRISPR-Cas system of any one of Systems 96-111, wherein the gRNA that is complementary to the SIN site in the chimeric intron is a single RNA molecule.

In another system, System 115, the present disclosure provides the self-inactivating CRISPR-Cas system of any one of Systems 103-114, wherein the gRNA that is complementary to the second SIN site in the first nucleic acid segment is a two-molecule guide RNA.

In another system, System 116, the present disclosure provides the self-inactivating CRISPR-Cas system of System 115, wherein the two-molecule guide RNA that is complementary to the second SIN site comprises a CRISPR RNA (crRNA-like) molecule and a trans-activating CRISPR RNA (tracrRNA-like) molecule.

In another system, System 117, the present disclosure provides the self-inactivating CRISPR-Cas system of any one of Systems 103-114, wherein the gRNA that is complementary to the second SIN site in the first nucleic acid segment is a single RNA molecule.

In another system, System 118, the present disclosure provides the self-inactivating CRISPR-Cas system of any one of Systems 96-115, wherein the self-inactivating CRISPR-Cas system comprises a first vector comprising the first nucleic acid segment, and a second vector comprising the second nucleic acid segment.

In another system, System 119, the present disclosure provides the self-inactivating CRISPR-Cas system of any one of Systems 96-115, wherein the self-inactivating CRISPR-Cas system comprises a first vector comprising the first and second nucleic acid segments.

In another system, System 120, the present disclosure provides the self-inactivating CRISPR-Cas system of System 115, wherein the second vector comprises a second nucleotide sequence that encodes a second target gRNA that comprises a DNA-targeting sequence that is complementary to the target gene in a cell.

In another system, System 121, the present disclosure provides the self-inactivating CRISPR-Cas system of any one of Systems 118-120, wherein at least one of the first vector and the second vector is an adeno-associated virus (AAV) vector.

In another system, System 122, the present disclosure provides the self-inactivating CRISPR-Cas system of System 121, wherein at least one of the first vector and the second vector is AAV2.

In another system, System 123, the present disclosure provides the self-inactivating CRISPR-Cas system of any one of Systems 96-122, wherein the site-directed Cas9 polypeptide is Staphylococcus aureus Cas9 (SaCas9) or a variant thereof.

In another system, System 124, the present disclosure provides the self-inactivating CRISPR-Cas system of System 123, wherein the site-directed Cas9 polypeptide comprises the amino acid sequence set forth in SEQ ID NO: 1, SEQ ID NO: 2, SEQ ID NO: 3 or SEQ ID NO: 4.

In another system, System 125, the present disclosure provides the self-inactivating CRISPR-Cas system of any one of Systems 96-124, wherein the nucleotide sequence encoding the Cas9 polypeptide or variant thereof is codon optimized.

In another system, System 126, the present disclosure provides the self-inactivating CRISPR-Cas system of any one of Systems 36-62, wherein the nucleotide sequence that encodes the site-directed Cas9 polypeptide comprises SEQ ID NO: 79.

In a first genetically modified cell, Genetically Modified Cell 1, the present disclosure provides a genetically modified cell comprising the self-inactivating CRISPR-Cas system of any of Systems 1-36.

In another genetically modified cell, Genetically Modified Cell 2, the present disclosure provides the genetically modified cell of Genetically Modified Cell 1, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In another genetically modified cell, Genetically Modified Cell 3, the present disclosure provides a genetically modified cell comprising the self-inactivating CRISPR-Cas system of any of Systems 37-60.

In another genetically modified cell, Genetically Modified Cell 4, the present disclosure provides the genetically modified cell of Genetically Modified Cell 3, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In another genetically modified cell, Genetically Modified Cell 5, the present disclosure provides a genetically modified cell comprising the self-inactivating CRISPR-Cas system of any one of Systems 61-126.

In another genetically modified cell, Genetically Modified Cell 6, the present disclosure provides the genetically modified cell of Genetically Modified Cell 5, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In another genetically modified cell, Genetically Modified Cell 7, the present disclosure provides the genetically modified cell of Genetically Modified Cell 6, wherein the cell is a stem cell selected from the group consisting of an embryonic stem (ES) cell, and an induced pluripotent stem (iPS) cell.

In another genetically modified cell, Genetically Modified Cell 8, the present disclosure provides the genetically modified cell of Genetically Modified Cell 6, wherein the cell is a somatic cell selected from the group consisting of a fibroblast, a hematopoietic cell, a neuron, a muscle cell, a bone cell, a hepatocyte, and a pancreatic cell.

In a first method, Method 1, the present disclosure provides a method of controlling Cas9 expression in a cell comprising: contacting the cell with the self-inactivating CRISPR-Cas system of any one of Systems 1-36.

In another method, Method 2, the present disclosure provides a method of controlling Cas9 expression in a cell, as provided in Method 1, further comprising transforming the cell with a third vector comprising a nucleotide sequence encoding a homology-directed repair (HDR) template.

In another method, Method 3, the present disclosure provides a method of controlling Cas9 expression in a cell comprising: contacting the cell with the self-inactivating CRISPR-Cas system of any one of Systems 37-60.

In another method, Method 4, the present disclosure provides a method of controlling Cas9 expression in a cell, as provided in Method 3, further comprising contacting the cell with a third vector comprising a nucleotide sequence encoding a homology-directed repair (HDR) template.

In another method, Method 5, the present disclosure provides a method of genetically modifying a cell comprising the step of contacting the cell with the self-inactivating CRISPR-Cas system of any one of Systems 37-60.

In another method, Method 6, the present disclosure provides a method of genetically modifying a cell comprising the step of contacting the cell with the self-inactivating CRISPR-Cas system of any one of Systems 61-126.

In another method, Method 7, the present disclosure provides the method of Method 6, further comprising the step of contacting the cell with a third vector comprising a nucleotide sequence encoding a homology-directed repair (HDR) template.

In a first composition, Composition 1, the present disclosure provides a pharmaceutical composition comprising the self-inactivating CRISPR-Cas system of any of systems 1-36.

In another composition, Composition 2, the present disclosure provides pharmaceutical composition of Composition 1, wherein the composition is sterile.

In another composition, Composition 3, the present disclosure provides a pharmaceutical composition comprising the self-inactivating CRISPR-Cas system of any of systems 37-60.

In another composition, Composition 4, the present disclosure provides a pharmaceutical composition of Composition 3, wherein the composition is sterile.

In another composition, Composition 5, the present disclosure provides a nucleic acid for use in a self-inactivating CRISPR-Cas system comprising a codon optimized sequence encoding a site-directed polypeptide, wherein the codon optimized sequence further comprises a SIN site.

In another composition, Composition 6, the present disclosure provides a nucleic acid of Composition 5, wherein the SIN site comprises the PAM NNGRRT, or variant thereof.

In another composition, Composition 7, the present disclosure provides a nucleic acid of any of Compositions 5-6, wherein the SIN site comprises a sequence selected from the group consisting of SEQ ID NOs: 63 to 72.

In another composition, Composition 8, the present disclosure provides a nucleic acid of any of Compositions 5-6, wherein the codon optimized sequence comprises SEQ ID NO: 79.

In another composition, Composition 9, the present disclosure provides a nucleic acid for use in a self-inactivating CRISPR-Cas system comprising a codon optimized sequence encoding a site-directed polypeptide and one or more SIN sites, wherein the one or more SIN sites are located at any one or more of: a) a 5′ end of the first segment, upstream of the start codon and/or downstream of the transcriptional start site; b) within one or more naturally occurring or chimeric inserted introns; or c) a 3′ end of the first segment between the stop codon and poly(A) termination site.

In another composition, Composition 10, the present disclosure further provides a vector comprising the compositions of any one of Compositions 5-9.

In another composition, Composition 11, the present disclosure provides a pharmaceutical composition comprising the self-inactivating CRISPR-Cas system of any one of Systems 61-126.

In another composition, Composition 12, the present disclosure provides a pharmaceutical composition comprising the genetically modified cell of any one of Genetically Modified Cells 5-8.

In another composition, Composition 13, the present disclosure provides a nucleic acid for use in a self-inactivating CRISPR-Cas system comprising a codon optimized nucleotide sequence encoding a site-directed Cas9 polypeptide or variant thereof, wherein the codon optimized sequence comprises a self-inactivating (SIN) site and an adjacent Protospacer Adjacent Motif (PAM) within the open reading frame (ORF).

In another composition, Composition 14, the present disclosure provides the nucleic acid of Composition 13, wherein the SIN site comprises a sequence selected from the group consisting of SEQ ID NOs: 63 to 72.

In another composition, Composition 15, the present disclosure provides a nucleic acid for use in a self-inactivating CRISPR-Cas system comprising (i) a nucleotide sequence that encodes a site-directed Cas9 polypeptide or variant thereof, and (ii) a chimeric intron comprising a self-inactivating (SIN) site.

In another composition, Composition 16, the present disclosure provides the nucleic acid of Composition 15, wherein the open reading frame (ORF) of the nucleotide sequence that encodes a site-directed Cas9 polypeptide or variant thereof is codon optimized.

In another composition, Composition 17, the present disclosure provides the nucleic acid of Compoistion 15 or Composition 16, wherein the SIN site comprises a sequence selected from the group consisting of SEQ ID NOs: 63 to 72.

### Examples

The invention will be more fully understood by reference to the following examples, which provide illustrative non-limiting aspects of the invention. The examples herein describe use of a self-inactivating CRISPR system to modulate duration of SaCas9 protein expression while effectively retaining genomic modification potential. Use of defined target specific gRNAs to limit duration of Cas9 expression in-vivo represents a novel strategy to reduce immune/inflammatory responses to Cas9 protein and also minimize/eliminate any potential off-target effects of Cas9 which can translate to enhanced safety and efficacy of CRISPR-Cas system for in vivo gene editing as described and illustrated herein.

### Example 1—Testing of SaCas9 Protein Expression

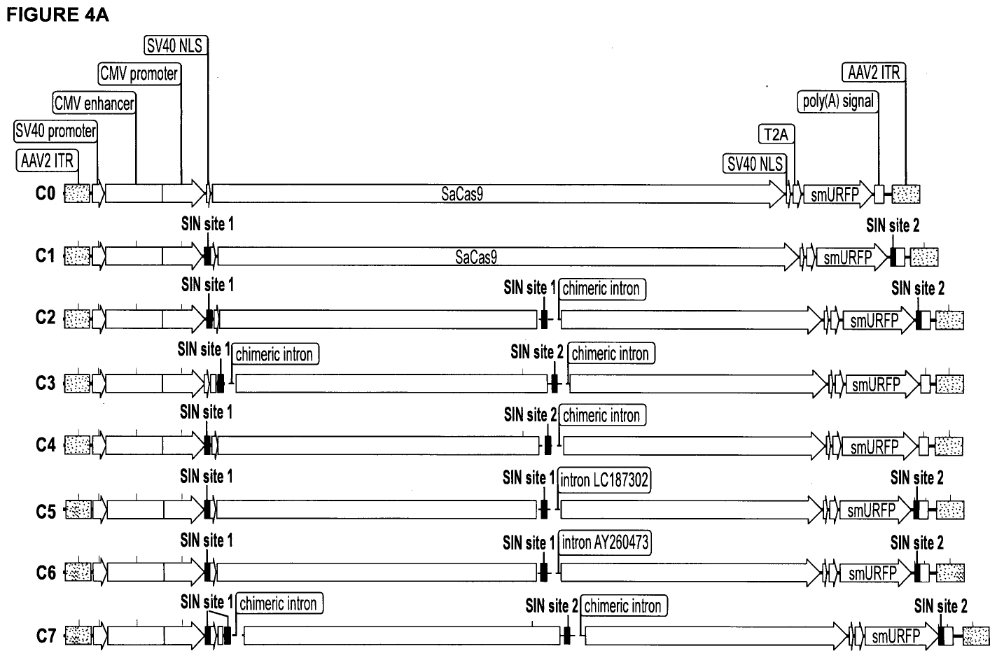

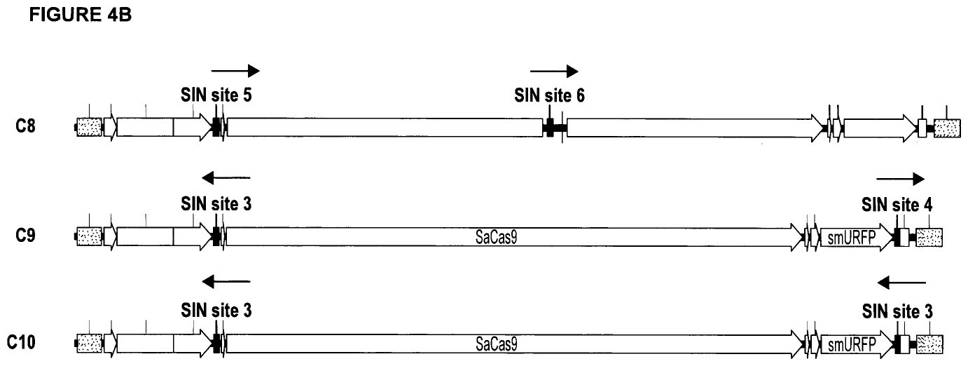

Selected spacer sequences and their corresponding PAM sequences (SIN sites) were cloned into various locations of a SaCas9 expression cassette. The number of SIN sites cloned into the SaCas9 expression cassette varied between 2-4 SIN sites per SaCas9 expression cassette (See Table 4). As illustrated in FIGS. 4A-B, SIN sites were introduced (a) at the 5′ end, upstream of the start codon and/or downstream of the transcriptional start site of SaCas9, (b) within one or more naturally occurring or chimeric introns cloned at various locations of SaCas9 ORF, and (c) at the 3′ end between the stop codon and poly(A) termination site.

TABLE 4
SIN Site Sequences for Constructs C0-C7

| | | #of | | |
|---|---|---|---|---|
| | Construct | SIN | | |
| Construct | SEQ ID NO. | sites | SIN site 1 | SIN site 2 |
| C0 | 92 | 0 | — | — |
| C1 | 93 | 2 | GTGTATTGCTTGTACTACTC | GTGTTATTACTTGCTACTGCA |
| | | | ACTGAAT (SEQ ID NO: 16) | GAGAGT (SEQ ID NO: 17) |
| C2 | 94 | 3 | GTGTATTGCTTGTACTACTC | GTGTTATTACTTGCTACTGCA |
| | | | ACTGAAT (SEQ ID NO: 16) | GAGAGT (SEQ ID NO: 17) |
| C3 | 95 | 2 | GTGTATTGCTTGTACTACTC | GTGTTATTACTTGCTACTGCA |
| | | | ACTGAAT (SEQ ID NO: 16) | GAGAGT (SEQ ID NO: 17) |
| C4 | 96 | 2 | GTGTATTGCTTGTACTACTC | GTGTTATTACTTGCTACTGCA |
| | | | ACTGAAT (SEQ ID NO: 16) | GAGAGT (SEQ ID NO: 17) |
| C5 | 97 | 2 | GTGTATTGCTTGTACTACTC | GTGTTATTACTTGCTACTGCA |
| | | | ACTGAAT (SEQ ID NO: 16) | GAGAGT (SEQ ID NO: 17) |
| C6 | 98 | 2 | GTGTATTGCTTGTACTACTC | GTGTTATTACTTGCTACTGCA |
| | | | ACTGAAT (SEQ ID NO: 16) | GAGAGT (SEQ ID NO: 17) |
| C7 | 99 | 4 | GTGTATTGCTTGTACTACTC | GTGTTATTACTTGCTACTGCA |
| | | | ACTGAAT (SEQ ID NO: 16) | GAGAGT (SEQ ID NO: 17) |

Design and generation of plasmid/vectors. AAV vector plasmid constructs used in these Examples were built using standard cloning procedures and Gibson High-Fidelity assembly reactions based on manufacture's recommendations (New England Biolabs, Ipswich, Mass.). The vector plasmid constructs can be constructed using component sequences shown in Table 5.

TABLE 5
Component sequence for generating AAV vector constructs

| | | SEQ ID |
|---|---|---|
| Component | Sequence | NO: |
| 5′ AAV ITR | CCTGCAGGCAGCTGCGCGCTCGCTCGCTCACTGAGGCCGCC | 104 |
| | CGGGCGTCGGGCGACCTTTGGTCGCCCGGCCTCAGTGAGCG | |
| | AGCGAGCGCGCAGAGAGGGAGTGGCCAACTCCATCACTAG | |
| | GGGTTCCT | |
| SV40 Promoter | GGTGTGGAAAGTCCCCAGGCTCCCCAGCAGGCAGAAGTATG | 105 |
| | CAAAGCATGCATCTCAATTAGTCAGCAACCA | |
| CMV enhancer | CGTTACATAACTTACGGTAAATGGCCCGCCTGGCTGACCGC | 106 |
| | CCAACGACCCCCGCCCATTGACGTCAATAATGACGTATGTT | |
| | CCCATAGTAACGCCAATAGGGACTTTCCATTGACGTCAATG | |
| | GGTGGAGTATTTACGGTAAACTGCCCACTTGGCAGTACATC | |
| | AAGTGTATCATATGCCAAGTACGCCCCCTATTGACGTCAAT | |
| | GACGGTAAATGGCCCGCCTGGCATTATGCCCAGTACATGAC | |
| | CTTATGGGACTTTCCTACTTGGCAGTACATCTACGTATTAGT | |
| | CATCGCTATTACCATG | |
| CMV promoter | GTGATGCGGTTTTGGCAGTACATCAATGGGCGTGGATAGCG | 107 |
| | GTTTGACTCACGGGGATTTCCAAGTCTCCACCCCATTGACGT | |
| | CAATGGGAGTTTGTTTTGGCACCAAAATCAACGGGACTTTC | |
| | CAAAATGTCGTAACAACTCCGCCCCATTGACGCAAATGGGC | |
| | GGTAGGCGTGTACGGTGGGAGGTCTATATAAGCAGAGCTCG | |
| | TTTAGTGAACCGT | |
| SV40 NLS | ATGGCCCCAAAGAAGAAGCGGAAGGTC | 108 |
| SaCas9 | GGAAAGCGGAACTATATCCTGGGACTGGACATCGGAATTAC | 79 |
| | CTCCGTGGGATACGGCATCATCGATTACGAGACTAGGGACG | |
| | TGATTGACGCCGGCGTGAGACTCTTTAAGGAGGCCAACGTG | |
| | GAAAACAACGAAGGTCGCAGATCCAAGCGGGGTGCAAGAC | |
| | GCCTGAAGCGCCGGAGGAGACATCGGATACAGCGCGTGAA | |
| | GAAGCTCCTTTTCGACTACAACCTCCTCACTGACCACTCGGA | |
| | ATTGTCCGGTATCAACCCCTACGAAGCCCGCGTGAAAGGCC | |
| | TGAGCCAGAAGCTGTCCGAAGAGGAGTTTAGCGCAGCCCTG | |
| | CTGCACCTGGCTAAGCGAAGGGGGGTGCACAACGTGAACG | |
| | AGGTGGAGGAGGACACTGGCAACGAACTGTCCACCAAGGA | |
| | GCAGATTTCACGGAACTCGAAGGCGCTGGAAGAGAAATATG | |
| | TGGCCGAGCTGCAGCTGGAGAGGCTCAAGAAGGATGGCGA | |
| | AGTCCGGGGGAGCATCAATCGCTTCAAGACCTCGGACTACG | |
| | TGAAGGAAGCCAAACAGCTGTTGAAGGTGCAGAAGGCCTA | |
| | CCACCAACTGGACCAATCATTCATTGACACTTACATCGATCT | |
| | GCTTGAAACCAGGCGCACCTACTACGAGGGTCCTGGAGAAG | |
| | GCAGCCCTTTCGGATGGAAGGACATCAAGGAGTGGTATGAG | |
| | ATGCTGATGGGTCATTGCACCTACTTTCCGGAAGAACTGCG | |
| | CTCAGTGAAGTACGCGTACAACGCTGACCTCTACAACGCTC | |
| | TCAACGATCTGAACAACCTCGTGATCACCCGGGACGAGAAC | |
| | GAAAAGCTGGAGTACTACGAAAAGTTCCAGATTATCGAAAA | |
| | CGTGTTCAAGCAGAAGAAGAAGCCCACCCTGAAGCAGATTG | |
| | CAAAGGAGATCCTTGTGAACGAGGAGGATATTAAGGGCTAC | |
| | CGGGTCACCTCCACCGGGAAACCAGAGTTCACTAATCTCAA | |
| | GGTGTACCATGACATTAAGGACATTACTGCCCGCAAGGAGA | |
| | TCATTGAAAACGCGGAACTGCTGGACCAAATCGCGAAGATC | |
| | CTGACCATCTATCAGAGCTCCGAGGATATCCAGGAGGAACT | |
| | TACTAACCTCAATTCCGAGCTGACGCAGGAAGAAATCGAGC | |
| | AAATTAGCAACCTGAAGGGTTACACTGGAACCCACAACCTC | |
| | AGCTTGAAAGCGATTAACCTTATTTTGGATGAACTTTGGCAC | |
| | ACTAATGACAATCAGATCGCCATTTTCAACCGGCTGAAACT | |
| | GGTGCCGAAGAAGGTGGACCTGAGCCAACAGAAGGAAATC | |
| | CCGACCACCCTTGTGGACGATTTCATCCTGTCACCTGTGGTG | |
| | AAGAGGAGCTTCATCCAGTCGATCAAGGTCATCAACGCCAT | |
| | CATAAAGAAGTACGGCCTTCCCAACGACATCATCATCGAAC | |
| | TGGCCCGCGAGAAGAACTCCAAAGATGCCCAGAAGATGATC | |
| | AACGAGATGCAGAAGCGAAACCGGCAGACGAACGAACGGA | |
| | TCGAGGAGATCATCCGGACCACCGGGAAGGAAAACGCGAA | |
| | GTACCTGATCGAGAAAATCAAGCTGCATGATATGCAGGAAG | |
| | GGAAGTGTCTCTACTCCCTGGAGGCCATTCCGCTGGAGGAT | |
| | TTGCTGAACAACCCTTTCAACTACGAAGTCGATCATATCATT | |
| | CCTCGCTCCGTGTCCTTCGATAACTCCTTCAACAATAAGGTC | |
| | CTCGTGAAGCAGGAGGAGAACTCGAAGAAGGGCAACAGAA | |
| | CCCCGTTCCAGTACCTCTCGTCGTCCGACTCCAAGATCAGCT | |
| | ACGAAACTTTCAAGAAGCACATTCTGAACCTGGCCAAGGGC | |
| | AAAGGGAGAATTAGCAAGACCAAGAAGGAATACCTCCTGG | |
| | AAGAGAGAGACATCAACCGCTTCTCGGTGCAAAAGGATTTC | |
| | ATCAACCGCAACCTGGTCGATACCAGATACGCCACCAGGGG | |
| | ACTGATGAACCTCCTGCGGTCCTACTTCCGGGTCAACAATCT | |
| | GGACGTGAAGGTCAAATCCATCAACGGGGGCTTTACTTCTT | |
| | TCCTGCGCCGGAAGTGGAAGTTCAAGAAGGAACGGAACAA | |
| | GGGATACAAGCACCACGCTGAAGATGCCCTGATTATTGCCA | |
| | ACGCCGACTTCATCTTTAAGGAATGGAAAAAGCTGGACAAG | |
| | GCTAAGAAGGTCATGGAGAACCAGATGTTCGAAGAAAAGC | |
| | AGGCCGAGTCCATGCCCGAAATCGAAACCGAGCAGGAATA | |
| | CAAGGAGATCTTCATCACACCGCACCAAATCAAGCACATCA | |
| | AGGACTTCAAGGATTACAAGTACAGCCACCGGGTGGACAAG | |
| | AAGCCTAACAGAGAGCTTATCAACGACACCCTGTACTCCAC | |
| | GCGCAAGGACGACAAGGGAAACACATTGATCGTGAACAAC | |
| | CTGAACGGACTGTATGACAAGGACAATGACAAACTGAAGA | |
| | AGCTGATCAACAAATCGCCGGAAAAGCTCCTGATGTACCAT | |
| | CACGACCCTCAAACCTACCAGAAACTGAAGCTCATCATGGA | |
| | GCAGTACGGCGACGAAAAGAATCCCCTGTACAAATACTACG | |
| | AGGAGACTGGAAATTACCTGACTAAGTACTCCAAGAAGGAT | |
| | AACGGCCCCGTGATCAAGAAGATTAAGTACTACGGAAACAA | |
| | ACTGAACGCACATCTCGACATCACCGATGATTATCCAAACT | |
| | CCCGCAACAAAGTCGTGAAGCTCTCCCTCAAACCGTACCGC | |
| | TTCGACGTGTACCTGGATAATGGGGTGTACAAGTTCGTGAC | |
| | CGTGAAGAACCTGGACGTCATTAAGAAGGAAAACTACTACG | |
| | AAGTGAACTCAAAGTGCTACGAGGAAGCCAAGAAGCTCAA | |
| | GAAGATCAGCAACCAGGCCGAGTTCATCGCATCGTTTTACA | |
| | ACAATGACCTCATTAAGATTAATGGAGAACTGTACAGAGTG | |
| | ATCGGCGTGAACAACGACCTCCTGAACCGGATTGAAGTGAA | |
| | CATGATCGATATTACCTACCGGGAGTATCTGGAGAACATGA | |
| | ACGACAAGCGCCCACCGAGAATCATCAAAACTATTGCCTCC | |
| | AAGACCCAATCCATTAAGAAATACTCCACCGACATCCTGGG | |
| | CAACCTGTACGAGGTCAAGTCGAAGAAGCACCCCCAGATTA | |
| | TCAAGAAGGGA | |
| T2A promoter | GAGGGCAGGGGAAGTCTGCTAACATGCGGGGACGTGGAGG | 109 |
| | AAAATCCC | |
| smURFP | ATGGCTAAGACTTCCGAACAGAGGGTGAACATTGCTACACT | 110 |
| reporter gene | GCTGACAGAAAATAAGAAGAAAATCGTGGATAAGGCTTCCC | |
| cassette | AGGATCTGTGGCGGAGACACCCAGACCTGATCGCACCAGGA | |
| | GGAATTGCTTTCTCTCAGAGGGACCGCGCTCTGTGCCTGCGA | |
| | GATTACGGCTGGTTCCTGCATCTGATCACCTTTTGTCTGCTG | |
| | GCCGGAGATAAGGGCCCCATCGAGTCTATTGGGCTGATCAG | |
| | TATTCGAGAAATGTATAACTCACTGGGAGTGCCCGTCCCTG | |
| | CAATGATGGAGAGCATTAGATGCCTGAAAGAAGCCAGCCTG | |
| | TCCCTGCTGGACGAAGAGGACGCCAACGAGACCGCACCCTA | |
| | CTTTGATTACATTATTAAGGCTATGAGCTAA | |
| poly-A-site | AATAAAATATCTTTATTTTCATTACATCTGTGTGTTGGTTTTT | 111 |
| | TGTGTG | |
| 3′ AAV ITR | AGGAACCCCTAGTGATGGAGTTGGCCACTCCCTCTCTGCGC | 112 |
| | GCTCGCTCGCTCACTGAGGCCGGGCGACCAAAGGTCGCCCG | |
| | ACGCCCGGGCTTTGCCCGGGCGGCCTCAGTGAGCGAGCGAG | |
| | CGCGCAGCTGCCTGCAGG | |
| Chimeric intron | GTAAGTATCAAGGTTACAAGACAGGT [symbol] | 113 |
| | [symbol] CTTGTCGAGACAGAGAAGACTCTTGCGTTTCT | |
| | GATAGGCACCTATTGGTCTTACTGACATCCACTTTGCCTTTC | |
| | TCTCCACAG | |
| Chimeric intron | GTAAGTATCAAGGTTACAAGACAGGTGTATTGCTTGTACTA | 114 |
| with SIN site 1 | CTCACTGAATCTTGTCGAGACAGAGAAGACTCTTGCGTTTCT | |
| | GATAGGCACCTATTGGTCTTACTGACATCCACTTTGCCTTTC | |
| | TCTCCACAG | |
| Chimeric intron | GTAAGTATCAAGGTTACAAGACAGGTGTTATTACTTGCTACT | 115 |
| with SIN site 2 | GCAGAGAGTCTTGTCGAGACAGAGAAGACTCTTGCGTTTCT | |
| | GATAGGCACCTATTGGTCTTACTGACATCCACTTTGCCTTTC | |
| | TCTCCACAG | |
| Chimeric intron | GTAAGTATCAAGGTTACAAGACAGGTN₂₀- | : 116 |
| with SIN site | N₃₅CTTGTCGAGACAGAGAAGACTCTTGCGTTTCTGATAGGC | |
| | ACCTATTGGTCTTACTGACATCCACTTTGCCTTTCTCTCCACA | |
| | G | |
| BCL11A intron | GTATGTCTACATTTCTCTTAGGTAAACATCTAAGGCATTTCG | 117 |
| 2-genbank ID | AGAACACAGAAAAGGTTTTGAGTTTGAG | |
| LC187302.1 | | |
| Intron | GTATGTGTATTGCTTGTACTACTCACTGAATCTACATTTCTCT | 118 |
| LC187302 with | TAGGTAAACATCTAAGGCATTTCGAGAACACAGAAAAGGTT | |
| SIN site 1 | TTGAGTTTGAG | |
| Retinoblastoma | GTTAATATTTCATAAATAGTTACTTTTTTTTTCATTTTTAGGA | 119 |
| intron 16- | AG | |
| genbank ID | | |
| AY260473.1 | | |
| Intron | GTTAATATTTCATAGTGTATTGCTTGTACTACTCACTGAATA | 120 |
| AY260473 | GTTACTTTTTTTTTCATTTTTAG | |
| with SIN site 1 | | |

*underline sequence in Table 5 marks the SIN site. SIN sites can be inserted into the intron sequence with or without deletions in the intron. Sequence in bold italics indicates intron sequence that can be deleted and/or replace by the SIN site. (SEQ ID No: 116)

Various linkers known in the art may be used, for example: GGCCCC, GGTACTAGT, or AAGCTT, as well as others. Reporter genes such as the smURFP reporter gene cassette may be included.

The resulting constructs C0-C7 were transfected into HEK293T or myogenic cells to examine kinetics of protein expression by Immunoassay (FIGS. 5A-B).

Human Embryonic Kidney (HEK293T) cells (from ATCC, Manassas, Va.) or Myogenic cells (Cook Myosite, Pittsburgh, Pa.) were cultured and maintained at a low passage number as per the manufacture's recommendation. In preparation for transfection, HEK293T cells were added to 12-well plates at 400,000 cells/well and transfected 12-24 hours later using Jetprime reagent kit (VWR, Radnor, Pa.). For electroporation of myogenic cells, 200,000 cells were mixed with 5 μg of plasmids in Solution P1 and electroporated into cells using 4D Nucleofector DS150 Program. Prior to cell harvest, protein expression was analyzed using Evos fluorescence microscope.

To determine Cas9 protein expression, cell pellets were treated with chilled RIPA buffer (Fisher Scientific, Waltham, Mass.) containing Protease Inhibitors (Sigma Aldrich, St. Louis, Mo.) and incubated at 4° C. for 30 minutes. Cell debris was cleared using high-speed spin at 10,000×g for 10 mins at 4° C. Protein samples were loaded onto Wes 12-230 kD capillary system (Protein Simple, San Jose, Calif.). SaCas9 (EPR19799) and β-actin (RM112) protein antibodies were purchased (Abcam, Cambridge, Mass.). TurboGFP protein antibody was purchased (Fisher Scientific, Waltham, Mass.).

As shown in FIG. 5A, the introduction of a chimeric introns (located at either N580 or D10) in C2, C3, C4 and C7 did not affect the expression levels of SaCas9 in transfected cells. In each case, full-length saCas9 protein was expressed. The amounts of SaCas9 protein expressed was quantified relative to β-actin. In contrast, full-length Cas9 protein was not expressed for the C5 construct containing BCL11A intron 2 (LC187302) or the C6 construct containing retinoblastoma intron 16 (AY260473). Truncated SaCas9 protein was observed in these cells as illustrated in FIG. 5A. The truncated protein production with these two constructs was hypothesized to be due to failure of splicing.

### Example 2—Testing of Functionality of sgRNA SIN Sites on Cas9 Constructs

To examine the functionality of SIN sites in cleaving the SaCas9 constructs, linearized plasmids were incubated with ribonucleoprotein complexes (RNP) containing purified SaCas9 protein (SEQ ID NO: 1) and gRNA (where the gRNA spacer is complementary to a portion of the SIN site).

Purified plasmids were linearized with PsiI enzyme (New England Biolabs) and purified using ZymoClean DNA gel extraction kit (Zymo Research, Irvine, Calif.). Purified SaCas9 protein was purchased (Aldevron, Madison, Wis.). sgRNAs were expressed and purified using manufacture's recommended protocols (GeneArt Precision gRNA synthesis Kit, Life Technologies, Grand Island, N.Y.). For DNA digestion assay, SaCas9, sgRNA, and plasmid substrates were mixed in ratio of 10:10:1 and incubated for 2 hours at 37° C. DNA digestion patterns were analyzed using Flash-gel electrophoresis. The resulting products were analyzed by agarose gel electrophoresis.

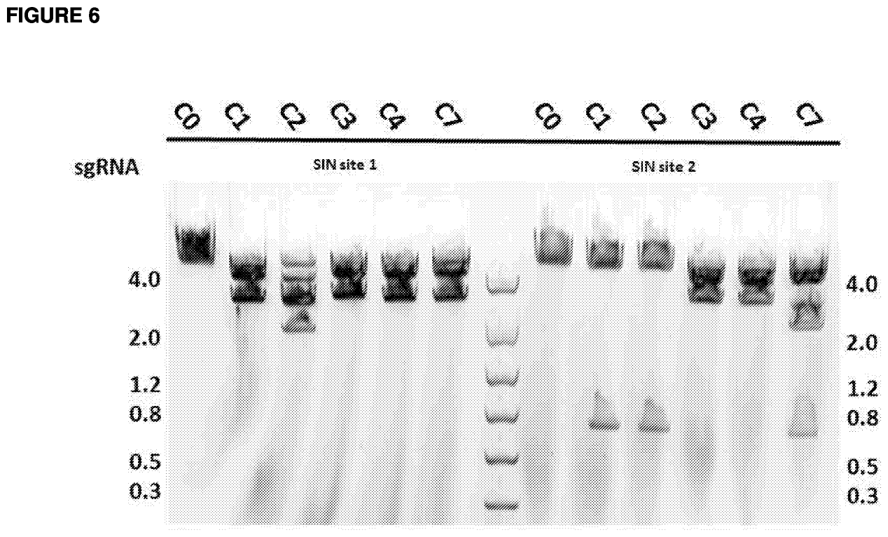

As shown in FIG. 6, the linearized C0 (control, with no SIN sites) in the DNA digestion assay resulted in one single DNA band, while the C1, C2, C3, C4 and C7 linearized DNA digestion assay samples resulted in more than one DNA fragment. The number of DNA fragments was dependent on the number of SIN sites. These results confirmed that incorporation of the SIN sites leads to cleavage of the construct, and the location of the SIN sites did not affect the ability of the RNP to cleave the construct.

### Example 3—Self-Inactivation (SIN) Kinetics of SaCas9 Constructs

FIG. 7A depicts the schematics of various plasmid constructs encoding gRNAs used to target the SIN sites in the Cas9 plasmid constructs (C2 to C7). The gRNA constructs used in this Example were generated in two forms: a or b (e.g.: G1a or G1b). The difference between the a and b constructs is the sequence used for the gRNA backbone. ‘a’ constructs express a gRNA backbone comprising SEQ ID NOs: 5 or 59 while the ‘b’ construct express a gRNA backbone comprising SEQ ID NOs: 6 or 60.

TABLE 6
sgRNAs expressed from gRNA expression constructs

| Construct | sgRNA | sgRNA |
|---|---|---|
| G1a, G2a, | sgRNA 1 | sgRNA 2 |
| or G3a | (SEQ ID NO: 22) | (SEQ ID NO: 23) |
| G1b, G2b, | sgRNA 1 | sgRNA 2 |
| or G3b | (SEQ ID NO: 61) | (SEQ ID NO: 62) |

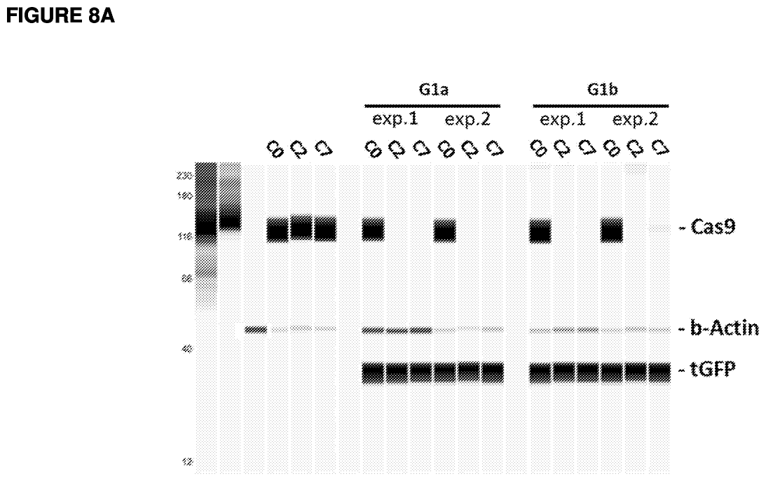

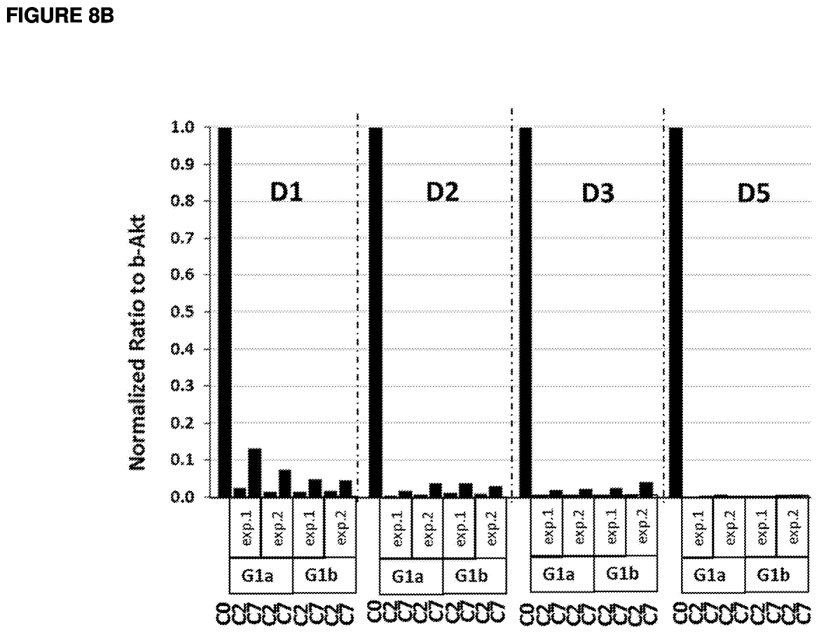

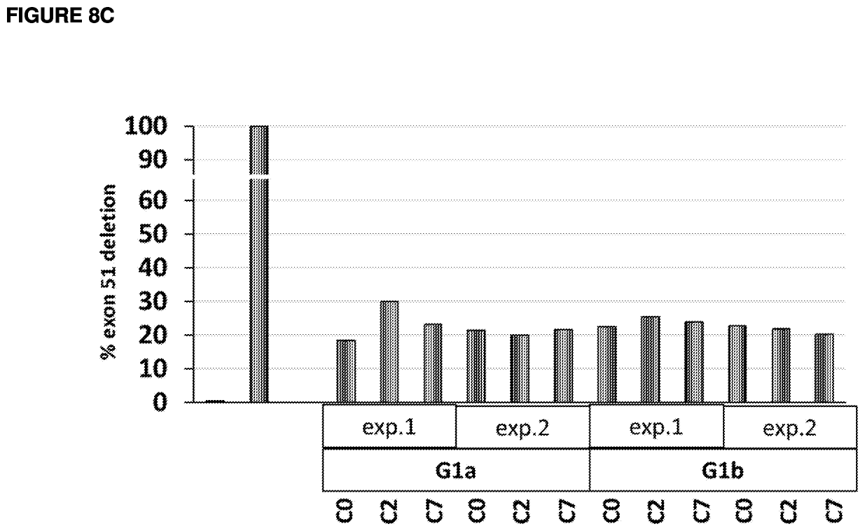

To test the ability of the gRNA constructs to express gRNA and further cleave the Cas9 construct at the SIN sites, Cas9 expressing plasmids containing SIN sites (FIG. 4A: C2 or C7) or Cas9 expressing plasmid constructs without SIN sites (FIG. 4A: C0) were transfected alone or co-transfected with plasmids encoding gRNAs (G1a or G1b) into HEK293T cells. Each plasmid construct (G1a or G1b) expressed two sgRNAs, sgRNA1 and sgRNA2. sgRNA1 comprises the spacer sequence (GUGUAUUGCUUGUACUACUCA; SEQ ID NO: 80) and targets SIN site 1. sgRNA2 comprises the spacer sequence (GUGUUAUUACUUGCUACUGCA; SEQ ID NO: 81) and targets SIN site 2. The transfected HEK293T cells were harvested post-transfection and the cell lysates monitored for Cas9 expression by Simple Wes analyses. Methods used in this Example were previously described in Example 1. FIG. 8A demonstrates that plasmids encoding gRNA targeting the SIN sites in the Cas9 expression vector can inhibit Cas9 expression. The reduction in Cas9 protein levels was observed within 24 hours as shown in FIG. 8B. The data demonstrates that the reduction in Cas9 protein levels is a function of gRNA activity since no reduction in Cas9 protein was observed when the Cas9 constructs were transfected alone or in a construct (C0) that did not contain a SIN site. The data shows that the temporal control of Cas9 expression is achieved by co-delivery of self-limiting Cas9 expressing constructs (e.g.: expression plasmids containing SIN sites) and plasmids encoding gRNAs that target the SIN site.

### Example 4—CRISPR/Cas9 Target Sites for the DMD Gene

Boundaries of exon 51 of the DMD gene were scanned for a protospacer adjacent motif (PAM) sequence, NNGRRT and spacer sequences were identified (20 bp and 21 bp spacer sequences). The identified gRNA spacer sequences are shown in Table 7. The SEQ ID NOs represent the DNA sequence of the genomic target, while the gRNA or sgRNA spacer sequence will be the RNA version of the DNA sequence. As described in the examples above, the self-inactivating AAV construct can be engineered with a guide RNA spacer sequence and PAM sequence, to create a SIN site. In some examples, the SIN site comprises a sequence that is also present in the target gene in the cell.

TABLE 7

| Left | Spacer Sequence | Right | Spacer Sequence |
|---|---|---|---|
| L02 | ACAATAAGTCAAATTTAATTG | R15 | AAATTGGCACAGACAACTTAG |
| | (SEQ ID NO: 34) | | (SEQ ID NO: 42) |
| L03 | AAGATATATAATGTCATGAAT | R22 | AAAAACAAGAAGTGAGGCAGA |
| | (SEQ ID NO: 35) | | (SEQ ID NO: 43) |
| L22 | GTGTATTGCTTGTACTACTCA | R42 | GTGTTATTACTTGCTACTGCA |
| | (SEQ ID NO: 36) | | (SEQ ID NO: 44) |
| L34 | TCTCCTCATTAGAGAAGAAG | R52 | ACACTTCCTTGTGACGGGTTT |
| | (SEQ ID NO: 37) | | (SEQ ID NO: 45) |
| L37 | CTCAAGCTTCTCAGGGACACC | R32 | CTATTCTGAGTACAGAGCATA |
| | (SEQ ID NO: 38) | | (SEQ ID NO: 46) |
| L61 | TCTTGCATCTTGCACATGTCC | | |
| | (SEQ ID NO: 39) | | |
| L64 | CTTAGAGGTCTTCTACATACA | | |
| | (SEQ ID NO: 40) | | |
| L81 | TTCTGACTGTAAGTACACTAT | | |
| | (SEQ ID NO: 41) | | |
| Left | Spacer Sequence | Right | Spacer Sequence |
| L02b | CAATAAGTCAAATTTAATTG | R15b | AATTGGCACAGACAACTTAG |
| | (SEQ ID NO: 47) | | (SEQ ID NO: 54) |
| L03b | AGATATATAATGTCATGAAT | R22b | AAAACAAGAAGTGAGGCAGA |
| | (SEQ ID NO: 48) | | (SEQ ID NO: 55) |
| L22b | TGTATTGCTTGTACTACTCA | R42b | TGTTATTACTTGCTACTGCA |
| | (SEQ ID NO: 49) | | (SEQ ID NO: 56) |
| L37b | TCAAGCTTCTCAGGGACACC | R52b | CACTTCCTTGTGACGGGTTT |
| | (SEQ ID NO: 50) | | (SEQ ID NO: 57) |
| L61b | CTTGCATCTTGCACATGTCC | R32b | TATTCTGAGTACAGAGCATA |
| | (SEQ ID NO: 51) | | (SEQ ID NO: 58) |
| L64b | TTAGAGGTCTTCTACATACA | | |
| | (SEQ ID NO: 52) | | |
| L81b | TCTGACTGTAAGTACACTAT | | |
| | (SEQ ID NO: 53) | | |

### Example 5—Self-Inactivation (SIN) Kinetics of SaCas9 Constructs Using Target Sites from the Human and Mouse DMD Genes

Additional gRNA and self-inactivating Cas9 constructs were designed to test additional self-inactivation sites using sequences from the human and murine dystrophin gene sequences (FIG. 7B). The ability of these gRNA constructs to express gRNA and cleave the corresponding Cas9 construct at the SIN sites was tested using the methods described above.

AAV vector plasmid constructs used in these Examples were built using standard cloning procedures and Gibson High-Fidelity assembly reactions based on manufacture's recommendations (New England Biolabs, Ipswich, Mass.). The C9 and C10 SaCas9 plasmid constructs contain SIN site sequences that correspond to target sites in the human dystrophin locus (FIG. 4B and Table 8). The SIN sites in C9 and C10 are present in the same relative orientation as the protospacer and PAM sequence found in the human genome. Thus, the sequence of SIN site 3 appears in the C9 and C10 construct sequence as the reverse complement sequence. The C8 plasmid constructs contains SIN site sequences that correspond to target sites in the murine dystrophin locus flanking exon 23 (FIG. 4B and Table 9).

FIG. 7B depict the schematics of plasmid constructs encoding the gRNAs used to target the SIN sites in the Cas9 plasmid constructs in this Example. Each gRNA construct was generated using a construct that expresses the gRNA backbone (“b”) comprising SEQ ID NOs: 6 or 60. Table 9 provides the sgRNA sequences expressed from the G4 and G5 plasmids. sgRNA3 comprises the spacer sequence CUUAGAGGUCUUCUACAUACA (SEQ ID NO: 82) and targets SIN site 3. sgRNA4 comprises the spacer sequence CUAUUCUGAGUACAGAGCAUA (SEQ ID NO: 83) and targets SIN site 4. sgRNA5 comprises the spacer sequence ACUAUGAUUAAAUGCUUGAUA (SEQ ID NO: 84) and targets SIN site 5. sgRNA6 comprises the spacer sequence CUUAAAGGCUUCAUAUAAGGG (SEQ ID NO: 85) and targets SIN site 6.

TABLE 8
SIN site Sequences for Constructs C9-C10

| | Construct | # of | | |
|---|---|---|---|---|
| | SEQ ID | SIN | | |
| Construct | NO: | sites | SIN site 3 | SIN site 4 |
| C9 | 101 | 2 | CTTAGAGGTCTTCTAC | CTATTCTGAGTACAGA |
| | | | ATACAATGAGT | GCATACAGAGT |
| | | | (SEQ ID NO: 18) | (SEQ ID NO: 19) |
| C10 | 102 | 2 | CTTAGAGGTCTTCTAC | — |
| | | | ATACAATGAGT | |
| | | | (SEQ ID NO: 18) | |

TABLE 9
SIN site Sequences for Construct C8

| | Construct | # of | | |
|---|---|---|---|---|
| | SEQ ID | SIN | | |
| Construct | NO: | sites | SIN site 5 | SIN site 6 |
| C8 | 100 | 2 | ACTATGATTAAATGC | CTTAAAGGCTTCAT |
| | | | TTGATATTGAGT | ATAAGGGTGGAAT |
| | | | (SEQ ID NO: 20) | (SEQ ID NO: 21) |

TABLE 10
sgRNAs expressed from gRNA expression constructs

| Construct | sgRNA | sgRNA |
|---|---|---|
| G4 | sgRNA 3 | sgRNA 4 |
| | (SEQ ID NO: 24) | (SEQ ID NO: 25) |
| G5 | sgRNA 5 | sgRNA 6 |
| | (SEQ ID NO: 26) | (SEQ ID NO: 27) |

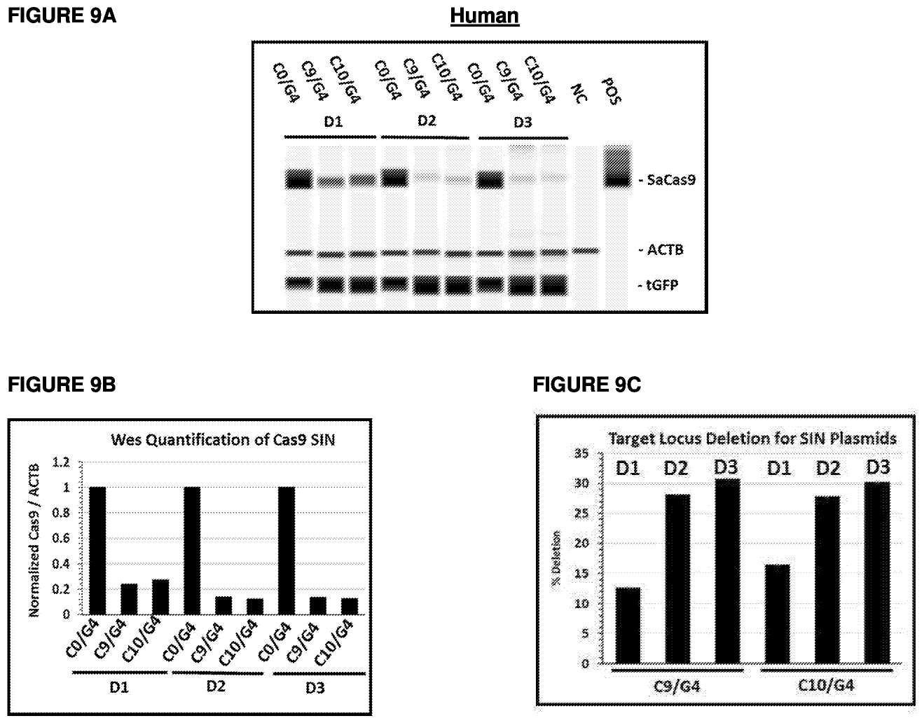

Cas9 expressing plasmids containing SIN sites (FIG. 4B: C9 or C10) or Cas9 expressing plasmid constructs without SIN sites (FIG. 4A: C0) were transfected alone or co-transfected with plasmids encoding gRNAs (G4) into HEK293T cells. The transfected HEK293T cells were harvested post-transfection and the cell lysates monitored for Cas9 expression by Simple Wes analyses. FIG. 9A demonstrates that plasmids encoding gRNA (G4) targeting the SIN sites in the Cas9 expression vector can inhibit Cas9 expression. The reduction in Cas9 protein levels was quantified within 24, 48, and 72 hours as shown in FIG. 9B. The data demonstrates that the reduction in Cas9 protein levels is a function of gRNA activity since no reduction in Cas9 protein was observed when the Cas9 constructs were transfected alone or in a construct (C0) that did not contain a SIN site. The data shows that the temporal control of Cas9 expression is achieved by co-delivery of self-limiting Cas9 expressing constructs (e.g., expression plasmids containing SIN sites) and plasmids encoding gRNAs that target the SIN site.

Cas9 expressing plasmids containing SIN sites (FIG. 4B: C8) or Cas9 expressing plasmid constructs without SIN sites (FIG. 4A: C0) were transfected alone or co-transfected with plasmids encoding gRNAs (G4 or G5) into HEK293T cells. In this example, G4 expresses a gRNA that does not targets the SIN site in C8; G4 is referred to as a non-targeting gRNA.

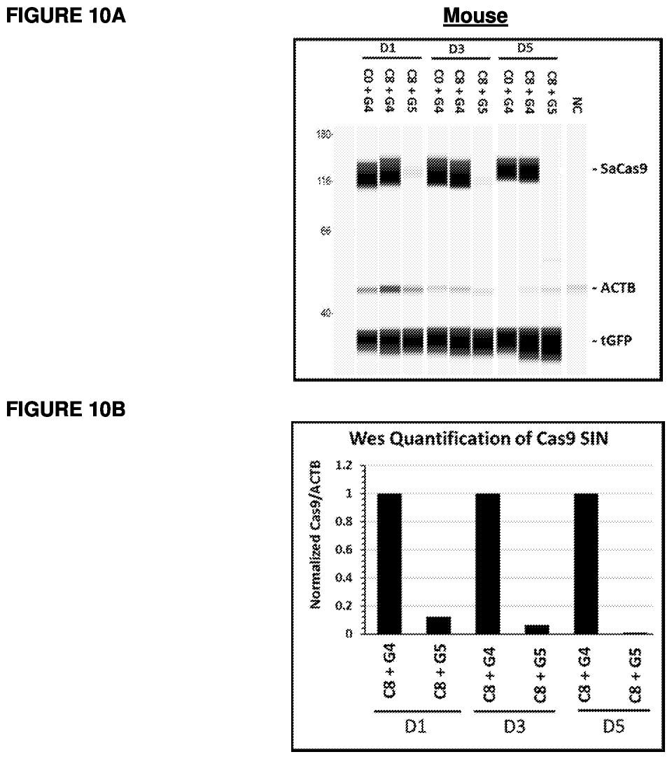

The transfected HEK293T cells were harvested post-transfection and the cell lysates monitored for Cas9 expression by Simple Wes analyses. FIG. 10A demonstrates that plasmids encoding gRNA (G5) targeting the SIN sites in the Cas9 expression vector can inhibit Cas9 expression. However, Cas9 expression is not inhibited from Cas9 constructs without SIN sites (C0) or Cas9 constructs containing SIN sites in the presence of non-targeting gRNAs (G4). Demonstrating that the expression of Cas9 is dependent on the presence of gRNA that targets the SIN site.

The reduction in Cas9 protein levels was again quantified within 24, 72, and 120 hours as shown in FIG. 10B. The data demonstrates that the increased reduction in Cas9 protein levels by time is a function of the activity of a gRNA specifically targeting the SIN site.

### Example 6—Testing On-Target Efficacy Between SaCas9 Constructs

Using the same cell samples from Example 3, cells were harvested at three days post-transfection, genomic DNA was extracted and analyzed for excision of exon 51 (on-target activity) by digital droplet PCR (ddPCR). In brief, genomic DNA extraction was performed using DNeasy kit from Qiagen and were fragmented with HindIII for ˜2 hours. Purified genomic DNA was added into primer/probes mixture and used to generate droplets using autoDG from BioRad (Hercules, Calif.) following manufacture's procedure. DNA droplet samples were then subjected to PCR amplification cycle as follow: 95° C. for 10 mins, 40 cycles of (94° C. for 30secs, 58° C. for 1 mins), 96° C. for 10 mins and 4° C. overnight. DNA quantification and analysis were then completed using ddPCR plate reader and QuantaSoft program. Data presented in FIG. 8C shows that the self-inactivating Cas9 constructs (C2 and C7) have similar editing efficiency (˜25% excision efficiency) as non-self-inactivating Cas9 construct (C0).

In further experiments, using the cell samples from Example 5, cells were harvested at two or three days post-transfection, genomic DNA was extracted and analyzed for excision of exon 51 (on-target activity) by digital droplet PCR (ddPCR) as described above. The data presented in FIG. 9C demonstrates 25%-30% gene editing is achieved in two to three days.

Thus, the cleavage and inactivation of the Cas9 construct does not reduce the ability of modulated Cas9/sgRNA to edit the desired genomic target.

### Example 7—Examining AAV2 Vector SIN Kinetic and On-Target Efficacy

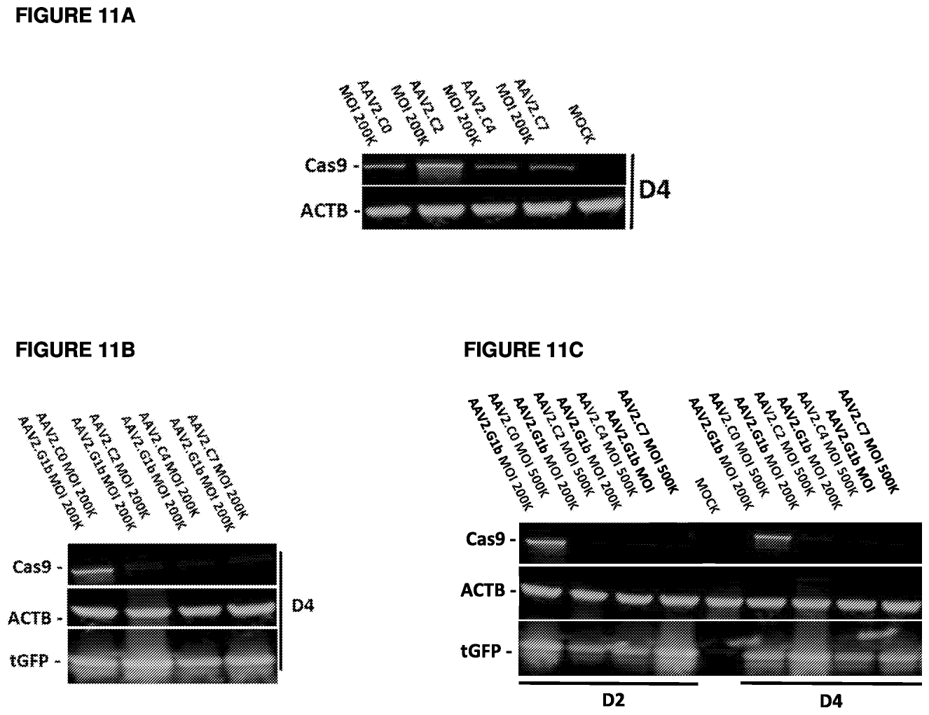

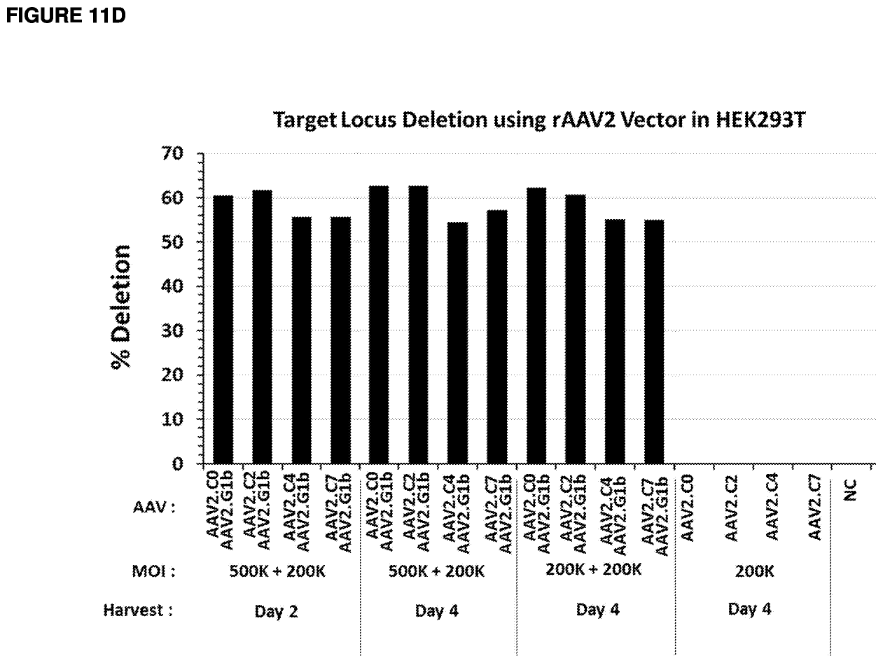

The previous examples demonstrated activity in plasmids. To demonstrate that this self-inactivation system is functional in an AAV system, various Cas9 and gRNA constructs were packaged and produced in recombinant AAV2 vectors. HEK293T cells were then transduced by purified rAAV vectors at different MOI levels (vector genomes/cell) and harvested at different time points (D2: day 2 or D4: day 4). Cas9 was measured using conventional western blot assay. FIG. 11A shows that the AAV vectors express Cas9 protein. However, a substantial reduction of Cas9 protein was observed in the presence of gRNAs that target SIN sites in the construct. For example, AAV2.C2/G1b, AAV2.C4/G1b and AAV2.C7/G1b samples exhibit a reduction in Cas9 protein levels within 2 days (D2) post-infection, as compared to control (AAV2.C0/G1b) as shown in FIG. 11C. Although Cas9 protein expression was significantly inactivated, the efficiency of target locus deletion was not affected. As illustrated by ddPCR, gene editing still occurred at 50%-60% as shown in FIG. 11D.

Similar SIN kinetics and deletion efficacy were observed using various level of equi-MOI or different MOI of dual vectors (FIGS. 11B-11D).

### Example 8—Design, Screen, and Selection of Universal Self-Inactivating Guide RNAs and Target Sites

Candidate universal self-inactivating (SIN) guide RNAs (gRNAs) were screened and selected in a single process or multi-step process that involved theoretical binding. These candidate universal SIN gRNAs were selected based on sequences that match a target site, such as a site within SaCas9, with an adjacent PAM and low potential for cleaving off target sites in the human genome. One or more of a variety of bioinformatics tools available for assessing off-target binding, as described and illustrated in more detail below, was used in order to assess the likelihood of effects at chromosomal positions other than those intended

Candidates predicted to have relatively lower potential for off-target activity can then be assessed experimentally to measure their on-target activity, and then off-target activities at various sites. Preferred guides have sufficiently high on-target activity to achieve desired levels of gene editing at the selected locus, and relatively lower off-target activity to reduce the likelihood of alterations at other chromosomal loci. The ratio of on-target to off-target activity is often referred to as the “specificity” of a guide.

For initial screening of predicted off-target activities, there are a number of bioinformatics tools known and publicly available that can be used to predict the most likely off-target sites; and since binding to target sites in the CRISPR/Cas9 or CRISPR/Cpf1 nuclease system is driven by Watson-Crick base pairing between complementary sequences, the degree of dissimilarity (and therefore reduced potential for off-target binding) is essentially related to primary sequence differences: mismatches and bulges, i.e. bases that are changed to a noncomplementary base, and insertions or deletions of bases in the potential off-target site relative to the target site. An exemplary bioinformatics tool called COSMID (CRISPR Off-target Sites with Mismatches, Insertions and Deletions) (available on the web at crispr.bme.gatech.edu) compiles such similarities. Other bioinformatics tools include, but are not limited to autoCOSMID and CCTop.

Bioinformatics were used to minimize off-target cleavage in order to reduce the detrimental effects of mutations and chromosomal rearrangements. Studies on CRISPR/Cas9 systems suggested the possibility of off-target activity due to non-specific hybridization of the guide strand to DNA sequences with base pair mismatches and/or bulges, particularly at positions distal from the PAM region. Therefore, it is important to have a bioinformatics tool that can identify potential off-target sites that have insertions and/or deletions between the RNA guide strand and genomic sequences, in addition to base-pair mismatches. Bioinformatics tools based upon the off-target prediction algorithm CCTop were used to search genomes for potential CRISPR off-target sites (CCTop is available on the web at crispr.cos.uni-heidelberg.de/). The output ranked lists of the potential off-target sites based on the number and location of mismatches, allowing more informed choice of target sites, and avoiding the use of sites with more likely off-target cleavage.

Additional bioinformatics pipelines were employed that weigh the estimated on and/or off-target activity of gRNA targeting sites in a region. Other features that may be used to predict activity include information about the cell type in question, DNA accessibility, chromatin state, transcription factor binding sites, transcription factor binding data, and other CHIP-seq data. Additional factors were weighed that predict editing efficiency, such as relative positions and directions of pairs of gRNAs, local sequence features and micro-homologies.

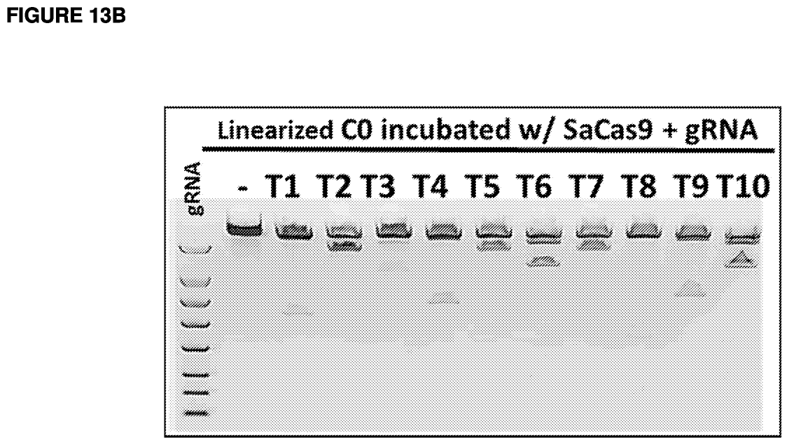

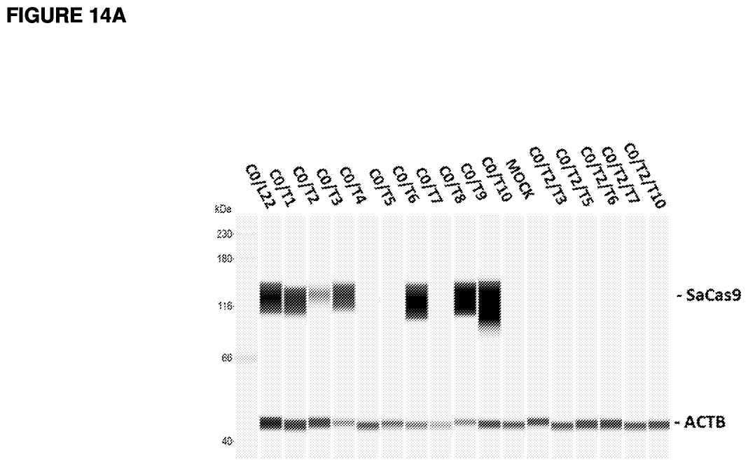

Guide RNAs (gRNAs) that target the SaCas9 sequence can be used to inactivate or modulate expression of SaCas9 (e.g.: universal self-inactivating (SIN) guide RNAs). Codon optimized SaCas9 were screened for particular on-target sites with an adjacent SaCas9 PAM. Initial bioinformatics analysis identified 82 possible candidate gRNAs that matched a sequence within the SaCas9 nucleotide sequence (SEQ ID NO: 79) with adjacent PAM. These sequences were ranked based on the number of off-target sites in the human genome. Guides without, or fewest, target sites in the human genome and those gRNAs having the greatest number of mis-matches were preferentially selected. The top 10 target sites based on this ranking were selected for universal SIN gRNA design. The 10 different SIN sites (T1-T10) are depicted in FIG. 13A and listed in Table 11.

TABLE 11
SIN Sites in Construct C0

| SEQ ID NO: | SIN Site | Sequence |
|---|---|---|
| 63 | T1 | CGTACCGCTTCGACGTGTACCTGGAT |
| 64 | T2 | GGACATCGGAATTACCTCCGTGGGAT |
| 65 | T3 | CGAAACCGGCAGACGAACGAACGGAT |
| 66 | T4 | TGGAGCAGTACGGCGACGAAAAGAAT |
| 67 | T5 | GCCTTTCACGCGGGCTTCGTAGGGGT |
| 68 | T6 | GACAGGATGAAATCGTCCACAAGGGT |
| 69 | T7 | GGGGTTGATACCGGACAATTCCGAGT |
| 70 | T8 | TTGACCTCGTACAGGTTGCCCAGGAT |
| 71 | T9 | TCCCTTGTCGTCCTTGCGCGTGGAGT |
| 72 | T10 | GCGTTGATGACCTTGATCGACTGGAT |

### Example 9—Testing the Functionality of SIN Sites T1-T10

To examine the functionality of universal SIN sites (T1-T10) in the cleaving of SaCas9 constructs, linearized plasmids comprising the SaCas9 nucleotide sequence (SEQ ID NO: 79) were incubated with ribonucleoprotein complexes (RNP) containing purified SaCas9 protein (SEQ ID NO: 1) and synthetic universal SIN gRNAs (where the spacer sequence of the universal SIN gRNAs is complementary to one of SIN sites T1-T10).

Purified plasmid C0 was linearized with PsiI enzyme (New England Biolabs) and purified using ZymoClean DNA gel extraction kit (Zymo Research, Irvine, Calif.). Purified SaCas9 protein was produced by CRO (Aldevron, Madison, Wis.). sgRNAs were chemically synthesized (Integrated DNA Technologies, Coralville, Iowa). For DNA digestion assay, SaCas9, synthetic universal SIN gRNA, and plasmid substrates were mixed in ratio of 10:10:1 and incubated for 2 hours at 37° C. DNA digestion patterns were analyzed using Flash-gel electrophoresis. The resulting products were analyzed by agarose gel electrophoresis.

As shown in FIG. 13B, samples incubated with one of the universal SIN gRNAs that target SIN sites T1-T10, showed the presence of additional bands indicating that all of the synthetic gRNAs tested in associated with Cas9 protein were able to cleave the linearized plasmid. The intensity of the additional bands varied indicating that the efficiency of cutting varied depending on the guide RNA that was used.

### Example 10—Self-Inactivation of SaCas9 Plasmids by Universal SIN gRNAs

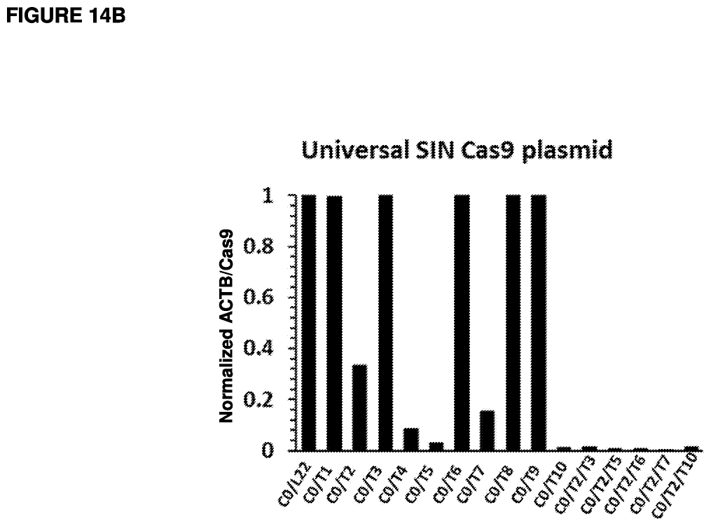

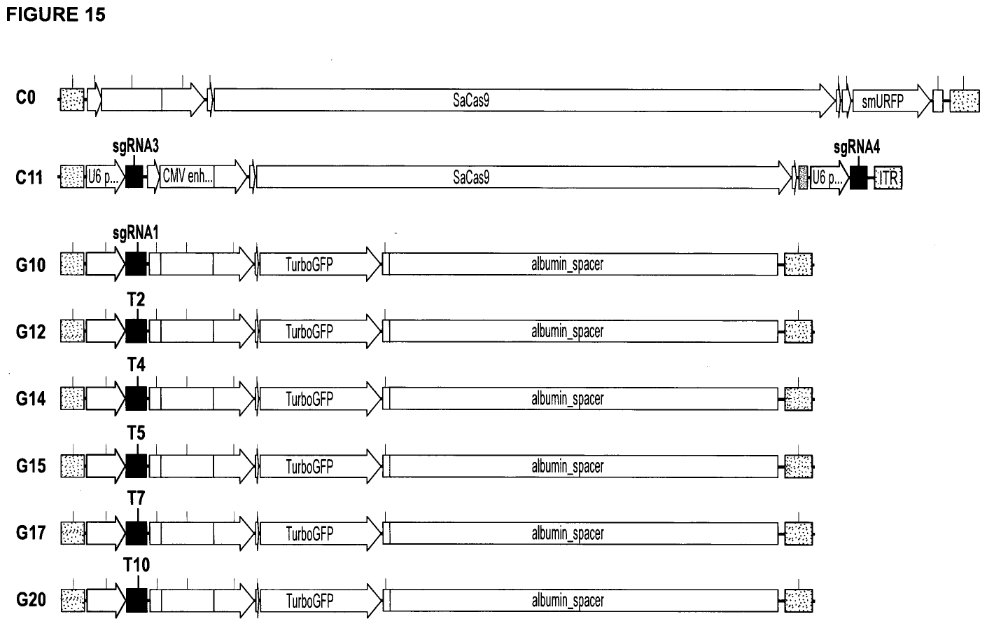

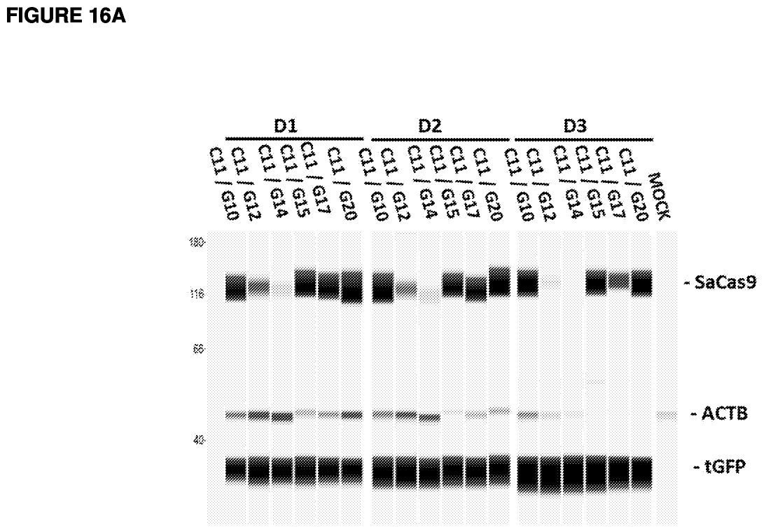

The universal SIN gRNAs were tested to determine their efficiency in inactivating Cas9 activity from expressed plasmids. HEK293 cells were transfected with a plasmid encoding SaCas9-2A-smuRFP (plasmid C0 as shown in FIG. 15) and a plasmid encoding a universal SIN gRNA that targets one of SIN sites T1-T10, using the transfection method described in Example 1. Two days post transfection, the cells were harvested and the lysates measured for Cas9 expression by immunoblot (FIG. 14A) and quantified by densitometry (FIG. 14B). Two days post transfection, the cells were also monitored for RFP expression and gRNAs were ranked according to their self-inactivation potential by scoring the cellular level of RFP expression (data not shown).

These results demonstrate that universal SIN gRNAs T2, T4, T5, T7 and T10 reduce the amount of Cas9 protein expressed in the cell, as shown in FIGS. 14A and 14B. The amount of Cas9 protein is reduced to zero when provided in the following universal SIN gRNA combinations (T2/T3, T2/T5, T2/T6, T2/T7, and T2/T10).

### Example 11—Self-Inactivation of SaCas9 Plasmids Using Universal SIN gRNA Expressing Plasmids

AAV vector plasmid constructs used in these Examples were built using standard cloning procedures and Gibson High-Fidelity assembly reactions based on manufacture's recommendations (New England Biolabs, Ipswich, Mass.).

FIG. 15 depicts plasmid C11, a SaCas9 plasmid construct also containing a guide RNA expression cassette.

FIG. 15 also depicts the schematics of several AAV plasmid constructs that encode universal SIN gRNAs (G12, G14, G15, G17, and G20) (Table 12). The G10 construct expresses a gRNA that targets a site in the human dystrophin locus (sgRNA1, SEQ ID NO: 80) and was used as a control. This construct does not express a universal self-inactivating guide.

TABLE 12
Universal SIN sgRNAs expressed from expression constructs

| Construct | sgRNA | Spacer SEQ ID NO. |
|---|---|---|
| G10 | GUGUAUUGCUUGUACUACUCAguuuaaguacucug | GUGUAUUGCUUG |
| | ugcuggaaacagcacagaaucuacuuaaacaaggcaaaaugccgugu | UACUACUCA |
| | uuaucucgucaacuuguuggcgaga | (SEQ ID NO: 86) |
| | (SEQ ID NO: 73) | |
| G12 | GGACAUCGGAAUUACCUCCGguuuaaguacucugug | GGACAUCGGAAU |
| | cuggaaacagcacagaaucuacuuaaacaaggcaaaaugccguguuu | UACCUCCG |
| | aucucgucaacuuguuggcgaga | (SEQ ID NO: 87) |
| | (SEQ ID NO: 74) | |
| G14 | UGGAGCAGUACGGCGACGAAguuuaaguacucugug | UGGAGCAGUACG |
| | cuggaaacagcacagaaucuacuuaaacaaggcaaaaugccguguuu | GCGACGAA |
| | aucucgucaacuuguuggcgaga | (SEQ ID NO: 88) |
| | (SEQ ID NO: 75) | |
| G15 | GCCUUUCACGCGGGCUUCGUguuuaaguacucugug | GCCUUUCACGCG |
| | cuggaaacagcacagaaucuacuuaaacaaggcaaaaugccguguuu | GGCUUCGU |
| | aucucgucaacuuguuggcgaga | (SEQ ID NO: 89) |
| | (SEQ ID NO: 76) | |
| G17 | GGGGUUGAUACCGGACAAUUguuuaaguacucugu | GGGGUUGAUACC |
| | gcuggaaacagcacagaaucuacuuaaacaaggcaaaaugccguguu | GGACAAUU |
| | uaucucgucaacuuguuggcgaga | (SEQ ID NO: 90) |
| | (SEQ ID NO: 77) | |
| G20 | GCGUUGAUGACCUUGAUCGAguuuaaguacucugug | GCGUUGAUGACC |
| | cuggaaacagcacagaaucuacuuaaacaaggcaaaaugccguguuu | UUGAUCGA |
| | aucucgucaacuuguuggcgaga | (SEQ ID NO: 91) |
| | (SEQ ID NO: 78) | |

*The underlined portion of the sgRNA sequence in Table 12 is the spacer sequence.

These universal SIN gRNAs were used to target the T2, T4, T5, T7, or T10 SIN sites located within the SaCas9 sequence of the C11 plasmid. For example, the G12 construct expresses a universal SIN gRNA that targets the T2 SIN site located within the SaCas9 sequence of C11. The G14 construct expresses a universal SIN gRNA that targets the T4 SIN site located within the SaCas9 sequence of C11. The G15 construct expresses a universal SIN gRNA that targets the T5 SIN site located within the SaCas9 sequence of C11. The G17 construct expresses a universal SIN gRNA that targets the T7 SIN site located within the SaCas9 sequence of C11. The G20 construct expresses a universal SIN gRNA that targets the T10 SIN site located within the SaCas9 sequence of C11. Each universal SIN gRNA construct was generated using a construct that expresses the gRNA backbone (“b”) comprising SEQ ID NOs: 6 or 60.

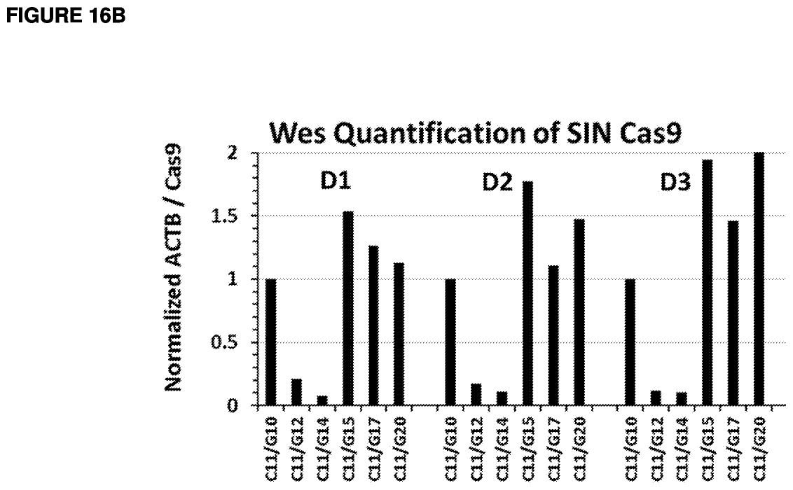

Cas9 expressing plasmids containing SIN sites that correspond to target sites in the human dystrophin locus (FIG. 15: C11) were co-transfected with plasmids encoding universal SIN gRNAs (G12, G14, G15, G17, or G20) or a plasmid that encodes an sgRNA that targets the human dystrophin locus (G10) into HEK293T cells. The transfected HEK293T cells were harvested 24, 48, and 72 hours post-transfection and the cell lysates monitored for Cas9 expression by immunoblot and Simple Wes analyses. FIG. 16A demonstrates that plasmids encoding universal SIN gRNA (G12, G14) targeting the SIN sites in the Cas9 expression vector can reduce Cas9 expression. The reduction in Cas9 protein levels was observed within 24 hours as shown in FIGS. 16A-6B. The data demonstrates that the reduction in Cas9 protein levels is a function of universal SIN gRNA activity since no reduction in Cas9 protein was observed when the Cas9 constructs were transfected with a plasmid that encodes a gRNA that targets the human dystrophin locus. The data shows that the temporal control of Cas9 expression is achieved by co-delivery of Cas9 expressing constructs and plasmids encoding universal SIN gRNAs.

### Example 12—Examining AAV2 Vector SIN Kinetic Efficacy Using Universal SIN gRNAs

The previous examples demonstrated activity in plasmids. To demonstrate that this self-inactivation system is functional in an AAV system, various Cas9 and universal SIN gRNA constructs will be packaged and produced in recombinant AAV2 vectors. HEK293T cells will then be transduced by purified rAAV vectors at different MOI levels (vector genomes/cell) and harvested at different time points (D2: day 2 or D4: day 4). Cas9 will be measured using conventional western blot assay. Results will show that the AAV vectors express Cas9 protein. However, a substantial reduction of Cas9 protein will be observed in the presence of universal SIN gRNAs. Although Cas9 protein expression will be significantly inactivated, the efficiency of target locus deletion will not be affected.

TABLE 13
Listing of guide RNA nucleotide sequences useful for generating the
plasmid and AAV constructs expression constructs

| Guide RNA name | Guide RNA DNA sequence | SEQ ID NO: |
|---|---|---|
| sgRNA1 | GTGTATTGCTTGTACTACTCAGTTTTAGTA | 121 |
| | CTCTGTAATGAAAATTACAGAATCTACTAA | |
| | AACAAGGCAAAATGCCGTGTTTATCTCGTC | |
| | AACTTGTTGGCGAGA | |
| sgRNA2 | GTGTTATTACTTGCTACTGCAGTTTTAGTA | 122 |
| | CTCTGTAATGAAAATTACAGAATCTACTAA | |
| | AACAAGGCAAAATGCCGTGTTTATCTCGTC | |
| | AACTTGTTGGCGAGA | |
| sgRNA1 | GTGTATTGCTTGTACTACTCAGTTTAAGTA | 123 |
| | CTCTGTGCTGGAAACAGCACAGAATCTACT | |
| | TAAACAAGGCAAAATGCCGTGTTTATCTCG | |
| | TCAACTTGTTGGCGAGA | |
| sgRNA2 | GTGTTATTACTTGCTACTGCAGTTTAAGTA | 124 |
| | CTCTGTGCTGGAAACAGCACAGAATCTACT | |
| | TAAACAAGGCAAAATGCCGTGTTTATCTCG | |
| | TCAACTTGTTGGCGAGA | |
| sgRNA3 | GCTTAGAGGTCTTCTACATACAGTTTAAGT | 125 |
| | ACTCTGTGCTGGAAACAGCACAGAATCTA | |
| | CTTAAACAAGGCAAAATGCCGTGTTTATCT | |
| | CGTCAACTTGTTGGCGAGA | |
| sgRNA4 | GCTATTCTGAGTACAGAGCATAGTTTAAGT | 126 |
| | ACTCTGTGCTGGAAACAGCACAGAATCTA | |
| | CTTAAACAAGGCAAAATGCCGTGTTTATCT | |
| | CGTCAACTTGTTGGCGAGA | |
| sgRNA5 | GACTATGATTAAATGCTTGATAGTTTAAGT | 127 |
| | ACTCTGTGCTGGAAACAGCACAGAATCTA | |
| | CTTAAACAAGGCAAAATGCCGTGTTTATCT | |
| | CGTCAACTTGTTGGCGAGA | |
| sgRNA6 | GCTTAAAGGCTTCATATAAGGGGTTTAAGT | 128 |
| | ACTCTGTGCTGGAAACAGCACAGAATCTA | |
| | CTTAAACAAGGCAAAATGCCGTGTTTATCT | |
| | CGTCAACTTGTTGGCGAGA | |
| gT2 | GGACATCGGAATTACCTCCGGTTTAAGTAC | 129 |
| | TCTGTGCTGGAAACAGCACAGAATCTACTT | |
| | AAACAAGGCAAAATGCCGTGTTTATCTCGT | |
| | CAACTTGTTGGCGAGA | |
| gT4 | GTGGAGCAGTACGGCGACGAAGTTTAAGT | 130 |
| | ACTCTGTGCTGGAAACAGCACAGAATCTA | |
| | CTTAAACAAGGCAAAATGCCGTGTTTATCT | |
| | CGTCAACTTGTTGGCGAGA | |
| gT5 | GCCTTTCACGCGGGCTTCGTGTTTAAGTAC | 131 |
| | TCTGTGCTGGAAACAGCACAGAATCTACTT | |
| | AAACAAGGCAAAATGCCGTGTTTATCTCGT | |
| | CAACTTGTTGGCGAGA | |
| gT7 | GGGGTTGATACCGGACAATTGTTTAAGTAC | 132 |
| | TCTGTGCTGGAAACAGCACAGAATCTACTT | |
| | AAACAAGGCAAAATGCCGTGTTTATCTCGT | |
| | CAACTTGTTGGCGAGA | |
| gT10 | GCGTTGATGACCTTGATCGAGTTTAAGTAC | 133 |
| | TCTGTGCTGGAAACAGCACAGAATCTACTT | |
| | AAACAAGGCAAAATGCCGTGTTTATCTCGT | |
| | CAACTTGTTGGCGAGA | |
| Guide RNA T1 | CGTACCGCTTCGACGTGTACGTTTAAGTAC | 134 |
| | TCTGTGCTGGAAACAGCACAGAATCTACTT | |
| | AAACAAGGCAAAATGCCGTGTTTATCTCGT | |
| | CAACTTGTTGGCGAGA | |
| Guide RNA T3 | CGAAACCGGCAGACGAACGAGTTTAAGTA | 135 |
| | CTCTGTGCTGGAAACAGCACAGAATCTACT | |
| | TAAACAAGGCAAAATGCCGTGTTTATCTCG | |
| | TCAACTTGTTGGCGAGA | |
| Guide RNA T6 | GACAGGATGAAATCGTCCACGTTTAAGTA | 136 |
| | CTCTGTGCTGGAAACAGCACAGAATCTACT | |
| | TAAACAAGGCAAAATGCCGTGTTTATCTCG | |
| | TCAACTTGTTGGCGAGA | |
| Guide RNA T8 | TTGACCTCGTACAGGTTGCCGTTTAAGTAC | 137 |
| | TCTGTGCTGGAAACAGCACAGAATCTACTT | |
| | AAACAAGGCAAAATGCCGTGTTTATCTCGT | |
| | CAACTTGTTGGCGAGA | |
| Guide RNA T9 | TCCCTTGTCGTCCTTGCGCGGTTTAAGTAC | 138 |
| | TCTGTGCTGGAAACAGCACAGAATCTACTT | |
| | AAACAAGGCAAAATGCCGTGTTTATCTCGT | |
| | CAACTTGTTGGCGAGA | |

*Spacer sequence is underlined.

### Note Regarding Illustrative Examples and Documents Cited

While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed, and not as more narrowly defined by particular illustrative aspects provided herein.

Any patent, publication, or other disclosure material identified herein is incorporated by reference into this specification in its entirety unless otherwise indicated, but only to the extent that the incorporated material does not conflict with existing descriptions, definitions, statements, or other disclosure material expressly set forth in this specification. As such, and to the extent necessary, the express disclosure as set forth in this specification supersedes any conflicting material incorporated by reference. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.
1. Cho, S. W., Kim, S., Kim, J. M. and Kim, J. S. (2013) Targeted genome engineering in human cells with the Cas9 RNA-guided endonuclease. Nat. Biotechnol., 31, 230-232.
2. Cong, L., Ran, F. A., Cox, D., Lin, S., Barretto, R., Habib, N., Hsu, P. D., Wu, X., Jiang, W., Marraffini, L. A. et al. (2013) Multiplex genome engineering using CRISPR/Cas systems. Science, 339, 819-823.
3. DiCarlo, J. E., Norville, J. E., Mali, P., Rios, X., Aach, J. and Church, G. M. (2013) Genome engineering in Saccharomyces cerevisiae using CRISPR-Cas systems. Nucleic Acids Res., 41, 4336-4343.
4. Friedland, A. E., Tzur, Y. B., Esvelt, K. M., Colaiacovo, M. P., Church, G. M. and Calarco, J. A. (2013) Heritable genome editing in C. elegans via a CRISPR-Cas9 system. Nat. Methods, 10, 741-743.
5. Gratz, S. J., Cummings, A. M., Nguyen, J. N., Hamm, D. C., Donohue, L. K., Harrison, M. M., Wildonger, J. and O'Connor-Giles, K. M. (2013) Genome engineering of Drosophila with the CRISPR RNA-guided Cas9 nuclease. Genetics, 194, 1029-1035.
6. Hwang, W. Y., Fu, Y., Reyon, D., Maeder, M. L., Tsai, S. Q., Sander, J. D., Peterson, R. T., Yeh, J. R. and Joung, J. K. (2013) Efficient genome editing in zebrafish using a CRISPR-Cas system. Nat. Biotechnol., 31, 227-229.
7. Jiang, W., Bikard, D., Cox, D., Zhang, F. and Marraffini, L. A. (2013) RNA-guided editing of bacterial genomes using CRISPR-Cas systems. Nat. Biotechnol., 31, 233-239.
8. Mali, P., Yang, L., Esvelt, K. M., Aach, J., Guell, M., Dicarlo, J. E., Norville, J. E. and Church, G. M. (2013) RNA-guided human genome engineering via Cas9. Science, 339, 823-826.
9. Shen, B., Zhang, J., Wu, H., Wang, J., Ma, K., Li, Z., Zhang, X., Zhang, P. and Huang, X. (2013) Generation of gene-modified mice via Cas9/RNA-mediated gene targeting. Cell Res., 23, 720-723.
10. Wang, H., Yang, H., Shivalila, C. S., Dawlaty, M. M., Cheng, A. W., Zhang, F. and Jaenisch, R. (2013) One-step generation of mice carrying mutations in multiple genes by CRISPR/Cas-mediated genome engineering. Cell, 153, 910-918.
11. Jinek, M., East, A., Cheng, A., Lin, S., Ma, E. and Doudna, J. (2013) RNA-programmed genome editing in human cells. eLIFE, 2, e00471.
12. Li, J. F., Norville, J. E., Aach, J., McCormack, M., Zhang, D., Bush, J., Church, G. M. and Sheen, J. (2013) Multiplex and homologous recombination-mediated genome editing in Arabidopsis and Nicotiana benthamiana using guide RNA and Cas9. Nat. Biotechnol., 31, 688-691.
13. Nekrasov, V., Staskawicz, B., Weigel, D., Jones, J. D. and Kamoun, S. (2013) Targeted mutagenesis in the model plant Nicotiana benthamiana using Cas9 RNA-guided endonuclease. Nat. Biotechnol., 31, 691-693.
14. Jinek, M., Chylinski, K., Fonfara, I., Hauer, M., Doudna, J. A. and Charpentier, E. (2012) A programmable dual-RNA-guided DNA endonuclease in adaptive bacterial immunity. Science, 337, 816-821.
15. Chylinski, K., Le Rhun, A. and Charpentier, E. (2013) The tracrRNA and Cas9 families of type II CRISPR-Cas immunity systems. RNA Biol., 10, 726-737.
16. Deltcheva, E., Chylinski, K., Sharma, C. M., Gonzales, K., Chao, Y., Pirzada, Z. A., Eckert, M. R., Vogel, J. and Charpentier, E. (2011) CRISPR RNA maturation by trans-encoded small RNA and host factor RNase III. Nature, 471, 602-607.
17. Karvelis, T., Gasiunas, G., Miksys, A., Barrangou, R., Horvath, P. and Siksnys, V. (2013) crRNA and tracrRNA guide Cas9-mediated DNA interference in Streptococcus thermophilus. RNA Biol., 10, 841-851.
18. Garneau, J. E., Dupuis, M. E., Villion, M., Romero, D. A., Barrangou, R., Boyaval, P., Fremaux, C., Horvath, P., Magadan, A. H. and Moineau, S. (2010) The CRISPR/Cas bacterial immune system cleaves bacteriophage and plasmid DNA. Nature, 468, 67-71.
19. Magadan, A. H., Dupuis, M. E., Villion, M. and Moineau, S. (2012) Cleavage of phage DNA by the Streptococcus thermophilus CRISPR3-Cas system. PLoS One, 7, e40913.
20. Haft, D. H., Selengut, J., Mongodin, E. F. and Nelson, K. E. (2005) A guild of 45 CRISPR-associated (Cas) protein families and multiple CRISPR/Cas subtypes exist in prokaryotic genomes. PLoS Comput. Biol., 1, e60.
21. Makarova, K. S., Grishin, N. V., Shabalina, S. A., Wolf, Y. I. and Koonin, E. V. (2006) A putative RNA-interference-based immune system in prokaryotes: computational analysis of the predicted enzymatic machinery, functional analogies with eukaryotic RNAi, and hypothetical mechanisms of action. Biol. Direct, 1, 7.
22. Gasiunas, G., Barrangou, R., Horvath, P. and Siksnys, V. (2012) Cas9-crRNA ribonucleoprotein complex mediates specific DNA cleavage for adaptive immunity in bacteria. Proc. Natl. Acad. Sci. U.S.A, 109, E2579-2586.
23. Sapranauskas, R., Gasiunas, G., Fremaux, C., Barrangou, R., Horvath, P. and Siksnys, V. (2011) The Streptococcus thermophilus CRISPR/Cas system provides immunity in Escherichia coli. Nucleic Acids Res., 39, 9275-9282.
24. Mali, P., Aach, J., Stranges, P. B., Esvelt, K. M., Moosburner, M., Kosuri, S., Yang, L. and Church, G. M. (2013) Cas9 transcriptional activators for target specificity screening and paired nickases for cooperative genome engineering. Nat Biotechnol., 31, 833-838.
25. Ran, F. A., Hsu, P. D., Lin, C. Y., Gootenberg, J. S., Konermann, S., Trevino, A. E., Scott, D. A., Inoue, A., Matoba, S., Zhang, Y. et al. (2013) Double nicking by RNA-guided CRISPR Cas9 for enhanced genome editing specificity. Cell, 154, 1380-1389.
26. Deveau, H., Barrangou, R., Garneau, J. E., Labonte, J., Fremaux, C., Boyaval, P., Romero, D. A., Horvath, P. and Moineau, S. (2008) Phage response to CRISPR-encoded resistance in Streptococcus thermophilus. J. Bacteriol., 190, 1390-1400.
27. Horvath, P., Romero, D. A., Coute-Monvoisin, A. C., Richards, M., Deveau, H., Moineau, S., Boyaval, P., Fremaux, C. and Barrangou, R. (2008) Diversity, activity, and evolution of CRISPR loci in Streptococcus thermophilus. J. Bacteriol., 190, 1401-1412.
28. Mojica, F. J., Diez-Villasenor, C., Garcia-Martinez, J. and Almendros, C. (2009) Short motif sequences determine the targets of the prokaryotic CRISPR defence system. Microbiology, 155, 733-740.
29. Bikard, D., Jiang, W., Samai, P., Hochschild, A., Zhang, F. and Marraffini, L. A. (2013) Programmable repression and activation of bacterial gene expression using an engineered CRISPR-Cas system. Nucleic Acids Res., 41, 7429-7437.
30. Qi, L. S., Larson, M. H., Gilbert, L. A., Doudna, J. A., Weissman, J. S., Arkin, A. P. and Lim, W. A. (2013) Repurposing CRISPR as an RNA-guided platform for sequence-specific control of gene expression. Cell, 152, 1173-1183.
31. Charpentier, E. and Doudna, J. A. (2013) Biotechnology: Rewriting a genome. Nature, 495, 50-51.
32. Horvath, P. and Barrangou, R. (2013) RNA-guided genome editing a la carte. Cell Res., 23, 733-734.
33. van der Oost, J. (2013) Molecular biology. New tool for genome surgery. Science, 339, 768-770.
34. Hou, Z., Zhang, Y., Propson, N. E., Howden, S. E., Chu, L. F., Sontheimer, E. J. and Thomson, J. A. (2013) Efficient genome engineering in human pluripotent stem cells using Cas9 from Neisseria meningitidis. Proc. Natl. Acad. Sci. U.S.A, 110, 15644-15649.
35. Sambrook, J., Fritsch, E. F. and Maniatis, T. (1989) Molecular Cloning: a Laboratory Manual. 2nd edn. Cold Spring Harbor, N. Y. ed. Cold Spring Harbor Laboratory Press.
36. Caparon, M. G. and Scott, J. R. (1991) Genetic manipulation of pathogenic streptococci. Methods Enzymol., 204, 556-586.
37. Kirsch, R. D. and Joly, E. (1998) An improved PCR-mutagenesis strategy for two-site mutagenesis or sequence swapping between related genes. Nucleic Acids Res., 26, 1848-1850.
38. Siller, M., Janapatla, R. P., Pirzada, Z. A., Hassler, C., Zinkl, D. and Charpentier, E. (2008) Functional analysis of the group A streptococcal luxS/AI-2 system in metabolism, adaptation to stress and interaction with host cells. BMC Microbiol., 8, 188.
39. Mangold, M., Siller, M., Roppenser, B., Vlaminckx, B. J., Penfound, T. A., Klein, R., Novak, R., Novick, R. P. and Charpentier, E. (2004) Synthesis of group A streptococcal virulence factors is controlled by a regulatory RNA molecule. Mol. Microbiol., 53, 1515-1527.
40. Herbert, S., Barry, P. and Novick, R. P. (2001) Subinhibitory clindamycin differentially inhibits transcription of exoprotein genes in Staphylococcus aureus. Infect. Immun., 69, 2996-3003.
41. Pall, G. S. and Hamilton, A. J. (2008) Improved northern blot method for enhanced detection of small RNA. Nat. Protoc., 3, 1077-1084.
42. Urban, J. H. and Vogel, J. (2007) Translational control and target recognition by Escherichia coli small RNAs in vivo. Nucleic Acids Res., 35, 1018-1037.
43. McClelland, M., Hanish, J., Nelson, M. and Patel, Y. (1988) KGB: a single buffer for all restriction endonucleases. Nucleic Acids Res., 16, 364.
44. Makarova, K. S., Haft, D. H., Barrangou, R., Brouns, S. J., Charpentier, E., Horvath, P., Moineau, S., Mojica, F. J., Wolf, Y. I., Yakunin, A. F. et al. (2011) Evolution and classification of the CRISPR-Cas systems. Nat. Rev. Microbiol., 9, 467-477.
45. Altschul, S. F., Madden, T. L., Schaffer, A. A., Zhang, J., Zhang, Z., Miller, W. and Lipman, D. J. (1997) Gapped BLAST and PSI-BLAST: a new generation of protein database search programs. Nucleic Acids Res., 25, 3389-3402.
46. Wheeler, D. and Bhagwat, M. (2007) BLAST QuickStart: example-driven web-based BLAST tutorial. Methods Mol. Biol., 395, 149-176.
47. Edgar, R. C. (2004) MUSCLE: multiple sequence alignment with high accuracy and high throughput. Nucleic Acids Res., 32, 1792-1797.
48. Soding, J., Biegert, A. and Lupas, A. N. (2005) The HHpred interactive server for protein homology detection and structure prediction. Nucleic Acids Res., 33, W244-248.
49. Price, M. N., Dehal, P. S. and Arkin, A. P. (2010) FastTree 2—approximately maximum-likelihood trees for large alignments. PLoS One, 5, e9490.
50. Bernhart, S. H., Tafer, H., Muckstein, U., Flamm, C., Stadler, P. F. and Hofacker, I. L. (2006) Partition function and base pairing probabilities of RNA heterodimers. Algorithms Mol. Biol., 1, 3.
51. Hofacker, I. L., Fekete, M. and Stadler, P. F. (2002) Secondary structure prediction for aligned RNA sequences. Journal of molecular biology, 319, 1059-1066.
52. Darty, K., Denise, A. and Ponty, Y. (2009) VARNA: Interactive drawing and editing of the RNA secondary structure. Bioinformatics, 25, 1974-1975.
53. Bhaya, D., Davison, M. and Barrangou, R. (2011) CRISPR-Cas systems in bacteria and archaea: versatile small RNAs for adaptive defense and regulation. Annu. Rev. Genet., 45, 273-297.
54. Zhang, Y., Heidrich, N., Ampattu, B. J., Gunderson, C. W., Seifert, H. S., Schoen, C.,
Vogel, J. and Sontheimer, E. J. (2013) Processing-independent CRISPR RNAs limit natural transformation in Neisseria meningitidis. Mol. Cell, 50, 488-503.
55. Takeuchi, N., Wolf, Y. I., Makarova, K. S. and Koonin, E. V. (2012) Nature and intensity of selection pressure on CRISPR-associated genes. J. Bacteriol., 194, 1216-1225.
56. Makarova, K. S., Aravind, L., Wolf, Y. I. and Koonin, E. V. (2011) Unification of Cas protein families and a simple scenario for the origin and evolution of CRISPR-Cas systems. Biol. Direct., 6, 38.
57. Barrangou, R., Fremaux, C., Deveau, H., Richards, M., Boyav al, P., Moineau, S., Romero, D. A. and Horvath, P. (2007) CRISPR provides acquired resistance against viruses in prokaryotes. Science, 315, 1709-1712.
58. Sun, W., Li, G. and Nicholson, A. W. (2004) Mutational analysis of the nuclease domain of Escherichia coli ribonuclease III. Identification of conserved acidic residues that are important for catalytic function in vitro. Biochemistry, 43, 13054-13062.
59. Sun, W., Jun, E. and Nicholson, A. W. (2001) Intrinsic double-stranded-RNA processing activity of Escherichia coli ribonuclease III lacking the dsRNA-binding domain. Biochemistry, 40, 14976-14984.

1. A self-inactivating CRISPR-Cas system comprising:
(a) a first nucleic acid segment comprising a codon optimized nucleotide sequence encoding a site-directed Cas9 polypeptide or variant thereof, wherein the codon optimized sequence comprises a first self-inactivating (SIN) site and an adjacent Protospacer Adjacent Motif (PAM) within the open reading frame (ORF), and wherein the first SIN site is the result of codon optimization; and
(b) a second nucleic acid segment comprising a nucleotide sequence encoding a guide RNA (gRNA), wherein the gRNA comprises a DNA-targeting sequence that is complementary to the self-inactivating (SIN) site in the first nucleic acid segment,
wherein the gRNA guides the Cas9 polypeptide or variant thereof to cleave the first nucleic acid segment at the SIN site within the codon optimized sequence and reduces expression of the site directed Cas9 polypeptide or variant thereof.

2. The self-inactivating CRISPR-Cas system of claim 1, wherein the first nucleic acid segment comprises:
(i) a nucleotide sequence encoding a first target gRNA that comprises a DNA-targeting sequence that is complementary to a nucleotide sequence present in a target gene in a cell: or
(ii) a nucleotide sequence encoding a first target gRNA that comprises a DNA-targeting sequence that is complementary to a nucleotide sequence present in a target gene in a cell and a nucleotide sequence that encodes a second target gRNA that comprises a DNA-targeting sequence that is complementary to a nucleotide sequence present in a target gene in a cell.

3. (canceled)

4. The self-inactivating CRISPR-Cas system of claim 2, wherein the first SIN site in the first nucleic acid segment is located:
(a) at the 5′ end of the nucleotide sequence encoding the Cas9 polypeptide or variant thereof;
(b) at the 3′ end of the nucleotide sequence encoding the Cas9 polypeptide or variant thereof; or
(c) in an intron within the nucleotide sequence encoding the Cas9 polypeptide or variant thereof.

5. The self-inactivating CRISPR-Cas system of claim 2, wherein the nucleotide sequence encoding the first and/or second target gRNA in the first nucleic acid segment is located:
(a) at the 5′ end of the nucleotide sequence encoding the Cas9 polypeptide or variant thereof; or
(b) at the 3′ end of the nucleotide sequence encoding the Cas9 polypeptide or variant thereof.

6. The self-inactivating CRISPR-Cas system of claim 5, wherein the nucleotide sequence encoding the first target gRNA is located at the 5′ end of the nucleotide sequence encoding the Cas9 polypeptide or variant thereof, and the second target gRNA is located at the 3′ end of the of the nucleotide sequence encoding the site-directed Cas9 polypeptide or variant thereof.

7. The self-inactivating CRISPR-Cas system of claim 1, wherein the codon optimized sequence comprises a second SIN site, and wherein:
(i) the nucleotide sequence of the first SIN site and the second SIN site are the same; or
(ii) the first target SIN site and the second target SIN site are different.

8. (canceled)

9. The self-inactivating CRISPR-Cas system of claim 1, wherein the gRNA is a two-molecule guide RNA, or a single RNA molecule.

10.-11. (canceled)

12. The self-inactivating CRISPR-Cas system of claim 2, wherein the first target gRNA is a two-molecule guide RNA or a single RNA molecule, and wherein the second target gRNA is a two-molecule guide RNA or a single RNA molecule.

13.-17. (canceled)

18. The self-inactivating CRISPR-Cas system of claim 1, wherein the self-inactivating CRISPR-Cas system comprises:
(i) a first vector comprising the first nucleic acid segment, and a second vector comprising the second nucleic acid segment; or
(ii) a first vector comprising the first and second nucleic acid segments.

19. (canceled)

20. The self-inactivating CRISPR-Cas system of claim 18, wherein at least one of the first vector and the second vector is an adeno-associated virus (AAV) vector, or wherein the vector is AAV2.

21. (canceled)

22. The self-inactivating CRISPR-Cas system of claim 1, wherein the site-directed Cas9 polypeptide is Staphylococcus aureus Cas9 (SaCas9) or a variant thereof; or wherein the site-directed Cas9 polypeptide comprises the amino acid sequence set forth in SEQ ID NO: 1, SEQ ID NO: 2, SEQ ID NO: 3 or SEQ ID NO: 4.

23. (canceled)

24. The self-inactivating CRISPR-Cas system of claim 1, wherein the nucleotide sequence that encodes the site-directed Cas9 polypeptide comprises SEQ ID NO: 79.

25. The self-inactivating CRISPR-Cas system of claim 1, wherein the PAM sequence adjacent the SIN site is selected from the group consisting of: NNGRRT, NRG, NAAAAN, NAAAAC, NNNNGHTT, YTN, NNNNACA, NNNACAC, NNVRYAC, NNNVRYM, NNAAAAW, or NNAGAAW.

26. The self-inactivating CRISPR-Cas system of claim 1, wherein the nucleotide sequence of the SIN site within the nucleotide sequence encoding the Cas9 polypeptide or variant thereof comprises a nucleotide sequence selected from the group consisting of SEQ ID NO: 63-72.

27. The self-inactivating CRISPR-Cas system of claim 1, wherein the nucleotide sequence of the SIN site is less than 25 nucleotides in length.

28.-29. (canceled)

30. The self-inactivating CRISPR-Cas system of claim 1, further comprising a second SIN site within the nucleotide sequence encoding the Cas9 polypeptide or variant thereof as a result of codon optimization.

31. The self-inactivating CRISPR-Cas system of claim 30, wherein the second SIN site comprises a nucleotide sequence selected from the group consisting of SEQ ID NOs: 63-72.

32. The self-inactivating CRISPR-Cas system of claim 30, wherein the first SIN site comprises the nucleotide sequence of SEQ ID NO: 64, and the second SIN site comprises a nucleotide sequence selected from SEQ ID NOs: 65-72.

33. (canceled)

34. The self-inactivating CRISPR-Cas system of claim 1, wherein
(a) the SIN site within the nucleotide sequence encoding the Cas9 polypeptide or variant thereof comprises the nucleotide sequence of SEQ ID NO: 64, and the DNA-targeting segment of the gRNA which is complementary to the SIN site comprises the nucleotide sequence of SEQ ID NO: 87;
(b) the SIN site within the nucleotide sequence encoding the Cas9 polypeptide or variant thereof comprises the nucleotide sequence of SEQ ID NO: 66, and the DNA-targeting segment of the gRNA which is complementary to the SIN site comprises the nucleotide sequence of SEQ ID NO: 88;
(c) the SIN site within the nucleotide sequence encoding the Cas9 polypeptide or variant thereof comprises the nucleotide sequence of SEQ ID NO: 67, and the DNA-targeting segment of the gRNA which is complementary to the SIN site comprises the nucleotide sequence of SEQ ID NO: 89;
(d) the SIN site within the nucleotide sequence encoding the Cas9 polypeptide or variant thereof comprises the nucleotide sequence of SEQ ID NO: 69, and the DNA-targeting segment of the gRNA which is complementary to the SIN site comprises the nucleotide sequence of SEQ ID NO: 90;
(e) the SIN site within the nucleotide sequence encoding the Cas9 polypeptide or variant thereof comprises the nucleotide sequence of SEQ ID NO: 72, and the DNA-targeting segment of the gRNA which is complementary to the SIN site comprises the nucleotide sequence of SEQ ID NO: 91;
(f) the SIN site within the nucleotide sequence encoding the Cas9 polypeptide or variant thereof comprises the nucleotide sequence of SEQ ID NO: 64, and the gRNA which is complementary to the SIN site comprises the nucleotide sequence of SEQ ID NO: 74;
(g) the SIN site within the nucleotide sequence encoding the Cas9 polypeptide or variant thereof comprises the nucleotide sequence of SEQ ID NO: 66, and the gRNA which is complementary to the SIN site comprises the nucleotide sequence of SEQ ID NO: 75;
(h) the SIN site within the nucleotide sequence encoding the Cas9 polypeptide or variant thereof comprises the nucleotide sequence of SEQ ID NO: 67, and the gRNA which is complementary to the SIN site comprises the nucleotide sequence of SEQ ID NO: 76;
(i) the SIN site within the nucleotide sequence encoding the Cas9 polypeptide or variant thereof comprises the nucleotide sequence of SEQ ID NO: 69, and the gRNA which is complementary to the SIN site comprises the nucleotide sequence of SEQ ID NO: 77; or
(j) the SIN site within the nucleotide sequence encoding the Cas9 polypeptide or variant thereof comprises the nucleotide sequence of SEQ ID NO: 72, and the gRNA which is complementary to the SIN site comprises the nucleotide sequence of SEQ ID NO: 78.

35. (canceled)

36. A self-inactivating CRISPR-Cas system comprising:
(a) a first nucleic acid segment comprising (i) a nucleotide sequence that encodes a site-directed Cas9 polypeptide or variant thereof, and (ii) a chimeric intron comprising a self-inactivating (SIN) site; and
(b) a second nucleic acid segment comprising a nucleotide sequence that encodes a guide RNA (gRNA), wherein the gRNA comprises a DNA-targeting sequence that is complementary to a SIN site in the first nucleic acid segment.

37. The self-inactivating CRISPR-Cas system of claim 36, wherein the chimeric intron is inserted into the Cas9 open reading frame (ORF).

38. The self-inactivating CRISPR-Cas system of claim 37, wherein the chimeric intron is inserted before or after the codon encoding amino acid N580 of the Cas9 polypeptide or variant thereof, or wherein the chimeric intron is inserted before or after the codon encoding amino acid D10 of the Cas9 polypeptide or variant thereof.

39. (canceled)

40. The self-inactivating CRISPR-Cas system of claim 36, wherein the chimeric intron comprises a 5′-donor site from the first intron of the human β-globin gene and the branch and 3′-acceptor site from the intron of an immunoglobulin heavy chain variable region.

41. The self-inactivating CRISPR-Cas system of claim 36, wherein the SIN site in the chimeric intron is complementary to the DNA-targeting sequence of a first target gRNA that binds to a target gene in the cell, or wherein the SIN site comprises a nucleotide sequence selected from the group consisting of SEQ ID NOs: 114, 115, 116, 118 or 120.

42. (canceled)

43. The self-inactivating CRISPR-Cas system of claim 41, wherein the first nucleic acid segment comprises a second SIN site.

44. The self-inactivating CRISPR-Cas system of claim 43, wherein the second SIN site is located:
(a) at the 5′ end of the nucleotide sequence encoding the Cas9 polypeptide or variant thereof;
(b) at the 3′ end of the nucleotide sequence encoding the Cas9 polypeptide or variant thereof;
(c) within the nucleotide sequence encoding the Cas9 polypeptide or variant thereof.

45. The self-inactivating CRISPR-Cas system of claim 43, wherein the nucleotide sequence of the first and second SIN sites in the first nucleic acid segment are the same or different.

46. (canceled)

47. The self-inactivating CRISPR-Cas system of claim 45, wherein the second SIN site is complementary to the DNA-targeting sequence of a second target gRNA that binds to the target gene in the cell.

48. The self-inactivating CRISPR-Cas system of claim 47, wherein the second SIN site is located at the 5′ end of the nucleotide sequence encoding the Cas9 polypeptide or variant thereof, wherein the second SIN site is located at the 3′ end of the nucleotide sequence encoding the Cas9 polypeptide or variant thereof, or wherein the second SIN site is located within the nucleotide sequence encoding the Cas9 polypeptide or variant thereof.

49.-50. (canceled)

51. The self-inactivating CRISPR-Cas system of claim 48, wherein the second SIN site is within the open reading frame (ORF) of the nucleotide sequence encoding the Cas9 polypeptide or variant thereof.

52. The self-inactivating CRISPR-Cas system of claim 36, wherein the gRNA that is complementary to the SIN site in the chimeric intron is a two-molecule guide RNA, or a single RNA molecule.

53.-54. (canceled)

55. The self-inactivating CRISPR-Cas system of claim 43, wherein the gRNA that is complementary to the second SIN site in the first nucleic acid segment is a two-molecule guide RNA, or a single RNA molecule.

56.-57. (canceled)

58. The self-inactivating CRISPR-Cas system of claim 36, wherein the self-inactivating CRISPR-Cas system comprises;
(i) a first vector comprising the first nucleic acid segment, and a second vector comprising the second nucleic acid segment; or
(ii) a first vector comprising the first and second nucleic acid segments.

59. (canceled)

60. The self-inactivating CRISPR-Cas system of claim 58, wherein the second vector comprises a second nucleotide sequence that encodes a second target gRNA that comprises a DNA-targeting sequence that is complementary to the target gene in a cell.

61. The self-inactivating CRISPR-Cas system of claim 58, wherein at least one of the first vector and the second vector is an adeno-associated virus (AAV) vector; or wherein at least one of the first vector and the second vector is AAV2.

62. (canceled)

63. The self-inactivating CRISPR-Cas system of claim 36, wherein the site-directed Cas9 polypeptide is Staphylococcus aureus Cas9 (SaCas9) or a variant thereof, or wherein the site-directed Cas9 polypeptide comprises the amino acid sequence set forth in SEQ ID NO: 1, SEQ ID NO: 2, SEQ ID NO: 3 or SEQ ID NO: 4.

64. (canceled)

65. The self-inactivating CRISPR-Cas system of claim 36, wherein the nucleotide sequence encoding the Cas9 polypeptide or variant thereof is codon optimized, or wherein the nucleotide sequence comprises SEQ ID NO: 79.

66. (canceled)

67. A genetically modified cell comprising the self-inactivating CRISPR-Cas system of claim 1.

68.-70. (canceled)

71. A method of genetically modifying a cell comprising the step of contacting the cell with the self-inactivating CRISPR-Cas system of claim 1.

72. (canceled)

73. A pharmaceutical composition comprising the self-inactivating CRISPR-Cas system of claim 1.

74. (canceled)

75. A nucleic acid for use in a self-inactivating CRISPR-Cas system comprising a codon optimized nucleotide sequence encoding a site-directed Cas9 polypeptide or variant thereof, wherein the codon optimized sequence comprises a self-inactivating (SIN) site and an adjacent Protospacer Adjacent Motif (PAM) within the open reading frame (ORF).

76. (canceled)

77. A nucleic acid for use in a self-inactivating CRISPR-Cas system comprising (i) a nucleotide sequence that encodes a site-directed Cas9 polypeptide or variant thereof, and (ii) a chimeric intron comprising a self-inactivating (SIN) site.

78.-79. (canceled)
